(19) United States
(12) Patent Application Publication
(10) Pub. No.: US 2019/0022221 A1
(43) Pub. Date: Jan. 24, 2019

(54) NON-INVASIVE SYSTEMS AND METHODS FOR TREATMENT OF A HOST CARRYING A VIRUS WITH PHOTOACTIVATABLE DRUGS

(71) Applicant: IMMUNOLIGHT, LLC., Detroit, MI (US)

(72) Inventors: Frederic Avery BOURKE, Aspen, CO (US); Harold WALDER, Oak Island, NC (US)

(73) Assignee: IMMUNOLIGHT, LLC., Detroit, MI (US)

(21) Appl. No.: 15/543,297

(22) PCT Filed: Jan. 13, 2016
(86) PCT No.: PCT/US2016/013208

§ 371 Date: Jul. 13, 2017

Related U.S. Application Data
(60) Provisional application No. 62/103,409, filed on Jan. 14, 2015.
(60) Provisional application No. 62/132,270, filed on Mar. 12, 2015.
(60) Provisional application No. 62/147,390, filed on Apr. 14, 2015.
(60) Provisional application No. 62/243,465, filed on Oct. 19, 2015.

(51) Int. Cl. A61K 41/00

(52) U.S. Cl. CPC A61K 41/0066; A61K 41/0019

(57) ABSTRACT

Products, compositions, systems, and methods for treating a subject carrying a virus or bacterium. The methods may be performed in situ in a non-invasive manner by application of an initiation energy to the subject thus producing an effect on or change to a target structure of the subject directly or indirectly. The methods may further be performed by application of an initiation energy to a subject to activate within the subject a photoactivatable drug directly or indirectly, optionally in the presence of one or more plasmonics active agents, thus treating the virus or bacterium. Also provided are kits containing products or compositions formulated or configured and systems for use in practicing these methods.

### CROSS REFERENCE TO RELATED APPLICATIONS

The present application is related to and claims priority to U.S. Ser. No. 62/103,409 filed Jan. 14, 2015 entitled “NON-INVASIVE SYSTEMS AND METHODS FOR TREATMENT OF A HOST CARRYING A VIRUS WITH PHOTOACTIVATABLE DRUGS,” the entire contents of which are hereby incorporated by reference. The present application is related to and claims priority to U.S. Ser. No. 62/132,270, filed Mar. 12, 2015, entitled “TUMOR IMAGING WITH X-RAYS AND OTHER HIGH ENERGY SOURCES USING AS CONTRAST AGENTS PHOTON-EMITTING PHOSPHORS HAVING THERAPEUTIC PROPERTIES”, the entire contents of which are hereby incorporated by references. This application is related to and claims priority to U.S. Ser. No. 62/147,390, filed Apr. 14, 2015, entitled “TUMOR IMAGING WITH X-RAYS AND OTHER HIGH ENERGY SOURCES USING AS CONTRAST AGENTS PHOTON-EMITTING PHOSPHORS HAVING THERAPEUTIC PROPERTIES”, the entire contents of which are hereby incorporated by reference. The present application is related to and claims priority to U.S. Ser. No. 62/243,465 filed Oct. 19, 2015, entitled “X-RAY PSORALEN ACTIVATED CANCER THERAPY (X-PACT),” the entire contents of which are hereby incorporated herein by reference.

The present application is related to U.S. Ser. No. 12/417,779 filed, Apr. 3, 2009, entitled “NON-INVASIVE SYSTEMS AND METHODS FOR IN-SITU PHOTOBIOMODULATION,” the entire contents of which are hereby incorporated by reference. The present application is related to U.S. Provisional application Ser. No. 61/955,131, filed May 18, 2014, the entire contents of which are hereby incorporated by reference. The present application is related to U.S. Provisional application Ser. No. 61/331,990, filed May 6, 2010, and U.S. Provisional application Ser. No. 61/443,019, filed Feb. 15, 2011, the entire contents of each of which are hereby incorporated by reference. The present application is also related to U.S. provisional patent application 61/161,328, filed Mar. 18, 2009; U.S. provisional patent application 61/259,940, filed Nov. 10, 2009; U.S. Provisional Application Ser. No. 60/954,263, filed Aug. 6, 2007, and 61/030,437, filed Feb. 21, 2008; U.S. application Ser. No. 12/059,484, filed Mar. 31, 2008; U.S. application Ser. No. 11/935,655, filed Nov. 6, 2007; U.S. Provisional Application Ser. No. 61/042,561, filed Apr. 4, 2008; 61/035,559, filed Mar. 11, 2008; and 61/080,140, filed Jul. 11, 2008; U.S. patent application Ser. No. 12/401,478 filed Mar. 10, 2009; U.S. patent application Ser. No. 11/935,655, filed Nov. 6, 2007; U.S. patent application Ser. No. 12/059,484, filed Mar. 31, 2008; U.S. patent application Ser. No. 12/389,946, filed Feb. 20, 2009; and U.S. patent application Ser. No. 12/417,779, filed Apr. 3, 2009, the entire contents of each of which is hereby incorporated by reference. This application is related to U.S. patent application Ser. No. 12/763,404 filed Apr. 20, 2010, the entire contents of which are hereby incorporated by reference. This application is related to U.S. patent application Ser. No. 12/843,188 filed Jul. 26, 2010, the entire contents of which are hereby incorporated by reference. This application is related to U.S. patent application Ser. No. 12/891,466 filed Sep. 27, 2010, the entire contents of which are hereby incorporated by reference. This application is related to U.S. patent application Ser. No. 12/943,787 filed Nov. 10, 2010, the entire contents of which are hereby incorporated by reference. This application is related to U.S. patent application Ser. No. 13/054,279 filed Jul. 13, 2011, the entire contents of which are hereby incorporated by reference. This application is related to U.S. patent application 61/505,849 filed Jul. 8, 2011, the entire contents of which are hereby incorporated by reference. This application is related to U.S. patent application Ser. No. 13/102,277 filed May 6, 2011, the entire contents of which are hereby incorporated by reference. This application is related to U.S. patent application Ser. No. 13/204,355 filed Aug. 5, 2011, the entire contents of which are hereby incorporated by reference. This application is related to U.S. patent application 61/735,754 filed Dec. 11, 2012, the entire contents of which are hereby incorporated by reference. This application is related to U.S. patent application 62/014,561 filed Jun. 19, 2014, the entire contents of which are hereby incorporated by reference. This application is related to U.S. patent application 61/792,125 filed Mar. 15, 2013, the entire contents of which are hereby incorporated by reference. This application is related to U.S. patent application 61/930,717 filed Jan. 23, 2014, the entire contents of which are hereby incorporated by reference. This application is related to U.S. patent application 61/955,131 filed Mar. 18, 2014, the entire contents of which are hereby incorporated by reference. This application is related to U.S. patent application 61/955,547 filed Mar. 19, 2014, the entire contents of which are hereby incorporated by reference. This application is related to U.S. patent application Ser. No. 14/103,084 filed Dec. 11, 2013, the entire contents of which are hereby incorporated by reference. This application is related to U.S. patent application Ser. No. 14/131,564 filed Jul. 11, 2014, the entire contents of which are hereby incorporated by reference. This application is related to U.S. patent application Ser. No. 14/206,337 filed Mar. 12, 2014, the entire contents of which are hereby incorporated by reference. This application is related to U.S. patent application 62/018,915 filed Jun. 30, 2014, the entire contents of which are hereby incorporated by reference. This application is related to U.S. patent application 62/038,674 filed Aug. 18, 2014, the entire contents of which are hereby incorporated by reference. This application is related to U.S. patent application 62/096,773 filed Dec. 24, 2014, the entire contents of which are hereby incorporated by reference. This application is related to national stage PCT/US2015/027058 filed Apr. 22, 2015, entitled “TUMOR IMAGING WITH X-RAYS AND OTHER HIGH ENERGY SOURCES USING AS CONTRAST AGENTS PHOTON-EMITTING PHOSPHORUS HAVING THERAPEUTIC PROPERTIES,” the entire contents of which are hereby incorporated herein by reference.

### BACKGROUND OF THE INVENTION

### Field of Invention

This invention relates to methods and systems for treating a disorder or condition in a subject.

### Discussion of the Background

Photobiomodulation also known as low level laser therapy (LLLT), cold laser therapy, and laser biostimulation, is an emerging medical and veterinary technique in which exposure to low-level laser light can stimulate or inhibit cellular function leading to beneficial clinical effects. The “best” combination of wavelength, intensity, duration and treatment interval is complex and sometimes controversial with different diseases, injuries and dysfunctions needing different treatment parameters and techniques.

Certain wavelengths of light at certain intensities (delivered by laser, light emitting diode LED or another monochromatic source) will, for example, aid tissue regeneration, resolve inflammation, relieve pain and boost the immune system. The exact mechanism is still being explored and debated but it is agreed that the mechanism is photochemical rather than heat-related. Observed biological and physiological effects include changes in cell membrane permeability, and up-regulation and down-regulation of adenosine triphosphate and nitric oxide.

Light-induced biological effects depend on the parameters of the irradiation (wavelength, dose, intensity, irradiation time, depth of a target cell, and continuous wave or pulsed mode, pulse parameters). (See, e.g., Karu I T, Low-Power Laser Therapy”, in Biomedical Photonics Handbook, Vo-Dinh T. Ed., CRC Press, Boca Raton, Fla., pp. 48-1 to 48-25, (2003)). Laser average power is typically in the range of 1-500 mW; some high peak power, short pulse width devices are in the range of 1-100 W with typically 200 ns pulse widths. The average beam irradiance then is typically 10 mW/cm²-5 W/cm². The wavelength is typically in the range 600-1000 nm. The red-to-near infrared (NIR) region is preferred for photobiomodulation. Other wavelengths may be also used, e.g., UV light for neurons and green light for prostate tissue. Maximum biological responses often occur when irradiated at 620, 680, 760, and 820-830 nm (Karu T I, et al., (1998). The Science of Low Power Laser Therapy. Gordon and Breach Sci. Publ., London). Large volumes and relatively deeper layers of tissues can be successfully irradiated by laser only (e.g., inner and middle ear diseases, injured sciatic or optical nerves, inflammations). The LEDs are used for irradiation of surface injuries.

The laser systems currently used for biostimulation do not allow performing photobiomodulation in a region deep within thick tissue without a surgical invasion. Laser therapy is mostly conducted in surface or near surface target cells and tissue because penetration of UV and red-to-N IR radiation used for photobiomodulation and photobiostimulation is no more than a few centimeters beneath the surface of the skin. In addition, imaging and stimulation of brain cells is mainly possible in thin brain slices, or a thin monolayer or suspension of cells. For deeper tissue laser therapy in situ, a subject undergoes various invasive surgical procedures, e.g., invasive insertion of a fiber via incisions into a fat layer or veins, implanting a radiation source in deep tissue, or implanting a glass window above the barrel cortex (Huber D., et al., Nature, 451:61-66 (2007)). It is further well recognized that another problem associated with the existing methods of photobiomodulation is in differentiation of normal cells from target cells.

Photodynamic therapy (PDT) is a treatment modality that uses a photosensitizing agent and laser light to kill cells. PDT is a relatively new light-based treatment, which has recently been approved by the United States Food & Drug Administration (FDA) for the treatment of both early and late-stage lung cancer. Other countries have approved PDT for treatment of various cancers as well. Unlike chemotherapy, radiation, and surgery, PDT is useful in treating all cell types, whether small cell or non-small cell carcinoma. PDT involves treatment of diseases such as cancer using light action on a special photoactive class of drugs, by photodynamic action in vivo to destroy or modify tissue [Dougherty T. J. and Levy J. G., “Photodynamic Therapy and Clinical Applications”, in Biomedical Photonics Handbook. Vo-Dinh T., Ed., CRC Press, Boca Raton Fla. (2003)]. PDT, which was originally developed for treatment of various cancers, has now been used to include treatment of pre-cancerous conditions, e.g. actinic keratoses, high-grade dysplasia in Barrett's esophagus, and non-cancerous conditions, e.g. various eye diseases, e.g. age related macular degeneration (AMD). Photodynamic therapy (PDT) is approved for commercialization worldwide both for various cancers (lung, esophagus) and for AMD.

The PDT process requires three elements: (1) a PA drug (i.e., photosensitizer), (2) light that can excite the photosensitizer and (3) endogenous oxygen. The putative cytotoxic agent is singlet oxygen, an electronically excited state of ground state triplet oxygen formed according to the Type II photochemical process, as follows.

### PA+hv→¹PA* (S) Excitation

¹PA* (S)→³PA* (T) Intersystem crossing for singlet to triplet state
³PA* (T)+O₂→¹O*₂+PA Energy transfer from the drug to singlet oxygen
where PA=photo-active drug at the ground state; ¹PA*(S)=excited singlet state; ³PA*(T)=excited triplet state; ¹O*₂=singlet excited state of oxygen

Because the triplet state has a relatively long lifetime (μsec to seconds) only photosensitizers that undergo efficient intersystem crossing to the excited triplet state will have sufficient time for collision with oxygen in order to produce singlet oxygen. The energy difference between ground state and singlet oxygen is 94.2 kJ/mol and corresponds to a transition in the near-infrared at ˜1270 nm. Most PA photosensitizers in clinical use have triplet quantum yields in the range of 40-60% with the singlet oxygen yield being slightly lower. Competing processes include loss of energy by deactivation to ground state by fluorescence or internal conversion (loss of energy to the environment or surrounding medium).

An important mechanism associated with PDT drug activity involves apoptosis in cells. Upon absorption of light, the photosensitiser (PS) initiates chemical reactions that lead to the direct or indirect production of cytotoxic species such as radicals and singlet oxygen. The reaction of the cytotoxic species with subcellular organelles and macromolecules (proteins, DNA, etc) lead to apoptosis and/or necrosis of the cells hosting the PDT drug. The preferential accumulation of PDT drug molecules in cancer cells combined with the localized delivery of light to the tumor, results in the selective destruction of the cancerous lesion. Compared to other traditional anticancer therapies, PDT does not involve generalized destruction of healthy cells. In addition to direct cell killing, PDT can also act on the vasculature, reducing blood flow to the tumor causing its necrosis. In particular cases it can be used as a less invasive alternative to surgery.

When laser light is administered via external illumination of tissue surfaces, the treatment effect of PDT is confined to a few millimeters (i.e. superficial). The reason for this superficial limitation is mainly the limited penetration of the visible light used to activate the photosensitizer. Thus, PDT is used to treat the surfaces of critical organs, such as lungs or intra-abdominal organs, without damage to the underlying structures. However, even these treatments require significantly invasive techniques to treat the surface of the affected organs. Clinical situations use the procedure in conjunction with surgical debulking to destroy remnants of microscopic or minimal gross disease. It is possible that the laser light and small amount of remaining microscopic and minimal gross disease results in too little or highly damaged structures.

Photopheresis has been successfully used for treatment of cell proliferation disorders. Exemplary cell proliferation disorders may include, but are not limited to, cancer, bacterial infection, immune rejection response of organ transplant, solid tumors, viral infection, autoimmune disorders (such as arthritis, lupus, inflammatory bowel disease, Sjogrens syndrome, multiple sclerosis) or a combination thereof, as well as aplastic conditions wherein cell proliferation is low relative to healthy cells, such as aplastic anemia. Of these, cancer is perhaps the most well known.

Extracorporeal photopheresis (ECP) is a leukapheresis-based immunomodulatory therapy that has been approved by the US Food and Drug Administration for the treatment of cutaneous T-cell lymphoma (CTCL). ECP, also known as extracorporeal photochemotherapy, is performed at more than 150 centers worldwide for multiple indications. Long-term follow-up data are available from many investigators that indicate ECP produces disease remission and improved survival for CTCL patients. In addition to CTCL, ECP has been shown to have efficacy in the treatment of other T-cell mediated disorders, including chronic graft versus host disease (GVHD) and solid organ transplant rejection. ECP use for the treatment of autoimmune disease, such as systemic sclerosis and rheumatoid arthritis, is also being explored.

ECP is generally performed using the UVAR XTS Photopheresis System developed by Therakos, Inc (Exton, Pa.). The process is performed through one intravenous access port and has 3 basic stages: (1) leukapheresis, (2) photoactivation, and (3) reinfusion, and takes 3-4 hours to complete. A typical treatment session would resemble the following sequence of events:

(1) One 16-gauge peripheral intravenous line or central venous access is established in the patient,

(2) Blood (225 mL) is passed through 3 cycles of leukapheresis, or 125 mL of blood is passed through 6 cycles, depending on the patient's hematocrit value and body size. At the end of each leukapheresis cycle, the red blood cells and plasma are returned to the patient;

(3) The collected WBCs (including approximately 5% of the peripheral blood mononuclear cells) are mixed with heparin, saline, and 8-methoxypsoralen (8-MOP), which intercalates into the DNA of the lymphocytes upon exposure to UVA light and makes them more susceptible to apoptosis when exposed to UVA radiation;

(4) The mixture is passed as a 1-mm film through a sterile cassette surrounded by UVA bulbs, resulting in an average UVA exposure of 2 J/cm²; and

(5) The treated WBC mixture is returned to the patient.

Over the past 20 years, on-going research has explored the mechanism of action of ECP. The combination of 8-MOP and UVA radiation causes apoptosis of the treated T cells and may cause preferential apoptosis of activated or abnormal T cells, thus targeting the pathogenic cells of CTCL or GVHD. However, given that only a small percentage of the body's lymphocytes are treated, this seems unlikely to be the only mechanism of action.

Other evidence suggests that ECP also induces monocytes to differentiate into dendritic cells capable of phagocytosing and processing the apoptotic T-cell antigens. When these activated dendritic cells are reinfused into the systemic circulation, they may cause a systemic cytotoxic CD8⁺ T-lymphocyte-mediated immune response to the processed apoptotic T-cell antigens.

Finally, animal studies indicate that photopheresis may induce antigen-specific regulatory T cells, which may lead to suppression of allograft rejection or GVHD.

Alternatively, a patient can be treated in vivo with a photosensitive agent followed by the withdrawal of a sample from the patient, treatment with UV radiation in vitro (ex vivo), and re-injecting the patient with the treated sample. This method is known for producing an autovaccine. A method of treating a patient with a photosensitive agent, exposing the patient to an energy source and generating an autovaccine effect wherein all steps are conducted in vivo has not been described. See WO 03/049801, U.S. Pat. No. 6,569,467; U.S. Pat. No. 6,204,058; U.S. Pat. No. 5,980,954; U.S. Pat. No. 6,669,965; U.S. Pat. No. 4,838,852; U.S. Pat. No. 7,045,124, and U.S. Pat. No. 6,849,058. Moreover, the side effects of extracorporeal photopheresis are well known and include nausea, vomiting, cutaneous erythema, hypersensitivity to sunlight, and secondary hematologic malignancy.

Researchers are attempting to use photopheresis in experimental treatments for patients with cardiac, pulmonary and renal allograft rejection; autoimmune diseases, and ulcerative colitis.

U.S. Pat. No. 5,829,448 describes sequential and simultaneous two photon excitation of photo-agents using irradiation with low energy photons such as infrared or near infrared light (NRI). A single photon and simultaneous two photon excitation is compared for psoralen derivatives, wherein cells are treated with the photo agent and are irradiated with NRI or UV radiation. The patent suggests that treating with a low energy irradiation is advantageous because it is absorbed and scattered to a lesser extent than UV radiation.

Chen et al., J. Nanosci. and Nanotech., 6:1159-1166 (2006); Kim et al., JACS, 129:2669-2675 (2007); U.S. 2002/0127224; and U.S. Pat. No. 4,979,935 each describe methods for treatment using various types of energy activation of agents within a subject. However, each suffers from the drawback that the treatment is dependent on the production of singlet oxygen to produce the desired effect on the tissue being treated, and is thus largely indiscriminate in affecting both healthy cells and the diseased tissue desired to be treated.

U.S. Pat. No. 6,908,591 describes methods for sterilizing tissue with irradiation to reduce the level of one or more active biological contaminants or pathogens, such as viruses, bacteria, yeasts, molds, fungi, spores, prions or similar agents responsible, alone or in combination, for transmissible spongiform encephalopathies and/or single or multicellular parasites, such that the tissue may subsequently be used in transplantation to replace diseased and/or otherwise defective tissue in an animal. The method may include the use of a sensitizer such as psoralen, a psoralen-derivative or other photosensitizer in order to improve the effectiveness of the irradiation or to reduce the exposure necessary to sterilize the tissue.

U.S. Pat. No. 5,957,960 describes a two-photon excitation device for administering a photodynamic therapy to a treatment site within a patient's body using light having an infrared or near infrared waveband.

U.S. Pat. No. 6,235,508 describes antiviral applications for psoralens and other photoactivatable molecules. It teaches a method for inactivating viral and bacterial contaminants from a biological solution. The method includes mixing blood with a photosensitizer and a blocking agent and irradiating the mixture to stimulate the photosensitizer, inactivation of substantially all of the contaminants in the blood, without destroying the red blood cells. The blocking agent prevents or reduces deleterious side reactions of the photosensitizer, which would occur if not in the presence of the blocking agent. The mode of action of the blocking agent is not predominantly in the quenching of any reactive oxygen species, according to the reference.

U.S. Pat. No. 6,235,508 suggests that halogenated photosensitizers and blocking agents might be suitable for replacing 8-methoxypsoralen (8-MOP) in photopheresis and in treatment of certain proliferative cancers, especially solid localized tumors accessible via a fiber optic light device or superficial skin cancers. However, the reference fails to address any specific molecules for use in treating lymphomas or any other cancer. Instead, the reference suggests a process of photopheresis for antiviral treatments of raw blood and plasma.

U.S. published application 2002/0127224 describes a method for a photodynamic therapy comprising administering light-emitting nanoparticles and a photoactivatable agent, which may be activated by the light re-emitted from the nanoparticles via a two-photon activation event. An initiation energy source is usually a light emitting diode, laser, incandescent lamp, or halogen light, which emits light having a wavelength ranging from 350 to 1100 nm. The initiation energy is absorbed by the nanoparticles. The nanoparticles, in turn, re-emit light having a wavelength from 500 to 1100 nm, preferably, UV-A light, wherein the re-emitted energy activates the photoactivatable agent. Kim et al., (JACS, 129:2669-75, Feb. 9, 2007) describes indirect excitation of a photosensitizing unit (energy acceptor) through fluorescence resonance energy transfer (FRET) from the two-photon absorbing dye unit (energy donor) within an energy range corresponding to 300-850 nm.

### Psoralens and Related Compounds

U.S. Pat. No. 6,235,508 describes that psoralens are naturally occurring compounds which have been used therapeutically for millennia in Asia and Africa. The action of psoralens and light has been used to treat vitiligo and psoriasis (PUVA therapy; Psoralen Ultra Violet A). Psoralen is capable of binding to nucleic acid double helices by intercalation between base pairs; adenine, guanine, cytosine and thymine (DNA) or uracil (RNA). Upon sequential absorption of two UV-A photons, psoralen in its excited state reacts with a thymine or uracil double bond and covalently attaches to both strands of a nucleic acid helix. The crosslinking reaction appears to be specific for a thymine (DNA) or a uracil (RNA) base. Binding may proceed when psoralen is intercalated in a site containing thymine or uracil, but an initial photoadduct must absorb a second UVA photon to react with a second thymine or uracil on the opposing strand of the double helix in order to crosslink each of the two strands of the double helix, as shown below. This is a sequential absorption of two single photons as shown, as opposed to simultaneous absorption of two or more photons.

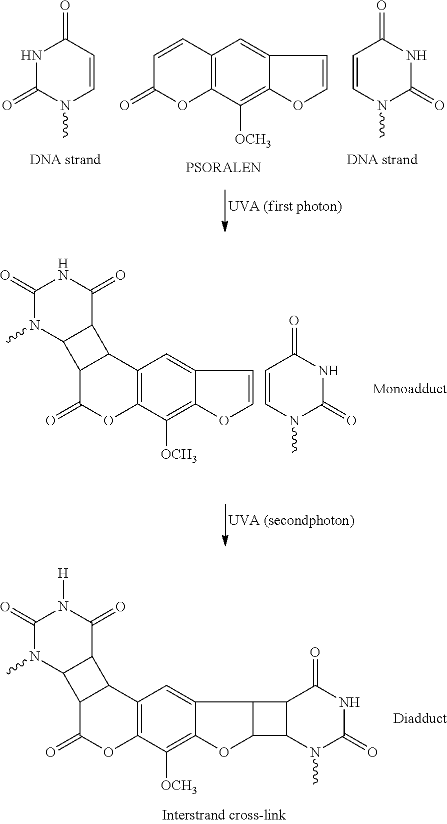

U.S. Pat. No. 4,748,120 of Wiesehan is an example of the use of certain substituted psoralens by a photochemical decontamination process for the treatment of blood or blood products.

Additives, such as antioxidants are sometimes used with psoralens, such as 8-MOP, AMT and I-IMT, to scavenge singlet oxygen and other highly reactive oxygen species formed during photoactivation of the psoralens. It is well known that UV activation creates such reactive oxygen species, which are capable of seriously damaging otherwise healthy cells. Much of the viral deactivation may be the result of these reactive oxygen species rather than any effect of photoactivation of psoralens.

Some of the best known photoactivatable compounds are derivatives of psoralen or coumarin, which are nucleic acid intercalators. For psoralens and coumarins, this chemical pathway is likely to lead to the formation of a variety of ring-opened species, such as shown below for coumarin:

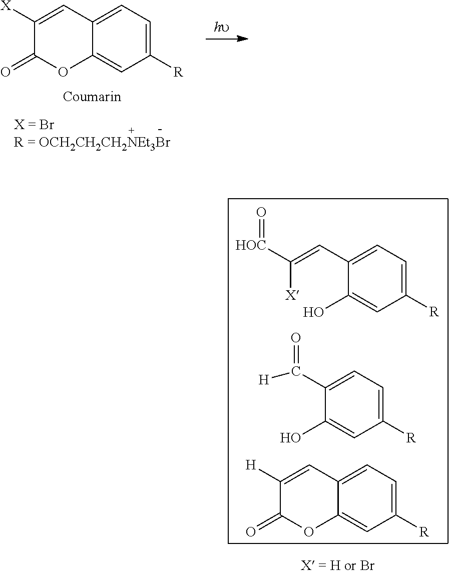

Midden (W. R. Midden, Psoralen DNA photobiology, Vol II (ed. F. P. Gaspalloco) CRC press, pp. 1. (1988) has presented evidence that psoralens photoreact with unsaturated lipids and photoreact with molecular oxygen to produce active oxygen species such as superoxide and singlet oxygen that cause lethal damage to membranes.

U.S. Pat. No. 6,235,508 describes that 8-MOP and AMT are unacceptable photosensitizers, because each indiscriminately damages both cells and viruses. Studies of the effects of cationic side chains on furocoumarins as photosensitizers are reviewed in Psoralen DNA Photobiology, Vol. I, ed. F. Gaspano, CRC Press, Inc., Boca Raton, Fla., Chapter 2. U.S. Pat. No. 6,235,508 gleans the following from this review: most of the amino compounds had a much lower ability to both bind and form crosslinks to DNA compared to 8-MOP, suggesting that the primary amino functionality is the preferred ionic species for both photobinding and crosslinking.

U.S. Pat. No. 5,216,176 describes a large number of psoralens and coumarins that have some effectiveness as photoactivated inhibitors of epidermal growth factor. Halogens and amines are included among the vast functionalities that could be included in the psoralen/coumarin backbone. This reference is incorporated herein by reference in its entirety.

U.S. Pat. No. 5,984,887 describes using extracorporeal photopheresis with 8-MOP to treat blood infected with CMV. The treated cells as well as killed and/or attenuated virus, peptides, native subunits of the virus itself (which are released upon cell break-up and/or shed into the blood) and/or pathogenic noninfectious viruses are then used to generate an immune response against the virus, which was not present prior to the treatment.

### Other Photoactive Compounds

Other photoactive or photoactivatable compounds are known in the art. Of these, an article by Warfield et al entitled “Ebola Virus Inactivation with Preservation of Antigenic and Structural Integrity by a Photoinduable Alkylating Agent,” J. Infect. Dis. 2007 Nov. 15; 196 Suppl 2:S276-83 describes the treatment of the Zaire Ebola virus (ZEBOV) ex situ by extraction of infected blood from a mouse and exposure of the extracted blood to UV light (310 to 360 nm) with the blood containing an alkylating agent, in this case iodonophthylazide (INA) to inactivate the ZEBOV. Mice treated with the inactivated Ebola virus were resistant to exposure to the Ebola virus. These authors reported that INA is hydrophobic compound that preferentially partitions into lipid bilayers of the Ebola virus. These authors reported that the “INA treatment renders ZEBOV completely noninfectious without structural perturbation” and that “INA-inactivated ZEBOV was immunogenic and protected mice from lethal challenge.”

U.S. Pat. No. 7,049,110 entitled “Inactivation of West Nile virus and malaria using photosensitizers” describes the inactivation of microorganisms in fluids or on surfaces, preferably the fluids that contain blood or blood products and biologically active proteins. An effective, non-toxic amount of a photosensitizer was added to the fluid, and the fluid was exposed to photoradiation sufficient to activate the photosensitizer whereby microorganisms were inactivated.

The '110 patent describes a 7,8-dimethyl-10-ribityl isoalloxazine photosensitizers and other photosensitizers including endogenous alloxazine or isoalloxazine photosensitizers. The '110 patent describes the treatment of a host carrying various microorganisms including viruses (both extracellular and intracellular), bacteria, bacteriophages, fungi, blood-transmitted parasites such as malaria, and protozoa. Exemplary viruses include acquired immunodeficiency (HIV) virus, hepatitis A, B and C viruses, sinbis virus, cytomegaloviris, vesicular stomatitis virus, herpes simplex viruses, e.g. types I and II, human T-lymphotropic retroviruses, HTLV-III, lymphadenopathy virus LAV/IDAV, parvovirus, transfusion-transmitted (TT) virus, Epstein-Barr virus, West Nile virus and others known to the art. Bacteriophages include ΦX174, Φ6, λ, R17, T4, and T2. Exemplary bacteria include P. aeruginosa, S. aureus, S. epidermis, L. monocytogenes, E. coli, K. pneumonia and S. marcescens. One particular class of microorganisms is non-screened microorganisms—those microorganisms that are not screened by current blood banking processes. Some non-screened microorganisms include malaria and West Nile virus. One class of microorganisms include those transmitted by mosquitoes, including malaria and West Nile virus.

The '110 patent describes that the preferable use endogenous photosensitizers, including endogenous photosensitizers which function by interfering with nucleic acid replication. In 7,8-dimethyl-10-ribityl isoalloxazine, the chemistry believed to occur between 7,8-dimethyl-10-ribityl isoalloxazine and nucleic acids does not proceed via singlet oxygen-dependent processes (i.e. Type II mechanism), but rather by direct sensitizer-substrate interactions (Type I mechanisms). In addition, 7,8-dimethyl-10-ribityl isoalloxazine appears not to produce large quantities of singlet oxygen upon exposure to UV light, but rather exerts its effects through direct interactions with substrate (e.g., nucleic acids) through electron transfer reactions with excited state sensitizer species.

An article by Sharma et al. entitled “Safety and protective efficacy of INA-inactivated Venezuelan equine encephalitis virus: Implication in vaccine development,” in Vaccine, volume 29, issue 5, 29 Jan. 2011, pages 953-959, described that that hydrophobic alkylating compound, 1,5-iodonaphthyl-azide (INA) can efficiently inactivate the virulent strain of Venezuelan equine encephalitis virus (VEEV), upon exposure of the INA to “full light conditions.” Sharma et al. further demonstrated the protective efficacy of INA-inactivated V3000 and V3526 to not cause disease in suckling mice and to induce an anti-VEEV antibody response which protected mice from a virulent VEEV challenge. Sharma et al. reported that none of the mice which received INA-inactivated V3526 showed any clinical symptoms of disease such as, hunched posture, stunted growth, lethargic or paralysis and grew similar to that of the control mice.

An article by Heilman et al. entitled “Light-Triggered Eradication of Acinetobacter baumannii by Means of NO Delivery from a Porous Material with an Entrapped Metal Nitrosyl” in J. Am. Chem. Soc., 2012, 134 (28), pp 11573-11582 (May 11, 2012) describes photoactive manganese nitrosyl, namely [Mn(PaPy3)(NO)](ClO4) ({Mn—NO}), loaded into the columnar pores of an MCM-41 host. Heliman et al. report that, when suspensions of the loaded materials in saline solution were exposed to low-power (10-100 mW) visible light, rapid release of NO was observed. Reports on this work detail tests in which conditions in an infected wound were simulated such that bacteria (Acinetobacter baumannii) grew throughout a 1.1-millimeter-thick layer of soft agar. To which aluminosilicate powder, with and without the photoactive manganese nitrosyl compound, was applied. The released nitric oxide effectively cleared the bacteria from the treated areas of the plates, showing that the nitric oxide easily penetrated through the agar layer. The amount of light used to activate the compound was 100 milliWatts per square centimeter.

U.S. Pat. No. 8,268,602 entitled “CELLULAR AND VIRAL INACTIVATION” describes procedures for providing compositions of inactivated viruses, bacteria, fungi, parasites and tumor cells that can be used as vaccines, as well as methods for making such inactivated viruses, bacteria, fungi, parasites and tumor cells are also provided. More specifically, the '602 patent describes methods for inactivating an infective agent or cancer cell that involve exposing the agent or cell to a hydrophobic photoactivatable compound, for example, 1,5-iodonaphthylazide (INA) activated by ultraviolet light.

The above-noted patents, patent applications, and articles are incorporated by reference in their entirety herein. All of the following patents, patent applications, and articles noted herein are also incorporated by reference in their entirety herein.

### SUMMARY OF THE INVENTION

In one embodiment of the invention, there is provided a method for treating a subject carrying a virus or a bacterium. The method provides within the subject 1) one or more light emitters capable of emitting at least one wavelength of light and 2) at least one photoactive or photoactivatable drug for treatment of the subject carrying the virus or the bacterium. The method applies initiation energy from at least one source to a target inside the subject to activate the light emitters. From the at least one wavelength of light, the method activates inside the subject the at least one photoactive or photoactivatable drug. Inside the subject, the method reacts the activated drug with the virus or the bacterium to inactivate the virus or the bacterium to thereby treat the subject.

In one embodiment of the invention, there is provided a method for treating a subject carrying a virus or a bacterium. The method provides within the subject at least one photoactive or photoactivatable drug for treatment of a subject carrying the virus and applies initiation energy from at least one source to a target inside the subject. The method activates directly or indirectly the at least one photoactive or photoactivatable drug at the target inside the subject. Inside the subject, the activated drug reacts with the virus or bacterium to inactivate the virus or bacterium to thereby treat the subject carrying the virus.

In one embodiment, there is provided a method for treating a subject carrying a virus or a bacterium. The method provides within a respiratory track of the subject at least one photoactive or photoactivatable drug for treatment the subject carrying the virus or the bacterium, and applies initiation energy from at least one source to the respiratory track. The method activates directly or indirectly the at least one photoactive or photoactivatable drug at the target inside the respiratory track. Inside the respiratory track, the activated drug can reacts with the virus or bacterium to inactivate the virus or the bacterium to thereby treat the subject.

In one embodiment, there is provided a method for treating a subject carrying a virus or a bacterium. The method provides within lymph nodes of the subject at least one photoactive or photoactivatable drug for treatment the subject carrying the virus or the bacterium, and applies initiation energy from at least one source to the lymph nodes. The at least one photoactive or photoactivatable drug is activated directly or indirectly at the target inside the lymph nodes. Inside the lymph nodes, the activated drug reacts with the virus or bacterium to inactivate the virus or the bacterium to thereby treat the subject.

It is to be understood that both the foregoing general description of the invention and the following detailed description are exemplary, but are not restrictive of the invention.

### BRIEF DESCRIPTION OF THE FIGURES

A more complete appreciation of the invention and many of the attendant advantages thereof will be readily obtained as the same becomes better understood by reference to the following detailed description when considered in connection with the accompanying drawings, wherein:

FIG. 1 provides an exemplary electromagnetic spectrum in meters (1 nm equals 10⁻⁹ meters).

FIG. 2A and FIG. 2B are graphical representations of the depth of penetration of various wavelengths of energy into living tissue.

FIG. 3 illustrates a system according to one exemplary embodiment of the invention.

FIG. 3-1 illustrates an exemplary system according to one embodiment of the invention for producing a photo-reactive change in a medium.

FIG. 4 illustrates an exemplary computer implemented system according to an embodiment of the invention.

FIG. 5 illustrates an exemplary computer system for implementing various embodiments of the invention.

### DETAILED DESCRIPTION OF THE INVENTION

The invention described below sets forth a novel method of modifying a target structure which mediates or is associated with a biological activity, which includes treating a condition, disorder or disease in a subject (preferably but not limited to viral infections). The subjects treatable by the invention include animals, birds, and humans.

All methods and materials similar or equivalent to those described herein can be used in the practice or testing of the invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the terms “at” or “about,” as used herein when referring to a measurable value or metric is meant to encompass variations of 20%, 10%, 5%, 1%, 0.5%, or even 0.1% of the specified amount, for example a specified ratio, a specified thickness, a specified phosphor size, etc. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Generally, one aspect of the invention includes a method for treating a subject carrying a virus. The method provides within the subject 1) one or more light emitters capable of emitting at least one wavelength of light and 2) at least one photoactive drug for treatment a subject carrying the virus. The method applies initiation energy from at least one source to a target inside the subject to activate the light emitters, and generates at the target inside the subject the at least one wavelength of light. From the at least one wavelength of light, the at least one photoactive drug is activated inside the subject to thereby treat the subject carrying the virus.

Alternatively, or in addition, another aspect of the invention includes a method for treating a subject carrying a virus in which the method provides within the subject at least one photoactive drug for treatment of the subject carrying the virus and applies initiation energy from at least one source to a target inside the subject. The at least one photoactive drug is activated directly or indirectly at the target inside the subject to thereby treat the subject carrying the virus.

Mechanisms included in the invention can involve photoactivation of a drug such as a psoralen or its derivatives or an alkylating agent. Mechanisms included in the invention can involve the formation of highly reactive oxygen species, such as singlet oxygen. Any of these mechanisms can be used in combination or selectively to treat a subject carrying viruses and associated disorders or symptoms thereof. In one embodiment, one wavelength can be used to activate an alkylating agent (e.g., iodonophthylazide) for its attachment to a virus, while another different wavelength can be used to activate a psoralen (or a derivative or substitute thereof) for treatment of a bacterial infection or other disorders in the patient. In one embodiment, one wavelength can be used to activate an alkylating agent or a psoralen, while another wavelength is used for a different purpose such as for example production of singlet oxygen (i.e., highly reactive oxygen species) or for production of sterilizing UV light or to promote cell growth or reduce inflammation, etc.

In various embodiments, one or more wavelengths could be used for treatment a host or arrest of viruses such as Ebola, West Nile, encephalitis, HIV, etc., and/or for the regulation and control of biological responses having varying degrees of apoptosis (the process of programmed cell death PCD) and necrosis (the premature death of cells and living tissue typically from external factors). In necrosis, factors external to the cell or tissue, such as infection, toxins, or trauma that result in the unregulated digestion of cell components. In contrast, apoptosis is a naturally occurring programmed and targeted cause of cellular death. While apoptosis often provides beneficial effects to the organism, necrosis is almost always detrimental and may be fatal.

Cells that die due to necrosis do not follow the apoptotic signal transduction pathway, but rather various receptors are activated that result in the loss of cell membrane integrity and an uncontrolled release of products of cell death into the intracellular space. This initiates in the surrounding tissue an inflammatory response which prevents nearby phagocytes from locating and eliminating the dead cells by phagocytosis. For this reason, it is often necessary to remove necrotic tissue surgically, a procedure known as debridement. Untreated necrosis results in a build-up of decomposing dead tissue and cell debris at or near the site of the cell death. A classic example is gangrene.

In various embodiments of this invention, the alkylating agent can be at least one or more of drugs from the iodonophthylazide family, such as 1,5-iodonaphthylazide (INA). These photoactivatable compounds are non-toxic, hydrophobic compounds that can penetrate into the innermost regions of biological membrane bilayers and selectively accumulate in such inner membrane regions.

Upon irradiation with light, generated inside or nearby the membrane region, it is believed that a reactive derivative of the compound is generated that binds to membrane proteins deep in the lipid bilayer. This process would (similar to that in the '602 patent) inactivate integral membrane proteins embedded in the membrane while maintaining the structural integrity and activity of the proteins that protrude from the extracellular surface of the membrane. In one aspect of the invention, the inactivated agent constitutes a vaccine created inside the subject animal or bird or human with the vaccine specific to the viral or bacterial infection of the animal or bird or human.

The Ebola virus is believed to fuse with cells lining the respiratory track, eyes, or body cavities. Once inside these cells, the virus's genetic contents are released into the cell, where it takes over the cell and replicates itself. New copies of the virus are released from the cell.

In various embodiments of this invention, a photoactive drug such as a psoralen or its derivatives is used separately or in conjunction with at least one alkylating agent. When using a psoralen, the psoralen is photactivated inside the cell by ultraviolet or visible light generated within the cell or nearby the cell. The activated psoralen attaches to the virus's genetic contents, prevents its replication, and causes local cell death (one form of treatment). Alternatively or in addition, the psoralen-inactivated virus can induce an autoimmune response from the animal or bird or human resulting in the body effectively eliminating untreated viruses in other regions of the body.

Accordingly, as provided herein, a photoactivatable hydrophobic compound useful in various embodiments of the invention can be one of the following formula (I) and can be used to inactivate viruses, parasites and tumor cells and other infectious structures and microorganisms when activated inside an animal or bird or human subject:

X—Ar—Y (I),

wherein:

Ar is a hydrophobic moiety; and

X and Y are each independently hydrogen or a reactive group, provided that at least one of X or Y is a reactive group.

The Ar hydrophobic moiety can be any moiety that preferentially partitions out of an aqueous environment and into a cellular or viral membrane. Examples of Ar hydrophobic moieties include linear, branched, cyclic and acyclic hydrocarbons and combinations thereof. The cyclic groups employed can be non-aromatic or aromatic ring moieties. For example, the Ar hydrophobic moiety can be a fatty acid, alkyl, adamantane, phenyl, naphthyl, anthracene, pyrene, phenanthracene or similar moiety.

The X and Y reactive groups can be functional groups that are chemically reactive (or that can be made or activated to be chemically reactive) with functional groups typically found in biological materials, or with functional groups that can be readily converted to chemically reactive groups using methods well known in the art. In one embodiment, the X and/or Y reactive groups are separately azido (—N₃), halo (Cl, Br or I), halo lower alkyl (e.g. CF₃), diazirene, azidocarbonyloxy (—O—CO—N₃), haloacetamide (—NH—(C═O)—CH₂—Z), where Z is Cl, Br or I. Alternatively, the reactive groups are separately amine, maleimide, isocyanato (—N═C═O), isothiocyanato (—N═C═S), acyl halide, succinimidyl ester, or sulfosuccinimidyl ester.

In another embodiment of the invention, the reactive groups can be carboxylic acid (COOH), or derivatives of a carboxylic acid. An appropriate derivative of a carboxylic acid can include an alkali or alkaline earth metal salt of carboxylic acid. Alternatively, the reactive groups can be reactive derivatives of a carboxylic acid (—COOR), where the reactive group R is one that activates the carbonyl group of —COOR toward nucleophilic displacement. In particular, R is any group that activates the carbonyl towards nucleophilic displacement without being incorporated into the final displacement product. Examples of COOR groups include esters of phenol or naphtol that are further substituted by at least one strong electron withdrawing group, or carboxylic acid activated by carbodiimide, or constitute acyl chloride, azido, succinimidyl or sulfosuccinimidyl ester. Additional charged groups include, among others, sulfonyl halides, sulfonyl azides, alcohols, thiols, semicarbazides, hydrazines or hydroxylamines.

Examples of photoactivatable hydrophobic compounds that can be used in the invention include (but are not limited to) the following compounds:

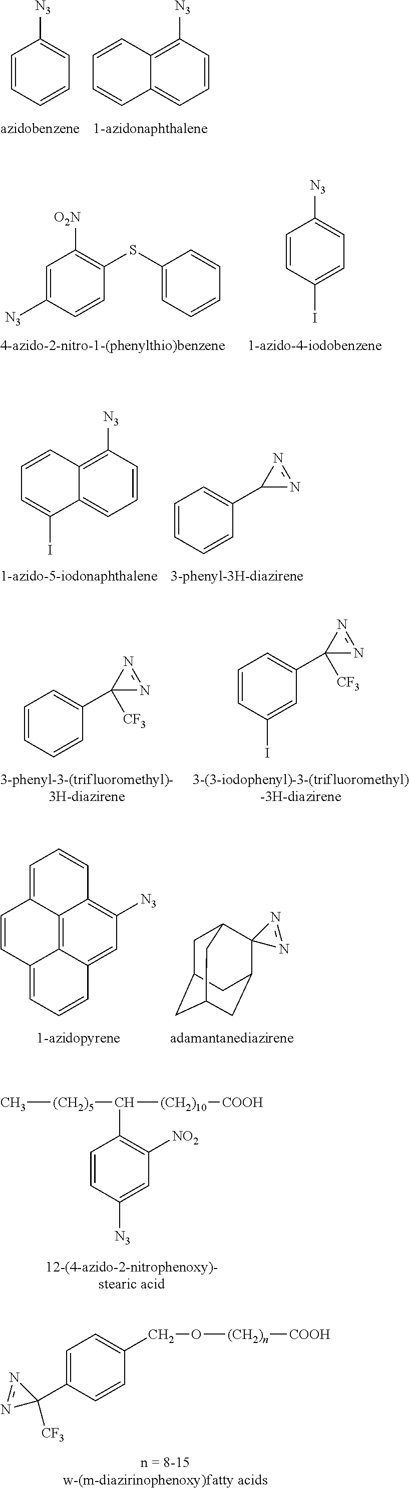

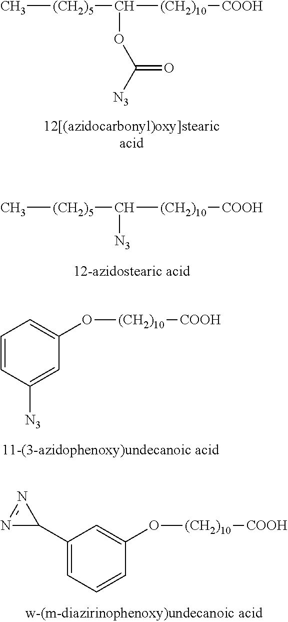

In one embodiment of the invention, 1,5-iodonaphthyl azide (INA) is employed as a photoactivatable hydrophobic compound. INA is a nontoxic hydrophobic compound. The structure for 1,5-iodonaphthyl azide (INA) is provided below.

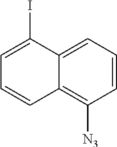 [SMILES: [N-]=[N+]=Nc1cccc2c(I)cccc12]

Upon exposure to cells, the photoactivatable hydrophobic compounds can penetrate into the innermost regions of biological membrane bilayers and will accumulate selectively in these regions. Upon irradiation with ultraviolet light (e.g., 320 to 400 nm) generated (or otherwise provided) internally within the animal or bird or human subject, it is believed that a reactive derivative is generated that binds to membrane proteins deep in the lipid bilayer. While the invention is not limited by the following, this process is believed to specifically inactivate integral membrane proteins embedded in the membrane while maintaining the integrity and activity of the proteins that protrude from the extracellular surface of the membrane.

In another embodiment of the invention, the photoactivatable hydrophobic compounds of the invention can be used for inactivation of viruses, bacteria, parasites and tumor cells using visible light. However, when visible light is used a photosensitizer, a chromophore is typically needed unless the photoactive drug is developed to be activated directly by visible light. A photosensitizer chromophore has an absorption maximum in the visible light range and can photosensitize the photoactivatable hydrophobic compounds of the invention. In general, the photosensitizer chromophores have absorption maxima in the range of about 450 to about 525 nm or about 600 to about 700 nm. Suitable photosensitizer chromophores can include one or more of a porphyrin, chlorin, bacteriochlorin, purpurin, phthalocyanine, naphthalocyanine, merocyanines, carbocyanine, texaphyrin, non-tetrapyrrole, or other photosensitizers known in the art. Specific examples of photosensitizer chromophores include fluorescein, eosin, bodipy, nitro-benzo-diazol (NBD), erythrosine, acridine orange, doxorubicin, rhodamine 123, picoerythrin and the like.

As provided in various embodiments of the invention, viruses, bacteria, parasites and tumor cells and other infectious structures and microorganisms can be inactivated by exposure to photoactivatable hydrophobic compounds which were themselves activated by light generated internally within the animal or bird or human subject. In various embodiments, the photoactivatable hydrophobic compound is 1,5-iodonaphthyl azide (INA) or a related compound. In one embodiment of the invention, the virus, parasite or tumor cell is contacted with the recently photoactivated hydrophobic compound, which was photoactivated by ultraviolet light generated internally using the energy modulation agents of the invention. If the virus, parasite, tumor cell or other infectious structures and microorganisms are contacted with both the photoactivatable hydrophobic compound and a photosensitizer chromophore that absorbs visible light, then visible light generated internally using the energy modulation agents of the invention can photoactivate the photoactivatable hydrophobic compound. Accordingly, in one embodiment, exposure to internally generated ultraviolet light directly photoactivates the photoactivatable hydrophobic compound within viral and cellular membranes. In one embodiment, exposure to internally generated visible light first photoactivates the photosensitizer chromophore, which then activates or photosensitizes the photoactivatable hydrophobic compound within viral or cellular membranes.

In either case, a reactive derivative of the photoactivatable hydrophobic compound is generated that binds to membrane proteins deep within the lipid bilayer. This process is believed to cause specific inactivation of integral membrane proteins embedded in the membrane, while maintaining the integrity and activity of proteins that protrude outside of the membrane.

In one embodiment of the invention, the ultraviolet light internally generated has a wavelength that is generally not absorbed by proteins and nucleic acids. Such a wavelength of ultraviolet light internally generated does not cause substantial damage to such proteins and nucleic acids. Thus, for example, the wavelength internally generated can be about 320 nm to about 400 nm. In some embodiments, the wavelength of light internally generated is about 330 nm to about 380 nm. In other embodiments, the wavelength internally generated is about 340 nm to about 360 nm.

Visible light of an appropriate wavelength can be used when a photosensitizer chromophore is employed that is incubated with or is localized in the vicinity of the hydrophobic photoactivatable compound. In general, the photosensitizer chromophores have absorption maxima in the range of about 450 to about 525 nm or about 600 to about 700 nm. Light internally generated for photoactivation of the photosensitizer chromophore or the hydrophobic derivative can be produced from the various energy modulation agents described herein.

While the invention is not so limited, INA is known to penetrate into the inner most segments of membrane bilayers and accumulate selectively in this domain. Upon irradiation of the organism or cell with ultraviolet light internally generated (e.g., 320-400 nm), INA is photoactivated in the membrane to generate a reactive derivative that binds to membrane proteins deep within the lipid bilayer. While the invention is not so limited, this process can cause specific inactivation of integral membrane proteins embedded in the membrane, while maintaining the integrity and activity of proteins that protrude outside the membrane.

The invention with internally generated light can provide a method that can inactivate a wide variety of viruses, bacteria, parasites and tumor cells in a way that the inactivated species can be safely used as immunological compositions or vaccines to inhibit the disease they cause. The inactivation kills the organism or cell in a specific manner that maintains its structure and conformation. Hence, the structure of the inactivated virus/cell is similar to that of the live virus/cell. In this way, the immunogenicity of the organism or cell as a whole is maintained and can be safely used to stimulate the immune system of a subject animal or bird or patient. Similarly, in one aspect of the invention, the inactivated viruses, bacteria, cancer cells, or parasites generated inside the animal or bird or human subject can be used for vaccination without causing disease or other negative side effects.

Hence, the INA internal treatment procedures generate inactive viruses inside the subject that can be used in a manner similar to aldrithiol inactivated HIV (developed by the AIDS vaccine program SAIC). Alternatively, the INA-internal-inactivation procedures of this invention can be used in conjunction with aldrithiol inactivation procedures to generate inactive HIV that comply with the requirements of the FDA. Thus, in one aspect of this invention, two mechanistically independent methods of inactivation can be used to provide a prophylactic AIDS or HIV vaccine.

In one aspect of the invention, prevention or treatment of microbial infections, viral infections, parasitic infections, prion infection or cancer is intended to include the alleviation of or diminishment of at least one symptom typically associated with the infection or cancer. Prevention or treatment also includes alleviation or diminishment of more than one symptom. Ideally, treatment with the internally inactivated agents of the invention generates immunity in the animal or bird or human towards the agent while prevention by the inactivated agents of the invention substantially eliminates the symptoms associated with the infection or cancer.

In various embodiments of the invention, infections that can be treated by the present internally inactivated agents include infections by any target infectious organisms and structures that can infect a mammal or other animal or a bird. Such target infectious organisms and structures include, but are not limited to, any virus, bacterium, fungus, single cell organism, prion conformations or parasite that can infect an animal, including mammals. For example, target microbial organisms include viruses, bacteria, fungi, yeast strains and other single cell organisms. In another embodiment, the inactivated agents of the invention can give rise to immunity against both gram-negative and gram-positive bacteria.

In one aspect of the invention, the internal treatment of, or prevention of, viral, bacterial, fungal, microbial, prion or parasitic infections is intended to include the alleviation of or diminishment of at least one symptom typically associated with the infection. The internal treatment also includes alleviation or diminishment of more than one symptom. The internal treatment may cure the infection, e.g., it may substantially prevent the infection and/or eliminate the symptoms associated with the infection.

Exemplary viral infections that can be treated by the internally inactivated agents of this invention include infections by any virus that can infect animals (including but not limited to mammals or birds), including enveloped and non-enveloped viruses, DNA and RNA viruses, viroids, and prions. Hence, for example, infections or unwanted levels of the following viruses and viral types can be treated internally: human immunodeficiency virus (HIV), simian immunodeficiency virus (SIV), hemorrhagic fever viruses, hepatitis A virus, hepatitis B virus, hepatitis C virus, poxviruses, herpes viruses, adenoviruses, papovaviruses, parvoviruses, reoviruses, orbiviruses, picomaviruses, rotaviruses, alphaviruses, rubiviruses, influenza virus type A and B, flaviviruses, coronaviruses, paramyxoviruses, morbilliviruses, pneumoviruses, rhabdoviruses, lyssaviruses, orthmyxoviruses, bunyaviruses, phleboviruses, nairoviruses, hepadnaviruses, arenaviruses, retroviruses, enteroviruses, rhinoviruses and the filovirus.

Infections or unwanted levels of the following target viruses and viral types that are believed to have potential as biological weapons can be treated, prevented or addressed by the internally inactivated agents of this invention: hemorrhagic fever viruses (HFVs), Chikungunya virus, Japanese encephalitis virus, Monkey pox virus, variola virus, Congo-Crimean hemorrhagic fever virus, Junin virus, Omsk hemorrhagic fever virus, Venezuelan equine encephalitis virus, Dengue fever virus, Lassa fever virus, Rift valley fever virus, Western equine encephalitis virus, Eastern equine encephalitis virus, Lymphocytic choriomeningitis virus, Russian Spring-Summer encephalitis virus, White pox, Ebola virus, Machupo virus, Smallpox virus, Yellow fever virus, Hantaan virus, Marburg virus, and Tick-borne encephalitis virus.

Similarly, infections or unwanted levels of the following examples of target microbial organisms can be treated by the internally inactivated agents of this invention: Aeromonas spp. (including, for example, Aeromonas hydrophila, Aeromonas caviae and Aeromonas sobria), Bacillus spp. (including, for example, Bacillus cereus, Bacillus anthracis and Bacillus thuringiensis), Bacteroides spp. (including, for example, B. fragilis, B. thetaiotaomicron, B. vulgatus, B. ovatus, B. distasonis, B. uniformis, B. stercoris, B. eggerthii, B. merdae, and B. caccae), Campylobacter spp. (including, for example, Campylobacter jejuni, Campylobacter laridis, and Campylobacter hyointestinalis), Clostridium spp. (such as the pathogenic clostridia including all types of Clostridium botulinum (including those in Groups I, II, III and IV, and including those that produce botulism A, B, C, D, E, F and G), all types of Clostridium tetani, all types of Clostridium difficile, and all types of Clostridium perfringens), Ebola spp. (e.g. EBOV Zaire), Enterobacter spp. (including, for example, Enterobacter aerogenes (also sometimes referred to as Klebsiella mobilis), Enterobacter agglomerans (also sometimes referred to as Pantoea agglomerans), Enterobacter amnigenus, Enterobacter asburiae, Enterobacter cancerogenus (also sometimes referred to as Enterobacter taylorae and/or Erwinia cancerogena), Enterobacter cloacae, Enterobacter cowanii, Enterobacter dissolvens (also sometimes referred to as Erwinia dissolvens), Enterobacter gergoviae, Enterobacter hormaechei, Enterobacter intermedium, Enterobacter intermedius (also sometimes referred to as Enterobacter intermedium), Enterobacter kobei, Enterobacter nimipressuralis (also sometimes referred to as Erwinia nimipressuralis), Enterobacter sakazakii, and Enterobacter taylorae (also sometimes referred to as Enterobacter cancerogenus)), Enterococcus spp. (including, for example, Vancomycin Resistant Enterococcus (VRE), Enterococcus faecalis, Enterococcus faecium, Enterococcus durans, Enterococcus gallinarum, and Enterococcus casseliflavus), Escherichia spp. (including the enterotoxigenic (ETEC) strains, the enteropathogenic (EPEC) strains, the enterohemorrhagic (EHEC) strain designated E. coli O157:H7, and the enteroinvasive (EIEC) strains), Gastrospirillum spp. (including, for example, Gastrospirillum hominis (also sometimes now referred to as Helicobacter heilmannii), Helicobacter spp. (including, for example, Helicobacter pylori and Helicobacter hepaticus), Klebsiella spp. (including, for example, Klebsiella pneumoniae, Klebsiella ozaenae, Klebsiella rhinoscleromatis, Klebsiella oxytoca, Klebsiella planticola, Klebsiella terrigena, and Klebsiella omithinolytica), Salmonella spp. (including, for example, S. typhi and S. paratyphi A, B, and C, S. enteritidis, and S. dublin), Shigella spp. (including, for example, Shigella sonnei, Shigella boydii, Shigella flexneri, and Shigella dysenteriae), Staphylococcus spp. (including, for example, Staphylococcus aureus, methicillin-resistant Staphylococcus aureus (MRSA), Staphylococcus saprophyticus and Staphylococcus epidermis), Streptococcus ssp. (including Groups A (one species with 40 antigenic types, Streptococcus pyogenes), B, C, D (five species (Streptococcus faecalis, Streptococcus faecium, Streptococcus durans, Streptococcus avium, and Streptococcus bovis)), F, and G, including Streptococcus pneumoniae), Pseudomonas spp. (including, for example, Pseudomonas aeruginosa, Pseudomonas maltophilia, Pseudomonas fluorescens, Pseudomonas putida, Pseudomonas cepacia, Pseudomonas stutzeri, Pseudomonas mallei, Pseudomonas pseudomallei and Pseudomonas putrefaciens), Vibrio spp. (including, for example, Vibrio cholera Serogroup O1 and Vibrio cholera Serogroup Non-O1, Vibrio parahaemolyticus, Vibrio alginolyticus, Vibrio furnissii, Vibrio carchariae, Vibrio hollisae, Vibrio cincinnatiensis, Vibrio metschnikovii, Vibrio damsela, Vibrio mimicus, Vibrio vulnificus, and Vibrio fluvialis), Yersinia spp. (including, for example, Yersinia pestis, Yersinia enterocolitica and Yersinia pseudotuberculosis), Neisseria, Proteus, Citrobacter, Aerobacter, Providencia, Serratia, Brucella, Francisella tularensis (also sometimes referred to as Pasteurella tularensis, Bacillus tularensis, Brucella tularensis, tularemia, rabbit fever, deerfly fever, Ohara's disease, and/or Francis disease), and the like.

Thus, for example, various bacterial infections or unwanted levels of bacteria that can be treated, prevented or addressed by the present inactivated agents include but are not limited to those associated with anthrax (Bacillus anthracis), staph infections (Staphylococcus aureus), typhus (Salmonella typhi), food poisoning (Escherichia coli, such as O157:H7), bascillary dysentery (Shigella dysenteria), pneumonia (Psuedomonas aerugenosa and/or Pseudomonas cepacia), cholera (Vibrio cholerae), ulcers (Helicobacter pylori), Bacillus cereus, Salmonella, Clostridium perfringens, Campylobacter, Listeria monocytogenes, Vibrio parahaemolyticus, botulism (Clostridium botulinum), smallpox (variola major), listeriosis (Listeria monocytogenes), tularemia (Francisella tularensis), plague (Yersinia pestis; also sometimes referred to as bubonic plague, pneumonic plague, and/or black death) and others. E. coli serotype O157:H7 has been implicated in the pathogenesis of diarrhea, hemorrhagic colitis, hemolytic uremic syndrome (HUS) and thrombotic thrombocytopenic purpura (TTP). As indicated herein, the internally inactivated agents of this invention are also active against drug-resistant and multiply-drug resistant strains of bacteria, for example, multiply-resistant strains of Staphylococcus aureus and vancomycin-resistant strains of Enterococcus faecium and Enterococcus faecalis.

Fungal infections that can be treated or prevented by the internally inactivated agents of this invention include infections by fungi that infect a mammal or a bird, including Histoplasma capsulatum, Coccidioides immitis, Cryptococcus neoformans, Candida ssp. including Candida albicans, Aspergilli ssp. including Aspergillus fumigatus, Sporothrix, Trichophyton ssp., Fusarium ssp., Tricosporon ssp., Pneumocystis carinii, and Trichophyton mentagrophytes. Hence, for example, infections or unwanted levels of target fungi can be treated, prevented or addressed by the present inactivated agents. Such fungi also include fungal pathogens that may have potential for use biological weapons, including Coccidioides immitis and Histoplasma capsulatum.

Prions that are treatable in the invention by the internally inactivated agents are proteins that can access multiple conformations, at least one of which is beta-sheet rich, infectious and self-perpetuating in nature. These infectious proteins show several remarkable biological activities, including the ability to form multiple infectious prion conformations, also known as strains or variants, encoding unique biological phenotypes, and to establish and overcome prion species (transmission) barriers. See, e.g., Tessier et al., Unraveling infectious structures, strain variants and species barriers for the yeast prion [PSI+], Nat. Struct. Mol. Biol. 2009 June; 16(6):598-605.

Cancers that can be treated by the internally inactivated agents of this invention include solid mammalian tumors as well as hematological malignancies. Solid mammalian tumors include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. Hematological malignancies include childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers. Both human and veterinary uses are contemplated.

Besides cancers, other disorders of the lymph nodes (such as for example viral and/or bacterial infections) can be treated by the internally inactivated agents of the invention. Accordingly, in one embodiment of the invention, there is provided a method for treating a subject with a virus or a bacterium which 1) provides within lymph nodes of the subject at least one photoactive or photoactivatable drug for treatment of the virus or the bacterium, and 2) applies initiation energy from at least one source to the lymph nodes. The method 3) activates directly or indirectly the at least one photoactive or photoactivatable drug at the target inside the lymph nodes. Inside the lymph nodes, the method 4) reacts the activated drug with the virus or bacterium to inactivate the virus or the bacterium to thereby treat the subject.

Pharmaceutical formulations containing the photoactivatable and energy modulation agents of the invention can be prepared by procedures known in the art using well-known and readily available ingredients. For example, the agents can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions can also contain thickening agents such as cellulose and/or cellulose derivatives. The compositions may also contain gums such as xanthan, guar or carbo gum or gum arabic, or alternatively polyethylene glycols, bentones and montmorillonites, and the like.

For example, tablets or caplets containing the photoactivatable and energy modulation agents of the invention can include buffering agents such as calcium carbonate, magnesium oxide and magnesium carbonate. Caplets and tablets can also include inactive ingredients such as cellulose, pre-gelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing at least one inactivated agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric-coated caplets or tablets containing one or more inactivated agents of the invention are designed to resist disintegration in the stomach and dissolve in the more neutral to alkaline environment of the duodenum.

The photoactivatable agents and energy modulation agents of the invention can also be formulated as elixirs or solutions for convenient oral administration or as solutions appropriate for parenteral administration, for instance by intramuscular, subcutaneous, intraperitoneal or intravenous routes. The pharmaceutical formulations of the photoactivatable and energy modulation agents of the invention can also take the form of an aqueous or anhydrous solution or dispersion, or alternatively the form of an emulsion or suspension or salve.

Thus, the photoactivatable agents and/or energy modulation agents may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelve life of the dosage form. The photoactivatable and energy modulation agents and other ingredients may form suspensions, solutions, or emulsions in oily or aqueous vehicles, and may contain formulatory agents such as suspending, stabilizing and/or dispersing agents. Alternatively, the photoactivatable and energy modulation agents and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C1-C4 alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if desired, an adjuvant chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

Also contemplated are combination products that include one or more of the photoactivatable agents and energy modulation agents of the invention and one or more other anti-microbial agents. For example, a variety of antibiotics can be included in the pharmaceutical compositions of the invention, such as aminoglycosides (e.g., streptomycin, gentamicin, sisomicin, tobramycin and amicacin), ansamycins (e.g. rifamycin), antimycotics (e.g. polyenes and benzofuran derivatives), β-lactams (e.g. penicillins and cephalosporins), chloramphenical (including thiamphenol and azidamphenicol), linosamides (lincomycin, clindamycin), macrolides (erythromycin, oleandomycin, spiramycin), polymyxins, bacitracins, tyrothycin, capreomycin, vancomycin, tetracyclines (including oxytetracycline, minocycline, doxycycline), phosphomycin and fusidic acid.

Additionally, the photoactivatable agents and/or energy modulation agents are well suited to formulation as sustained release dosage forms and the like. The formulations can be so constituted that they release the inactivated agent, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, draining devices and the like.

For topical administration, the photoactivatable agents and/or energy modulation agents may be formulated as is known in the art for direct application to a target area, for example the lymph nodes. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic agents of the invention (i.e., at least the photoactivatable agents and/or energy modulation agents) can be delivered via patches or bandages for dermal administration. Alternatively, the photoactivatable agents and/or energy modulation agents can be formulated to be part of an adhesive polymer, such as polyacrylate or acrylate/vinyl acetate copolymer. For long-term applications, in one embodiment of the invention, it might be desirable to use microporous and/or breathable backing laminates, so hydration or maceration of the skin can be minimized. The breathable backing layer can be any appropriate thickness that will provide the desired protective and support functions. A suitable thickness will generally be from about 10 to about 200 microns.

Ointments and creams containing the photoactivatable agents and/or energy modulation agents may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The photoactivatable agents and/or energy modulation agents can also be delivered via iontophoresis, e.g., as disclosed in U.S. Pat. Nos. 4,140,122; 4,383,529; or 4,051,842 (the contents of each are incorporated herein in their entirety). The percent by weight of the photoactivatable and energy modulation agents of the invention present in a topical formulation will depend on various factors, but generally will be from 0.01% to 95% of the total weight of the formulation, and typically 0.1-85% by weight.

Drops, such as eye drops or nose drops, may be formulated with one or more of the photoactivatable agents and/or energy modulation agents in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic photoactivatable agents and/or energy modulation agents and any other active ingredients may be formulated as a lozenge further comprising a flavored base, for example, sucrose and acacia or tragacanth; pastilles comprising the composition in an inert base such as gelatin and glycerin or sucrose and acacia; and mouthwashes comprising the composition of the invention in a suitable liquid carrier.

The pharmaceutical formulations of the invention containing the photoactivatable agents and/or energy modulation agents may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the invention include water and physiologically acceptable buffered saline solutions such as phosphate buffered saline solutions pH 7.0-8.0.

The photoactivatable agents and/or energy modulation agents of the invention can also be administered to the respiratory tract. Thus, the invention also provides aerosol pharmaceutical formulations and dosage forms for use in the invention. In general, such dosage forms comprise an amount of at least one of the photoactivatable and energy modulation agents of the invention effective to treat or prevent the clinical symptoms of a specific infection, cancer, tumor or disease. Any statistically significant attenuation of one or more symptoms of an infection, cancer, tumor or disease that has been treated pursuant to the methods of the invention is considered to be a treatment or prevention of such infection, cancer, tumor or disease within the scope of the invention. Accordingly, in one embodiment of the invention, there is provided a method for treating a subject carrying a virus or a bacterium which 1) provides within a respiratory track of the subject at least one photoactive or photoactivatable drug for treatment of a subject carrying the virus or the bacterium, and 2) applies initiation energy from at least one source to the respiratory track. The method 3) activates directly or indirectly the at least one photoactive or photoactivatable drug at the target inside the respiratory track. Inside the respiratory track, the method 4) reacts the activated drug with the virus or bacterium to inactivate the virus or the bacterium to thereby treat the subject.

Alternatively, for administration by inhalation or insufflation, the photoactivatable agents and/or energy modulation agents may be in a composition which takes the form of a dry powder, for example, a powder mix of the photoactivatable and energy modulation agents and a suitable powder base such as lactose or starch. The powder composition may be presented in unit dosage form in, for example, capsules or cartridges, or, e.g., gelatin or blister packs from which the powder may be administered with the aid of an inhalator, insufflator, or a metered-dose inhaler (see, for example, the pressurized metered dose inhaler (MDI) and the dry powder inhaler disclosed in Newman, S. P. in AEROSOLS AND THE LUNG, Clarke, S. W. and Davia, D. eds., pp. 197-224, Butterworths, London, England, 1984).

Therapeutic photoactivatable agents and/or energy modulation agents of the invention can also be administered in an aqueous solution when administered in an aerosol or inhaled form. Thus, other aerosol pharmaceutical formulations may comprise, for example, a physiologically acceptable buffered saline solution containing between about 0.1 mg/ml and about 100 mg/ml of one or more of the inactivated agents of the invention specific for the indication or disease to be treated or prevented. Dry aerosol in the form of finely divided solid inactivated agent that are not dissolved or suspended in a liquid are also useful in the practice of the invention. The photoactivatable agents and/or energy modulation agents of the invention may be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating or preventing the particular infection, indication or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the therapeutic photoactivatable agents and/or energy modulation agents of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627 (the contents of each are incorporated herein in their entirety). Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

Furthermore, the photoactivatable agents and/or energy modulation agents may also be used in combination with other therapeutic agents, for example, pain relievers, anti-inflammatory agents, antihistamines, bronchodilators and the like, whether for the conditions described or some other condition.

In various embodiments of this invention, a photoactive or photoactivatable drug which releases nitric oxide can be used to kill bacteria inside an organ is used separately or in conjunction with the alkylating agent or the psoralen noted above. For example, a photoactive manganese nitrosyl, namely [Mn(PaPy₃)(NO)](ClO₄) ({Mn—NO}) could be administered with the energy modulation agents of this invention. Activation of the energy modulation agents would result in the in vivo generation of visible or UV light which would induce local NO emission.

In one embodiment of this invention, the one or more wavelengths address factors which influence the progression of necrosis or its symptoms. In one embodiment of this invention, the different wavelengths provides for a more “programmed” apoptosis to eliminate unhealthy cells such as host cells containing a virus or bacteria cells or cancer cells. In one embodiment of this invention, the different wavelengths can promote interferon-beta which triggers cells to undergo necrosis.

Interferons (IFNs) are proteins made and released by host cells in response to the presence of pathogens such as viruses, bacteria, parasites or tumor cells. IFNs allow for communication between cells to trigger the protective defenses of the immune system that eradicate pathogens or tumors. IFNs belong to the large class of glycoproteins known as cytokines. Interferons are named after their ability to “interfere” with viral replication within host cells. IFNs have other functions: they activate immune cells, such as natural killer cells and macrophages; they increase recognition of infection or tumor cells by up-regulating antigen presentation to T lymphocytes.

In general, the one or more wavelengths provided to the target structure can selectively turn on different biological responses. However, the invention is not so limited and different wavelengths provided to the target structure may have a cumulative (or alternatively a synergistic) effect with regard to one or more biological response. Moreover, in the invention, the biological response may be one which suppresses, treats, or cures a biological condition. For example, energy modulation agents directed to the activation of psoralen and other energy modulation agents directed to the activation of an alkylating agent can be used simultaneously to treat tumors. For example, energy modulation agents directed to the activation of psoralen and other energy modulation agents directed to the activation of an alkylating agent can be used simultaneously to treat viral infections. For example, energy modulation agents directed to the activation of psoralen and other energy modulation agents directed to the activation of an alkylating agent can be used simultaneously to treat bacterial infections.

In various embodiments, different biological responses may include not only the activation of a drug but also the redirection of metabolic pathways, up-regulation of certain genes, down-regulation of certain genes, secretion of cytokines, alteration of cytokine receptor responses, or combinations thereof.

In various embodiments, a first biological response caused by light a first wavelength can result in the bonding of a drug or pharmaceutical agent to a critical cellular structure such as nuclear DNA, mRNA, rRNA, ribosome, mitochondrial DNA, or any other functionally important structures. A second biological response caused by light a second wavelength can result in releasing metabolites which can interfere with normal metabolic pathways. A second biological response caused by light a second wavelength can result in altering a targeted cellular response and/or other suitable biochemical or metabolic alterations. Increases in cerebral blood flow accompanied by a significant increase in nitric oxide production have been observed in subjects treated with low levels of 808 nm radiation.

Additionally, in one embodiment of this invention, one of the different wavelengths provided to the target structure can be at wavelengths of 620 nm, 680 nm, 760 nm, and 820-830 nm. Other suitable wavelengths (ranges) for photobiomodulation include 1) 613.5-623.5 nm, 2) 667.5-683.7 nm, 3) 750.7-772.3 nm, 4) 812.5-846.0 nm. These wavelengths (and the wavelengths described below) are useful in the invention as the different wavelengths provided to the target structure affect photobiomodulation.

For example, light in the far-red to near-IR spectral range (as one of the different wavelengths provided to a target structure) can modulate various biological processes by activation of mitochondrial respiratory chain components, resulting in initiation of a signaling cascade that promotes cellular proliferation and cytoprotection. Cytochrome oxidase is considered to be a key photoacceptor of light in the far-red to near-IR spectral range. Cytochrome oxidase is an integral membrane protein that contains four redox active metal centers and has a strong absorbency in the far-red to near-IR spectral range detectable in vivo by near-IR spectroscopy. Light at 660-680 nm of irradiation (as one of the different wavelengths provided to a target structure) can increase electron transfer in cytochrome oxidase, increase mitochondrial respiration and up-regulate cytochrome oxidase activity in neuronal cells.

Photostimulation can induce a cascade of signaling events initiated by the initial absorption of light by cytochrome oxidase. These signaling events may include the activation of immediate early genes, transcription factors, cytochrome oxidase subunit gene expression, and a host of other enzymes and pathways related to increased oxidative metabolism. Red to near-IR light stimulation (as one of the different wavelengths provided to a target structure) of mitochondrial electron transfer can increase the generation of reactive oxygen species. These mitochondrially generated reactive oxygen species may function as signaling molecules to provide communication between mitochondria and the cysts and nucleus.

Furthermore, in this photobiomodulation embodiment, one of the other wavelengths provided to the target structure can be in the ultraviolet range to induce activation of a photoactivatable drug such as a psoralen or an alkylating agent. In this embodiment, one of the other wavelengths provided to the target structure can be those wavelengths noted above which tend to reduce inflammation.

In another embodiment, one of the different wavelengths provided to the target structure can be in the range of 400 to 700 nm to reduce the degree of neointima formation and the incidence of restenosis (a narrowing of a blood vessel, leading to restricted blood flow). Restenosis is a common adverse event ofendovascular procedures such as vascular surgery, cardiac surgery, and angioplasty. Indeed, the phenomenon of vessel restenosis, an immune response to damaged tissue, is known to be a common adverse event and is one of the leading problems with angioplasty and stenting. Accordingly, in this embodiment, light in the range of 400 to 700 nm wavelength range, and more specifically in the 594-600 nm, can be provided as one of the different wavelengths provided to the target structure in vivo to decrease fibrointimal thickening following the arterial injury. In this embodiment, one of the other wavelengths provided to the target structure can be those wavelengths noted above which tend to reduce inflammation.

In another embodiment, one of the wavelengths provided to the target structure can be in the range of red to infrared for modulation of brain cell activity. In this embodiment, one of the different wavelengths provided to the target structure can be between 630 nm and 800 nm or 808 nm, in near-infrared spectrum or other wavelengths particularly suitable for transmission and dispersion within the gray matter and white matter of the brain. It has been shown that, within the visible and near-infrared spectral range, white matter in both the central and peripheral nervous systems reflects most of the incident power and shows a low level of absorption and a short penetration depth. In contrast, the transmittance of the gray matter is approximately twice as high as that of the white matter.

While, in the invention, the initiation energy (e.g., x-ray flux) can readily penetrate into the recessed areas of the brain to generate by way of energy modulation agents (down converters) near infrared light, generation of the near infrared light in these areas and propagation of near infrared light throughout the diseased cells of the brains is considered to be a highly beneficial aspect of this invention. For example, in this embodiment, exposure of the brain cells to these wavelengths in the near infrared can induce whole-brain metabolic and antioxidant beneficial effects such as increases in cytochrome oxidase and superoxide dismutase activities and increases in cerebral blood flow. Additionally, this treatment can include other drugs known to have a beneficial effect on brain disorders. Furthermore, in this embodiment, one of the other wavelengths provided to the target structure can be in the ultraviolet range to induce activation of a photoactivatable drug such as psoralen. In this embodiment, one of the other wavelengths provided to the target structure can be those wavelengths noted above which tend to reduce inflammation. (Although the description above is directed to brain disorders, these treatments according to this invention would be useful of the treatment of other neural conditions throughout the body.) In another embodiment of this invention, one of the different wavelengths provided to the target structure can be either a yellow or a green light. As noted above, photostimulation can be used to activate a light-sensitive protein such as rhodopsin (ChR2), which can then excite the cell expressing the opsin. It has been shown that channelrhodopsin-2, a monolithic protein containing a light sensor and a cation channel, provides electrical stimulation of appropriate speed and magnitude to activate neuronal spike firing. Thus, light-sensitive proteins can be introduced into cells or live subjects via a number of techniques including electroporation, DNA microinjection, viral delivery, liposomal transfection and calcium-phosphate precipitation. The gene, chloride pump (NpHR), which is borrowed from a microbe called an archaebacterium, can make neurons less active in the presence of yellow light. By combining genes ChR2 and NpHR, neurons can be made to obey pulses of light like drivers obey a traffic signal: Blue means “go” (emit a signal), and yellow means “stop” (don't emit). Accordingly, a light-sensitive protein (for example, channelrhodopsin-2 (ChR2) and chloride pump halorhodopsin (NpHR)) can be incorporated into the lentiviral vector or other vector providing delivery of the light-sensitive protein encoding gene into a target cell. ChR2 containing a light sensor and a cation channel, provides electrical stimulation of appropriate speed and magnitude to activate neuronal spike firing, when the cells harboring Ch2R are pulsed with light. Thus, in the invention, the photoactivation can lead to either suppression or activation of a biological process depending on the gene selected and the wavelength of light chosen. Furthermore, in this embodiment, one of the other wavelengths provided to the target structure can be in the ultraviolet range to induce activation of a photoactivatable drug such as a psoralen or an alkylating agent. In this embodiment, one of the other wavelengths provided to the target structure can be those wavelengths noted above which tend to reduce inflammation.

In another embodiment of this invention, one of the different wavelengths provided to the target structure can be 632 nm light for generation of a light-oxygen effect (LOE), which involves activation of or damage to biosystems by optical radiation at low optical doses by direct photoexcitation of molecular oxygen dissolved in a biosystem so that oxygen dissolved is converted to a singlet state, i.e., by photogeneration of molecular singlet oxygen from O₂ dissolved in cells. This process can occur in the presence or absence of a photosensitizer. Furthermore, in this embodiment, one of the other wavelengths provided to the target structure can be in the ultraviolet range to induce activation of a photoactivatable drug such as psoralen. In this embodiment, one of the other wavelengths provided to the target structure can be those wavelengths noted above which tend to reduce inflammation.

While denoted above as belonging to either first or second biological responses, the invention can affect any of the biological responses set forth in this specification as either a first or a second biological response. Furthermore, the sequence of biological responses does not necessarily follow in order. For example, a first biological response could in time actually occur first or second or simultaneously. Likewise, a second biological response could actually occur in time as a first response or a second response or simultaneously with the first biological response.

In one embodiment, the method applies initiation energy from at least one source to the target structure, wherein the initiation energy contacts the target structure, generates at least one or more different wavelengths of light, and induces a predetermined change in the target structure in situ.

In one embodiment, the predetermined change modifies the target structure or modulates the biological activity of the target structure. In one embodiment, the predetermined change activates a photoactive or photoactivatable drug to treat a virus or viral infection or bacteria infection.

In one embodiment, the method contacts a target structure with at least one activatable pharmaceutical agent (PA) (e.g., a or photoactivatable drug) that is capable of effecting a predetermined change in the target structure when activated by the one or more different wavelengths of light one of a plurality of light emitters.

In one embodiment, the method applies an initiation energy from at least one source to a subject in need of treatment, wherein the initiation energy induces a predetermined change in the subject in situ (indirectly) by way of the selective activation from the plurality of light emitters at different wavelengths or (directly) by direct activation of the photoactive or photoactivatable drug. In one embodiment of the method, the predetermined change modifies modulates the biological activity of the target structure.

In one embodiment, the method contacts a target structure with at least one activatable pharmaceutical agent (PA) (e.g., a photoactivatable drug) that is capable of effecting a predetermined change in a target structure when activated by one of the plurality of light emitters, optionally in the presence of at least one member selected from the group consisting of energy modulation agents, plasmonics-active agents and combinations thereof.

In one embodiment, the energy modulation agent, if present, upgrades or downgrades the initiation energy to produce one or more of the different wavelengths of light. In one embodiment, the plasmonics-active agent, if present, enhances or modifies the light generated internally or provided internally within the subject or the applied initiation energy or both.

A further embodiment of the invention is to provide such methods which can use any suitable energy source as the initiation energy source in combination with plasmonics materials to activate the activatable pharmaceutical agent and thereby cause the predetermined change.

A further embodiment of the invention is to provide such methods using plasmonics in an energy cascade to activate an activatable pharmaceutical agent that then cause the predetermined change.

A further embodiment of the invention is to provide such methods for in situ generation of energy which causes, either directly or indirectly, the predetermined change.

A further embodiment of the invention is to provide a method for the treatment of a cell proliferation disorder that permits treatment of a subject in any area of the body while being non-invasive and having high selectivity for targeted cells relative to healthy cells through the use of exciton-plasmon enhancement.

A further embodiment of the invention is to provide a method for treatment of a condition, disorder, or disease (preferably but not limited to viral or bacterial infections) which can use any suitable energy source as the initiation energy source in combination with plasmon enhancement to activate the activatable pharmaceutical agent.

A further embodiment of the invention is to provide a method for treatment of a condition, disorder or disease using plasmon enhancement in an energy cascade to activate an activatable pharmaceutical agent that then treats cells suffering from a condition, disorder or disease (preferably but not limited to viral or bacterial infections).

The condition, disorder, or disease may be mediated by abnormal cellular proliferation and the predetermined change in one embodiment can ameliorate the abnormal cellular proliferation. Abnormal cellular proliferation may be higher than that of cells from a subject not having said condition, disorder or disease or may be lower.

The treated condition, disorder, or disease may or may not be significantly mediated by abnormal cellular proliferation, and the predetermined change does not have to substantially affect cellular proliferation.

The target structure need not be present inside an organism, but may be one in vitro or c; vivo. The predetermined change may enhance the expression of, promote the growth of, or increase the quantity of the target structure; or the predetermined change can enhance, inhibit or stabilize the usual biological activity of the target structure compared to a similar untreated target structure. For example, the predetermined change can alter the immunological or chemical properties of the target structure which may be a cell, cell membrane, internal cellular structure, polypeptide or non-polypeptide compound which can be modified by said predetermined change to be more or less antigenic or immunogenic. In another embodiment, modifying the target structure can be done without the need for a pharmaceutical agent, or a plasmonics agent.

One embodiment of the invention is to modify a target structure which mediates or is associated with a biological activity, and in a preferred embodiment to treat a condition, disorder or disease, (preferably but not limited to viral or bacterial infections) in a subject using photobiomodulation by way of the selective activation noted above.

Accordingly, exemplary conditions, disorders or diseases may include, but are not limited to, cancer, autoimmune diseases, chronic pain, wound healing, nerve regeneration, viral and bacterial infections, perceptional and cognitive disorders. Exemplary conditions also may include nerve (brain) imaging and stimulation, control of cell death (apoptosis), and alteration of cell growth and division. Other exemplary conditions, disorders or diseases may include, but are not limited to cardiac ablasion (e.g., cardiac arrhythmia and atrial fibrillation), photoangioplastic conditions (e.g., de novo atherosclerosis, restinosis), intimal hyperplasia, arteriovenous fistula, and macular degeneration, or others conditions exacerbated by the viral or bacterial infection.

A method in accordance with one embodiment of the invention utilizes the principle of energy transfer to and among molecular agents to control delivery and activation of cellular changes by irradiation such that delivery of the desired effect is more intensified, precise, and effective than the conventional techniques. At least one energy modulation agent can be administered to the subject which adsorbs, intensifies or modifies said initiation energy into an energy that effects a predetermined cellular change in the target structure. The energy modulation agent may be located around, on, or in the target structure. Further, the energy modulation agent can transform a photonic initiation energy into a photonic energy that effects a predetermined change in the target structure. In one embodiment, the energy modulation agent decreases the wavelength of the photonic initiation energy (down convert). In another embodiment, the energy modulation agent can increase the wavelength of the photonic initiation energy (up convert). In a different embodiment the modulation agent is one or more members selected from a biocompatible fluorescing metal nanoparticle, fluorescing metal oxide nanoparticle, fluorescing dye molecule, gold nanoparticle, silver nanoparticle, gold-coated silver nanoparticle, a water soluble quantum dot encapsulated by polyamidoamine dendrimers, a luciferase, a biocompatible phosphorescent molecule, a combined electromagnetic energy harvester molecule, and a lanthanide chelate exhibiting intense luminescence.

In one aspect of the invention, a downconverting energy modulation agent can comprise inorganic particulates selected from the group consisting of: metal oxides; metal sulfides; doped metal oxides; and mixed metal chalcogenides. In one aspect of the invention, the downconverting material can comprise at least one of Y₂O₃, Y₂O₂S, NaYF₄, NaYbF₄, YAG, YAP, Nd₂O₃, LaF₃, LaCl₃, La₂O₃, TiO₂, LuPO₄, YVO₄, YbF₃, YF₃, Na-doped YbF₃, ZnS; ZnSe; MgS; CaS; CaWO₄, CaSiO₂:Pb, and alkali lead silicate including compositions of SiO₂, B₂O₃, Na₂O, K₂O, PbO, MgO, or Ag, and combinations or alloys or layers thereof. In one aspect of the invention, the downconverting material can include a dopant including at least one of Er, Eu, Yb, Tm, Nd, Mn Tb, Ce, Y, U, Pr, La, Gd and other rare-earth species or a combination thereof. The dopant can be included at a concentration of 0.01%-50% by mol concentration.

In one aspect of the invention, the downconverting energy modulation agent can comprise materials such as ZnSeS:Cu, Ag, Ce, Tb; CaS: Ce,Sm; La₂O₂S:Tb; Y₂O₂S:Tb; Gd₂O₂S:Pr, Ce, F; LaPO₄. In other aspects of the invention, the downconverting material can comprise phosphors such as ZnS:Ag and ZnS:Cu, Pb. In other aspects of the invention, the downconverting material can be alloys of the ZnSeS family doped with other metals. For example, suitable materials include ZnSeₓS_{y}:Cu, Ag, Ce, Tb, where the following x, y values and intermediate values are acceptable: x:y; respectively 0:1; 0.1:0.9; 0.2:0.8; 0.3:0.7; 0.4:0.6; 0.5:0.5; 0.6:0.4; 0.7:0.3; 0.8:0.2; 0.9:0.1; and 1.0:0.0.

In other aspects of the invention, the downconverting energy modulation agent can be materials such as sodium yttrium fluoride (NaYF₄), lanthanum fluoride (LaF₃), lanthanum oxysulfide (La₂O₂S), yttrium oxysulfide (Y₂O₂S), yttrium fluoride (YF₃), yttrium gallate, yttrium aluminum garnet (YAG), gadolinium fluoride (GdF₃), barium yttrium fluoride (BaYF₅, BaY₂F₈), gadolinium oxysulfide (Gd₂O₂S), calcium tungstate (CaWO₄), yttrium oxide:terbium (Yt₂O₃Tb), gadolinium oxysulphide:europium (Gd₂O₂S:Eu), lanthanum oxysulphide:europium (La₂O₂S:Eu), and gadolinium oxysulphide:promethium, cerium, fluorine (Gd₂O₂S:Pr,Ce,F), YPO₄:Nd, LaPO₄:Pr, (Ca,Mg)SO₄:Pb, YBO₃:Pr, Y₂SiO₅:Pr, Y₂Si₂O₇:Pr, SrLi₂SiO₄:Pr,Na, and CaLi₂SiO₄:Pr.

In other aspects of the invention, the downconverting energy modulation agent can be near-infrared (NIR) downconversion (DC) phosphors such as KSrPO₄:Eu²⁺, Pr³⁺, or NaGdF₄:Eu or Zn₂SiO₄:Tb³⁺,Yb³⁺ or —NaGdF₄ co-doped with Ce⁺ and Tb³⁺ ions or Gd₂O₂S:Tm or BaYF₅:Eu³⁺ or other down converters which emit NIR from visible or UV light exposure (as in a cascade from x-ray to UV to NIR) or which emit NIR directly after x-ray or e-beam exposure.

In one aspect of the invention, an up-converting energy modulation agent can be at least one of Y₂O₃, Y₂O₂S, NaYF₄, NaYbF₄, YAG, YAP, Nd₂O₃, LaF₃, LaCl₃, La₂O₃, TiO₂, LuPO₄, YVO₄, YbF₃, YF₃, Na-doped YbF₃, or SiO₂ or alloys or layers thereof.

In one aspect of the invention, the energy modulation agents can be used singly or in combination with other down converting or up converting materials.

In one embodiment of the invention, the present invention utilizes a novel phosphor-containing drug activator for causing a change in activity in a subject that is effective, specific, and able to produce a change to the medium or body. The phosphor-containing drug activator comprises a mixture of two different ones of the energy modulation agents described above, The energy modulation agents, which upon x-ray excitation, each have emissions in the UV and/or visible spectrum. The mixture of energy modulation agents results in superior performance compared to either phosphor alone. In one embodiment of the invention, the mixture is a mixture of phosphors preferably including a mixture of two or more phosphors, namely NP-200 and GTP-4300, that are purchased from Nichia and Global Tungsten and Powders, respectively. The chemical formulas of these phosphors are Zn₂SiO₄:Mn²⁺ and (3Ca₃(PO₄)₂Ca(F, Cl)₂: Sb³⁺, Mn²⁺), respectively. These phosphors absorb penetrating forms of energy (e.g., low dose x-rays) and emit light in wavelengths that activate a photoactivatable drug in-situ.

In one embodiment of the invention, the phosphor-containing drug activator includes an admixture or suspension of two or more phosphors capable of emitting ultraviolet and visible light upon interaction with x-rays; the two or more phosphors include Zn₂SiO₄:Mn² and (3Ca₃(PO₄)₂Ca(F, Cl)₂: Sb³⁺, Mn²⁺) at a ratio from 1:10 to 10. Each of said two phosphors has at least one coating selected from the group consisting of an ethylene cellulose coating and a diamond-like carbon coating; and, optionally in the case of the suspension, a pharmaceutically acceptable carrier. In one embodiment of the invention, the ratio ranges from 1:5 to 5:1, or from 1:2 to 2:1, or is about 2:1.

In one embodiment of the invention, the phosphors in the novel phosphor-containing drug activator are coated with a biocompatible Ethyl Cellulose coating and/or coated with a Diamond Like Carbon (DLC) coatings.

In one embodiment, the phosphors in the phosphor-containing drug activator are first coated with a biocompatible Ethyl Cellulose coating, and then overcoated with a second coating of Diamond Like Carbon (DLC).

Ethyl Cellulose (EC) is widely used in biomedical applications today, including artificial kidney membranes, coating materials for drugs, blood coagulants, additives of pharmaceutical products, blood compatible materials. EC and its derivatives have been widely used in various, personal care, food, biomedical and drug related applications. EC is not a skin sensitizer, it is not an irritant to the skin, and it is not mutagenic. EC is generally regarded as safe (GRAS), and widely used for example in food applications such flavor encapsulation, inks for making fruits and vegetables, paper and paperboard in contact with aqueous and fatty foods.

EC is also widely used for controlled release of active ingredients. The enhanced lipophilic and hydrophobic properties make it a material of choice for water resistant applications. EC is soluble in various organic solvents and can form a film on surfaces and around particles (such as phosphors). In one embodiment of this invention, ethyl cellulose is used to encapsulate the phosphors particles of the phosphor-containing drug activator to ensure that an added degree of protection is in place on the surface of the phosphors. In one embodiment of this invention, EC polymers with high molecular weight for permanent encapsulation and long term biocompatibility are used to encapsulate the phosphors particles of the phosphor-containing drug activator. Diamond Like Carbon (DLC) films are in general dense, mechanically hard, smooth, impervious, abrasion resistant, chemically inert, and resistant to attack by both acids and bases; they have a low coefficient of friction, low wear rate, are biocompatible and thromboresistant. Tissues adhere well to carbon coated implants and sustain a durable interface. In presence of blood, a protein layer is formed which prevents the formation of blood clots at the carbon surface. For medical prostheses that contact blood (heart valves, anathomic sheets, stents, blood vessels, etc.), DLC coatings have been used.

DLC has emerged over the past decade as a versatile and useful biomaterial.

It is harder than most ceramics, bio-inert, and has a low friction coefficient. DLC is one of the best materials for implantable applications. Studies of the biocompatibility of DLC demonstrate that there is no cytotoxicity and cell growth is normal on a DLC-coated surface. (DLC coatings on stainless steel have performed very well in in vitro studies of hemocompatibility. Histopathological investigations have shown good biotolerance of implants coated with the DLC. Moreover, DLC as a coating is efficient protection against corrosion. These properties make the embodiment described here with a double coating (EC and DLC) particularly advantageous for a phosphor-containing drug activator of the invention.

In one embodiment, the initiation energy can be provided by way of catheters inserted into the subject. The catheters can include prescribed energy modulation agents (e.g., up converting or down converting materials noted herein) at a distal end of the catheter which emit the aforementioned light for selective activation into the subject. In one embodiment, the catheters could directly provide light of a suitable wavelength to directly activate the photoactive drugs in vivo. This approach would be particularly attractive if an organ such as the liver (containing a substantial amount of blood was to be treated. This approach would “simulate” an extracorporeal photopheresis (ECP) treatment. In some embodiments, it may be possible to activate the photoactive drug directly with UV light being passed through the skin into a near surface vein or artery or for example through the mucosa of the tongue. While care must be exercised not to “burn” the skin or mucosa, photoactivation of the drug and viral arrest may produce enough inactivated viruses for the human body to induce an autovaccine response.

Regarding down conversion, below is a list of X-ray phosphors which can be used in the invention along with their corresponding peak emission values.

TABLE 1

| | | Emission | | | | | | |
|---|---|---|---|---|---|---|---|---|
| | | Spectrum | X-ray Absorption | | | Microstructure | | |
| | | Peak Emission | Emiss Eff | Eff | K-edge | Specific | Crystal | |
| # | Phosphor | (nm) | (%) | (Z) | (keV) | Gravity | Structure | Hygroscopic |
| 1 | BaFCl: Eu²⁺ | 380 | 13 | 49.3 | 37.38 | 4.7 | Tetragonal | N |
| 2 | BaSO₄—: Eu²⁺ | 390 | 6 | 45.5 | 37.38 | 4.5 | Rhombic | N |
| 3 | LaOBr: Tm³⁺ | 360, 460 | 14 | 49.3 | 38.92 | 6.3 | Tetragonal | N |
| 4 | YTaO₄ | 337 | | 59.8 | 67.42 | 7.5 | Monolithic | N |
| 5 | YTaO₄: Nb (*) | 410 | 11 | 59.8 | 67.42 | 7.5 | Monolithic | N |
| 6 | CaWO₄ | 420 | 5 | 61.8 | 69.48 | 6.1 | Tetragonal | N |
| 7 | LaOBr: Tb³⁺ | 420 | 20 | 49.3 | 38.92 | 6.3 | Tetragonal | N |
| 8 | Y₂O₂S: Tb³⁺ | 420 | 18 | 34.9 | 17.04 | 4.9 | Hexgonal | N |
| 9 | ZnS: Ag | 450 | 17 | 26.7 | 9.66 | 3.9 | Hexgonal | N |
| 10 | (Zn,Cd)S: Ag | 530 | 19 | 38.4 | 9.66/26.7 | 4.8 | Hexgonal | N |
| 11 | Gd₂O₂S: Tb³⁺ | 545 | 13 | 59.5 | 50.22 | 7.3 | Hexgonal | N |
| 12 | La₂O₂S: Tb³⁺ | 545 | 12.5 | 52.6 | 38.92 | 6.5 | Hexgonal | N |

Various plastic scintillators, plastic scintillator fibers and related materials are made of polyvinyltoluene or styrene and fluors. These materials could be used in the invention especially if encapsulated or otherwise chemically isolated from the target structure so not as to be dissolved or otherwise deteriorated by the fluids of the target structure. These and other formulations are commercially available, such as from Saint Gobain Crystals, as BC-414, BC-420, BC-422, or BCF-10.

TABLE 2

| Phosphor | Product Reference | Peak Emission (nm) |
|---|---|---|
| Organic | BC-414 | 392 |
| Organic | BC-420 | 391 |
| Organic | BC-422 | 370 |

Other polymers are able to emit in the visible range and these include:

TABLE 3

| Phosphor | | | # of Photons |
|---|---|---|---|
| (Fiber Forms) | Product Reference | Peak Emission (nm) | Per MeV |
| Organic | BCF-10 | 432 | 8000 |
| Organic | BC-420 | 435 | 8000 |
| Organic | BC-422 | 492 | 8000 |

Table 4 shows a wide variety of energy modulation agents which can be used in this invention.

TABLE 4

| | Emission | | | | | | |
|---|---|---|---|---|---|---|---|
| | Spectrum | X-Ray Absorption | | | | | |
| Phosphor | Peak Emission | Emiss Eff | Eff | K-edge | Specific | Crystal | |
| Color | (nm) | (%) | (Z) | (keV) | Gravity | Structure | Hygroscopic |
| Zn3(PO4)2: Tl+ | 310 | | | | | | N |
| BaF2 | 310 | | | | | | Slightly |
| CsI | 315 | | | | | | N |
| Ca3(PO4)2: Tl+ | 330 | | | | | | N |
| YTaO4 | 337 | | 59.8 | 67.42 | 7.5 | Monolithic | N |
| CsI: Na | 338 | | | | | | Y |
| BaSi2O5: Pb2+ | 350 | | | | | | N |
| Borosilicate | 350 | | | | | | N |
| LaCl3(Ce) | 350 | | | | | | Y |
| SrB4O7F: Eu2+ | 360 | | | | | | N |
| RbBr: Tl+ | 360 | | | | | | ? |
| (Ba, Sr, Mg)3Si2O7: Pb2+ | 370 | | | | | | N |
| YAlO3: Ce3+ | 370 | | | | | | N |
| BC-422 | 370 | | | | | Organic | ? |
| BaFCl: Eu2+ | 380 | 13 | 49.3 | 37.38 | 4.7 | Tetragonal | N |
| BaSO4—: Eu2+ | 390 | 6 | 45.5 | 37.38 | 4.5 | Rhombic | N |
| BaFBr: Eu2+ | 390 | | | | | | ? |
| BC-420 | 391 | | | | | Organic | ? |
| BC-414 | 392 | | | | | Organic | ? |
| SrMgP2O7: Eu2+ | 394 | | | | | | N |
| BaBr2: Eu2+ | 400 | | | | | | N |
| (Sr, Ba)Al2Si2O8: Eu2+ | 400 | | | | | | N |
| YTaO4: Nb (*) | 410 | 11 | 59.8 | 67.42 | 7.5 | Monolithic | N |
| Y2SiO5: Ce3+ | 410 | | | | | | N |
| CaWO4 | 420 | 5 | 61.8 | 69.48 | 6.1 | Tetragonal | N |
| LaOBr: Tb3+ | 420 | 20 | 49.3 | 38.92 | 6.3 | Tetragonal | N |
| Y2O25: Tb3+ | 420 | 18 | 34.9 | 17.04 | 4.9 | Hexgonal | N |
| Lu2SiO5: Ce3+ | 420 | | | | | | N |
| Lu1.8 Y0.2SiO5: Ce | 420 | | | | | | N |
| ZnS: Ag | 450 | 17 | 26.7 | 9.66 | 3.9 | Hexgonal | N |
| CdWO4 | 475 | | | | | | Slightly |
| Bi4Ge3O12 (BGO) | 480 | | | | | | N |
| (Zn, Cd)S: Ag | 530 | 19 | 38.4 | 9.66/26.7 | 4.8 | Hexgonal | N |
| Gd2O2S: Tb3+ | 545 | 13 | 59.5 | 50.22 | 7.3 | Hexgonal | N |
| La2O2S: Tb3+ | 545 | 12.5 | 52.6 | 38.92 | 6.5 | Hexgonal | N |
| Y3Al5O12 (Ce) | 550 | | | | | | N |
| LaOBr: Tm3+ | 360, 460 | 14 | 49.3 | 38.92 | 6.3 | Tetragonal | N |
| CaF2(Eu) | 435/300 | | | | | | N |

Selection of one or more phosphors depends on the reactivity of the photoactive or photoactivatable drug to the emitted light from the phosphor. As noted above, UV light in the range from 310 to 360 nm can activate an alkylating agent such as iodonophthylazide (INA). Other alkylating agents may need light of a different wavelength or a combination of wavelengths to be excited.

By selection of one or more of the phosphors noted above (or others known in the art), the invention permits one to provide in a vicinity of or within a target structure one or more light emitters capable of emitting different wavelengths corresponding to respective biological responses, and permits the activation of one or more biological responses in the target structure depending on at least one or more different wavelengths of light generated internally or provided internally within the subject, wherein the different wavelengths activate the respective biological responses (i.e., selective activation of different photoreactive drugs).

In one embodiment, the invention provides methods utilizing the principle of energy transfer to and among molecular agents to control delivery and activation of pharmaceutically active agents (e.g., psoralens or alkylating agents) such that delivery of the desired pharmacological effect is focused and precise.

In one embodiment, the initiation energy source is applied directly or indirectly (via a modulation agent) to the activatable pharmaceutical agent, preferably in proximity to the target cells.

Within the context of the invention, the phrase “applied indirectly” (or variants of this phrase, such as “applying indirectly”, “indirectly applies”, “indirectly applied”, “indirectly applying”, etc.), when referring to the application of the initiation energy, means the penetration by the initiation energy into the subject beneath the surface of the subject and to the modulation agent and/or activatable pharmaceutical agent within a subject. In one embodiment, the initiation energy interacts with a previously administered energy modulation agent which then activates the predetermined cellular changes. In another embodiment, the initiation energy interacts with a previously administered energy modulation agent which then activates the activatable pharmaceutical agent. In another embodiment, the initiation energy itself activates the activatable pharmaceutical agent. In either embodiment, the initiation energy source cannot be within line-of-sight of the modulation agent and/or the activatable pharmaceutical agent. By “cannot be within line-of-sight” is meant that if a hypothetical observer were located at the location of the modulation agent or the activatable pharmaceutical agent, that observer would be unable to see the source of the initiation energy.

Although not intending to be bound by any particular theory or be otherwise limited in any way, the following theoretical discussion of scientific principles and definitions are provided to help the reader gain an understanding and appreciation of the invention.

As used herein, the term “subject” is not intended to be limited to humans, but may also include animals, birds, plants, or any suitable biological organism.

As used herein, the phrase “a disease or condition” refers to a condition, disorder or disease that may include, but are not limited to, cancer, soft and bone tissue injury, chronic pain, wound healing, nerve regeneration, viral and bacterial infections, fat deposits (liposuction), varicose veins, enlarged prostate, retinal injuries and other ocular diseases, Parkinson's disease, and behavioral, perceptional and cognitive disorders.

As used herein, the term “target structure” refers to an eukaryotic cell, prokaryotic cell, a subcellular structure, such as a cell membrane, a nuclear membrane, cell nucleus, nucleic acid, mitochondria, ribosome, or other cellular organelle or component, an extracellular structure, virus or prion, and combinations thereof.

The nature of the predetermined cellular change will depend on the desired pharmaceutical outcome. Exemplary cellular changes may include, but are not limited to, apoptosis, necrosis, up-regulation of certain genes, down-regulation of certain genes, secretion of cytokines, alteration of cytokine receptor responses, regulation of cytochrome c oxidase and flavoproteins, activation of mitochondria, stimulation antioxidant protective pathway, modulation of cell growth and division, alteration of firing pattern of nerves, alteration of redox properties, generation of reactive oxygen species, modulation of the activity, quantity, or number of intracellular components in a cell, modulation of the activity, quantity, or number of extracellular components produced by, excreted by, or associated with a cell, an immune response, an autovaccine response, or a combination thereof. Predetermined cellular changes may or may not result in destruction or inactivation of the target structure.

As used herein, an “energy modulation agent” refers to an agent that is capable of receiving an energy input from a source and then re-emitting a different energy to a receiving target. Energy transfer among molecules may occur in a number of ways. The form of energy may be electronic, thermal, electromagnetic, kinetic, or chemical in nature. Energy may be transferred from one molecule to another (intermolecular transfer) or from one part of a molecule to another part of the same molecule (intramolecular transfer). For example, a modulation agent may receive electromagnetic energy and re-emit the energy in the form of thermal energy. In preferred embodiments, the energy modulation agent receives higher energy (e.g. x-ray) and re-emits in lower energy (e.g. UV-A). Some modulation agents may have a very short energy retention time (on the order of fs, e.g. fluorescent molecules) whereas others may have a very long half-life (on the order of minutes to hours, e.g. luminescent or phosphorescent molecules). Suitable energy modulation agents include, but are not limited to, a biocompatible fluorescing metal nanoparticle, fluorescing dye molecule, gold nanoparticle, a water soluble quantum dot encapsulated by polyamidoamine dendrimers, a luciferase, a biocompatible phosphorescent molecule, a combined electromagnetic energy harvester molecule, and a lanthanide chelate capable of intense luminescence. Various exemplary uses of these are described below in preferred embodiments.

The modulation agents may further be coupled to a carrier for cellular targeting purposes. For example, a biocompatible molecule, such as a fluorescing metal nanoparticle or fluorescing dye molecule that emits in the UV-A band, may be selected as the energy modulation agent.

The energy modulation agent (or agents) may be preferably directed to the desired site (e.g. a tumor or a specific organ) by systemic administration to a subject.

For example, a UV-A emitting energy modulation agent (or agents) may be concentrated in the tumor site by physical insertion or by conjugating the UV-A emitting energy modulation agent with a tumor specific carrier, such as a lipid, chitin or chitin-derivative, a chelate or other functionalized carrier that is capable of concentrating the UV-A emitting source in a specific target tumor.

In one embodiment, the energy modulation agent can be used alone or as a series of two or more energy modulation agents wherein the energy modulation agents provide an energy cascade. Thus, the first energy modulation agent in the cascade will absorb the activation energy, convert it to a different energy which is then absorbed by the second energy modulation in the cascade, and so forth until the end of the cascade is reached with the final energy modulation agent in the cascade emitting the energy necessary to activate the activatable pharmaceutical agent.

As used herein, an “activatable pharmaceutical agent” is an agent that normally exists in an inactive state in the absence of an activation signal. When the agent is activated by a matching activation signal under activating conditions, it is capable of effecting the desired pharmacological effect on a target cell (i.e. preferably a predetermined cellular change).

Signals that may be used to activate a corresponding agent may include, but are not limited to, photons of specific wavelengths (e.g. x-rays, or visible light), electromagnetic energy (e.g. radio or microwave), thermal energy, acoustic energy, or any combination thereof.

Activation of the agent (or agents) may be as simple as delivering the signal to the agent or may further premise on a set of activation conditions. For example, in the former case, an activatable pharmaceutical agent, such as a photosensitizer, may be activated by UV-A radiation. Once activated, the agent (or agents) in its (their) active-state may then directly proceed to effect a cellular change.

Where activation may further premise upon other conditions, mere delivery of the activation signal may not be sufficient to bring about the desired cellular change.

For example, a photoactive compound that achieves its pharmaceutical effect by binding to certain cellular structure in its active state may require physical proximity to the target cellular structure when the activation signal is delivered. For such activatable agents, delivery of the activation signal under non-activating conditions will not result in the desired pharmacologic effect. Some examples of activating conditions may include, but are not limited to, temperature, pH, location, state of the cell, presence or absence of co-factors.

Selection of an activatable pharmaceutical agent greatly depends on a number of factors such as the desired cellular change, the desired form of activation, as well as the physical and biochemical constraints that may apply. Exemplary activatable pharmaceutical agents may include, but are not limited to, agents that may be activated by photonic energy, electromagnetic energy, acoustic energy, chemical or enzymatic reactions, thermal energy, or any other suitable activation mechanisms.

When activated, the activatable pharmaceutical agent may effect cellular changes that include, but are not limited to, apoptosis, redirection of metabolic pathways, up-regulation of certain genes, down-regulation of certain genes, secretion of cytokines, alteration of cytokine receptor responses, or combinations thereof.

The mechanisms by which an activatable pharmaceutical agent may achieve its desired effect are not particularly limited. Such mechanisms may include direct action on a predetermined target as well as indirect actions via alterations to the biochemical pathways. A preferred direct action mechanism is by binding the agent to a critical cellular structure such as nuclear DNA, mRNA, rRNA, ribosome, mitochondrial DNA, or any other functionally important structures. Indirect mechanisms may include releasing metabolites upon activation to interfere with normal metabolic pathways, releasing chemical signals (e.g. agonists or antagonists) upon activation to alter the targeted cellular response, and other suitable biochemical or metabolic alterations.

The treatment of the invention can be by the methods described in U.S. application Ser. No. 11/935,655, filed Nov. 6, 2007 (incorporated by reference above), or by a modified version of a conventional treatment such as PDT, but using a plasmonics-active agent to enhance the treatment by modifying or enhancing the applied energy or, in the case of using an energy modulation agent, modifying either the applied energy, the emitted energy from the energy modulation agent, or both.

In one preferred embodiment, the activatable pharmaceutical agent is capable of chemically binding to the DNA or mitochondria or across lipid bilayers of a virus at a therapeutically effective amount. In this embodiment, the activatable pharmaceutical agent, preferably a photoactivatable agent, is exposed in situ to an activating energy emitted from an energy modulation agent (or agents), which had received energy from an initiation energy source.

Suitable activatable agents include, but are not limited to, photoactive or photoactivatable agents, sono-active agents, thermo-active agents, and radio/microwave-active agents. An activatable agent may be a small molecule; a biological molecule such as a protein, a nucleic acid or lipid; a supramolecular assembly; a nanoparticle; or any other molecular entity having a pharmaceutical activity once activated.

The activatable agent may be derived from a natural or synthetic origin. Any such molecular entity that may be activated by a suitable activation signal source to effect a predetermined cellular change may be advantageously employed in the invention.

Suitable photoactive or photoactivatable agents include, but are not limited to: alkylating agents, psoralens and psoralen derivatives, pyrene cholesteryloleate, acridine, porphyrin, fluorescein, rhodamine, 16-diazorcortisone, ethidium, transition metal complexes of bleomycin, transition metal complexes of deglycobleomycin, organoplatinum complexes, alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavine adenine dinucleotide [FAD]), alloxazine mononucleotide (also known as flavine mononucleotide [FMN] and riboflavine-5-phosphate), vitamin Ks, vitamin L, their metabolites and precursors, and napththoquinones, naphthalenes, naphthols and their derivatives having planar molecular conformations, porphyrins, dyes such as neutral red, methylene blue, acridine, toluidines, flavine (acriflavine hydrochloride) and phenothiazine derivatives, coumarins, quinolones, quinones, and anthroquinones, aluminum (111) phthalocyanine tetrasulfonate, hematoporphyrin, and phthalocyanine, and compounds which preferentially adsorb to nucleic acids with little or no effect on proteins. The term “alloxazine” includes isoalloxazines.

Endogenously-based derivatives include synthetically derived analogs and homologs of endogenous photoactivated molecules, which may have or lack lower (1 to 5 carbons) alkyl or halogen substituents of the photosensitizers from which they are derived, and which preserve the function and substantial non-toxicity. Endogenous molecules are inherently non-toxic and may not yield toxic photoproducts after photoradiation.

Table 5 lists some photoactivatable molecules capable of being photoactivated to induce an auto vaccine effect.

TABLE 5

| | Excitation | Emission |
|---|---|---|
| Endogenous Fluorophores | Max. (nm) | Max. (nm) |
| Amino acids: | | |
| Tryptophan | 280 | 350 |
| Tyrosine | 275 | 300 |
| Phenylalanine | 260 | 280 |
| Structural Proteins: | | |
| Collagen | 325, 360 | 400, 405 |
| Elastin | 290, 325 | 340, 400 |
| Enzymes and Coenzymes: | | |
| flavin adenine dinucleotide | 450 | 535 |
| reduced nicotinamide dinucelotide | 290, 351 | 440, 460 |
| reduced nicotinamide dinucelotide | 336 | 464 |
| phosphate | | |
| Vitamins: | | |
| Vitamins A | 327 | 510 |
| Vitamins K | 335 | 480 |
| Vitamins D | 390 | 480 |
| Vitamins B₆ compounds: | | |
| Pyridoxine | 332, 340 | 400 |
| Pyridoxamine | 335 | 400 |
| Pyridoxal | 330 | 385 |
| Pyridoxic acid | 315 | 425 |
| Pyridoxal phosphate | 5′-330 | 400 |
| Vitamin B₁₂ | 275 | 305 |
| Lipids: | | |
| Phospholipids | 436 | 540, 560 |
| Lipofuscin | 340-395 | 540, 430-460 |
| Ceroid | 340-395 | 430-460, 540 |
| Porphyrins | 400-450 | 630, 690 |

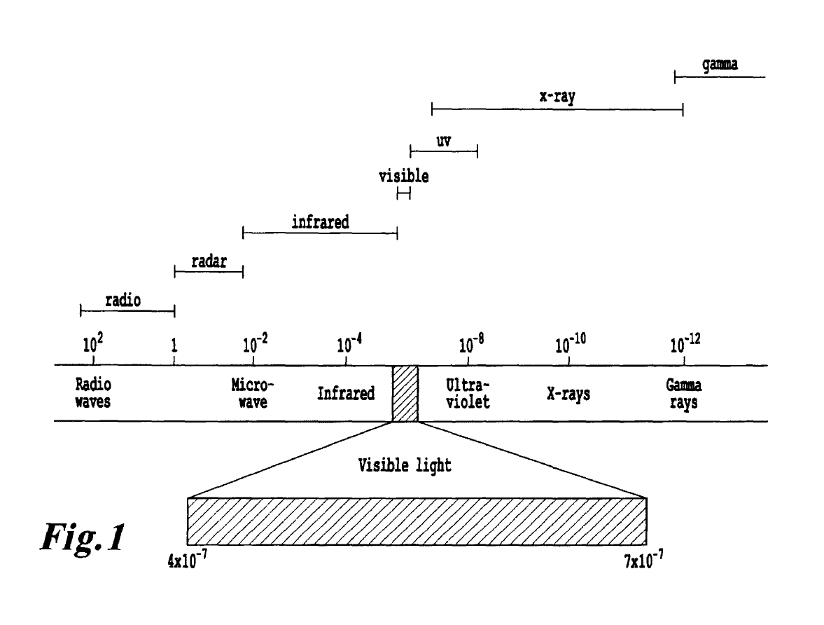

FIG. 1 provides an exemplary electromagnetic spectrum in meters (1 nm equals meters).

Although the activatable pharmaceutical agent e.g., a photoactive drug) and the energy modulation agent can be distinct and separate, it will be understood that the two agents need not be independent and separate entities. In fact, the two agents may be associated with each other via a number of different configurations. Where the two agents are independent and separately movable from each other, they generally interact with each other via diffusion and chance encounters within a common surrounding medium. Where the activatable pharmaceutical agent and the energy modulation agent are not separate, they may be combined into one single entity.

The initiation energy source can be any energy source capable of providing energy at a level sufficient to cause cellular changes directly or via a modulation agent which transfer the initiation energy to energy capable of causing the predetermined cellular changes. Also, the initiation energy source can be any energy source capable of providing energy at a level sufficient activate the activatable agent directly, or to provide the energy to a modulation agent with the input needed to emit the activation energy for the activatable agent (indirect activation). Preferable initiation energy sources include, but are not limited to, UV-A lamps or LEDs, UV-B lamps or LEDs, UV-C lamps or LEDs, or fiber optic lines, a light needle, an endoscope, and a linear accelerator that generates x-ray, gamma-ray, or electron beams.

In a preferred embodiment the initiation energy is capable of penetrating completely through the subject or target organ or target site being treated. Within the context of the invention, the phrase “capable of penetrating completely through the subject” is used to refer to energy that can penetrate to any depth within the subject to activate the activatable pharmaceutical agent. It is not required that the any of the energy applied actually pass completely through the subject, merely that it be capable of doing so in order to permit penetration to any desired depth to activate the activatable pharmaceutical agent. Exemplary initiation energy sources that are capable of penetrating completely through the subject include, but are not limited to, UV light, visible light, IR radiation, x-rays, gamma rays, electron beams, microwaves and radio waves. In one embodiment, the initiation energy can penetrate completely through the subject and can be applied from a single source or more than one source.

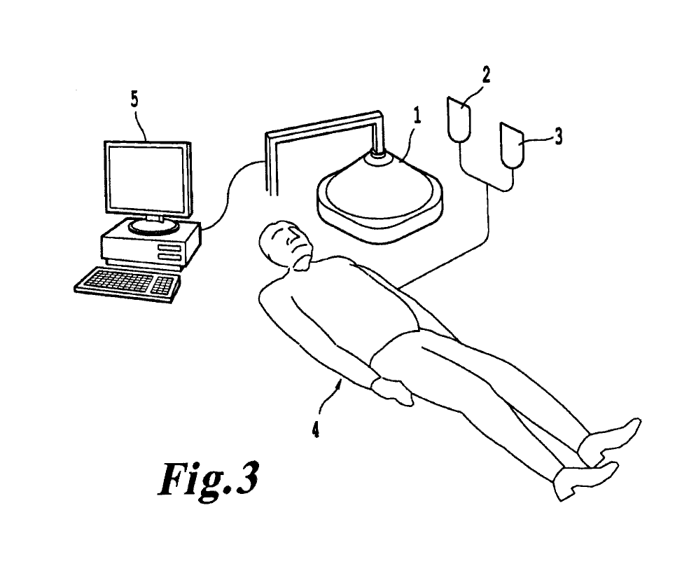

In another embodiment, the initiation energy source 1 (shown in FIG. 3) may be a linear accelerator equipped with image guided computer-control capability to deliver a precisely calibrated beam of radiation to a pre-selected coordinate. One example of such is the SmartBeam™ IMRT (intensity modulated radiation therapy) method from Varian medical methods (Varian Medical Methods, Inc., Palo Alto, Calif.). X-ray machines that produce from 10 to 150 keV X-rays are readily available in the marketplace. For instance, the General Electric Definium series or the Siemens MULTIX series are but two examples of typical X-ray machines designed for the medical industry, which could be used in the invention.

In one embodiment, the initiation energy may also be UV radiation, visible light, infrared radiation (IR), x-rays, gamma rays, an electron beam, microwaves or radio waves. Energy modulation agents (e.g., up converting or down converting agents) inside the subject emit the aforementioned light for selective activation into the subject.

An additional embodiment of the invention is to provide a method for treatment of a condition, disease or disorder by the in-situ generation of energy in a subject in need thereof, where the energy generated can be used directly to effect a change thereby treating the condition, disease or disorder, or the energy can be used to activate an activatable pharmaceutical agent, which upon activation effects a change thereby treating the condition, disease or disorder. The energy can be generated in-situ by any desired method, including, but not limited to conversion of an energy applied to the subject externally, which is converted in-situ to a different energy (of lower or higher energy than that applied), through the use of one or more energy modulation agents producing at least two different wavelengths of light, each wavelength of light associated with a different biological response. For example, light of first wavelength photoactivates a pharmaceutical agent (i.e., a photoactive or photoactivatable drug), and light of second wavelength heats the local treatment area.

A further embodiment of the invention combines the treatment of a condition, disease or disorder with the generation of heat in the affected target structure in order to enhance the effect of the treatment. For example, in the treatment of a cell proliferation disorder using a photoactivatable pharmaceutical agent (such as an alkylating agent, a psoralen or derivative thereof), one can activate the photoactivatable pharmaceutical agent by applying an initiation energy which, directly or indirectly, activates the pharmaceutical agent or agents by way of exposure of the pharmaceutical agent or agents to at least two different wavelengths of light, each wavelength of light associated with a different biological response. For example, light of first wavelength photactivates an alkylating agent, a psoralen or derivative thereof, and light of second wavelength heats the local treatment area.

As noted elsewhere in the present application, this initiation energy can be of any type, so long as it can be converted to an energy suitable for activating the pharmaceutical compound (i.e., the photoactive or photoactivatable drug). In addition to applying this initiation energy, in this embodiment of the invention, energy is applied that causes heating of the target structure. In the case of a cell proliferation disorder such as cancer, the heating would increase the proliferation rate of the cancer cells. While this may seem counterintuitive at first, when the cell proliferation disorder is being treated using a DNA intercalation agent, such as psoralen or a derivative thereof, this increase in cell proliferation can actually assist the psoralen in causing apoptosis. In particular, when psoralen becomes intercalated into DNA, apoptosis occurs when the cell goes through its next division cycle. By increasing the rate at which the cells divide, one can use the invention methods to enhance the onset of apoptosis.

Additional sources of heat can be utilized. Heat can be generated using the application of microwaves or NIR energy to the target structure or by the use of use of nanoparticles of metal or having metal shells. In the nanoparticles embodiment, as is done in tumor thermotherapy, magnetic metal nanoparticles can be targeted to cancer cells using conventional techniques, then used to generate heat by application of a magnetic field to the subject under controlled conditions. (DeNardo S J, DeNardo G L, Natarajan A et al.: Thermal dosimetry predictive of efficacy of 111In-ChL6 NPAMF-induced thermoablative therapy for human breast cancer in mice. J. Nucl. Med. 48(3), 437-444 (2007).)

Alternatively, one embodiment of the invention generates heat through the application of NIR to nanoparticles having metal shells which is converted into thermal energy. (Hirsch L R, Stafford R J, Bankson J et al.: Nanoshell-mediated near-infrared thermal therapy of tumors under magnetic resonance guidance. Proc. Natl Acad. Sci. USA100(23), 13549-13554 (2003)).

Photoactivatable agents may be in general stimulated by an energy source, such as UV or visible or infrared irradiation from the energy modulation agents at different wavelengths, resonance energy transfer, exciton migration, electron injection, or chemical reaction, to an activated energy state that is capable of effecting the predetermined cellular change desired. In a preferred embodiment, the photoactivatable agent, upon activation, binds to DNA or RNA or other structures in a cell. The activated energy state of the agent is capable of causing damage to cells, inducing apoptosis.

One preferred method of treating a condition, disorder or disease in a subject comprises:
(1) administering to the subject at least one activatable pharmaceutical agent that is capable of effecting a predetermined change to the target structure (e.g., a virus or bacterium) when activated; and
(2) applying an initiation energy from an initiation energy source to the subject,
   wherein the applied initiation energy activates the activatable agent (e.g., a photoactive or photoactivatable drug) in situ by different wavelengths of UV or visible or infrared irradiation emitted from at least one energy modulation agent in a vicinity of or within the target structure,
thus causing the predetermined change to the target structure to occur, wherein said predetermined change treats the condition, disorder, or disease (especially but not limited to viral or bacterial infections).

Another preferred method for treating a condition, disorder or disease in a subject, comprises:
(1) administering to the subject at least one activatable pharmaceutical agent (e.g., a photoactive or photoactivatable drug) that is capable of activation by a multi photon absorption event and of effecting a predetermined change in said target when activated by different wavelengths of UV or visible or infrared irradiation emitted from at least one energy modulation agent in a vicinity of or within the target structure; and
(2) applying an initiation energy from an initiation energy source to the subject,
   wherein the applied initiation energy activates the activatable agent (e.g., a photoactive or photoactivatable drug) by the multi photon absorption event in situ,
thus causing the predetermined change to occur, wherein said predetermined change treats the condition, disorder, or disease (especially but not limited to viral or bacterial infections.

The concept of multi-photon excitation is based on the idea that two or more photons of low energy can excite a fluorophore in a quantum event, resulting in the emission of a fluorescence photon, typically at a higher energy than the two or more excitatory photons.

Commonly used fluorophores have excitation spectra in the 400-500 nm range, whereas the laser used to excite the fluorophores lies in the ˜700-1000 nm (infrared) range. If the fluorophore absorbs two infrared photons simultaneously, it will absorb enough energy to be raised into the excited state. The fluorophore will then emit a single photon with a wavelength that depends on the type of fluorophore used (typically in the visible spectrum). Because two photons need to be absorbed to excite a fluorophore, the probability of emission is related to the intensity squared of the excitation beam. Therefore, a higher amount of two-photon fluorescence is generated where the laser beam is tightly focused than where it is more diffuse. Effectively, fluorescence is observed in any appreciable amount in the focal volume, resulting in a high degree of rejection of out-of-focus objects.

In one embodiment, the energy upconversion is obtained via 2, 3, 4, or 5 simultaneous photon absorptions.

Yet another preferred method for treating a condition, diseases, or disorder in a subject, comprises:
(1) administering to the subject at least one energy modulation agent and at least one activatable pharmaceutical agent (e.g., a photoactive or photoactivatable drug) that is capable effecting a predetermined cellular change when activated by different wavelengths of UV or visible or infrared irradiation emitted from the at least one energy modulation agent in a vicinity of or within the target structure; and
(2) applying an initiation energy from an initiation energy source to the subject,
wherein the energy modulation agent upgrades the applied initiation energy to an energy, which then activates the activatable agent in situ,
thus causing the predetermined cellular change to occur, wherein said predetermined cellular change treats the condition, disorder, or disease (preferably but not limited to viral or bacterial infections.

In yet another aspect, the radiative energy may be of a higher energy than the excitation energy of the photoactive or photoactivatable agent. In this aspect, the photoactive agent may be activated via an “energy downgrade” mechanism (down conversion to lower energy). In one scenario, via the multi-photon mechanism, two lower energy photons having energy x may be absorbed by an agent to excite the agent from ground state E0 to a higher energy state E2. The agent may then relax down to an intermediate energy state E1 by emitting a photon having an energy y that is equal to the energy gap between E2 and E1, where y is less than x. Other mechanisms of energy downgrade may be mediated by energy transformation agents such as quantum dots, nanotubes, or other phosphorescent or fluorescent agents having suitable photo-radiation properties. Thus, yet another preferred method for treating a condition, disease, or disorder in a subject, comprises:
(1) administering to the subject at least one energy modulation agent and at least one activatable pharmaceutical agent (e.g., a photoactive or photoactivatable drug) that is capable of activation by multi photon absorption and of effecting a predetermined cellular change when activated by different wavelengths of UV or visible or infrared irradiation emitted from the at least one energy modulation agent in the subject; and
(2) applying an initiation energy from an initiation energy source to the subject,
wherein the energy modulation agent downgrades the applied initiation energy to an energy, which then activates the activatable agent by a multi photon absorption event in situ,
   thus causing the predetermined cellular change to occur, wherein said predetermined cellular change treats the condition, disease, or disorder (preferably but not limited to viral or bacterial infections).

Thus, yet another preferred method for treating a condition, disease, or disorder in a subject, comprises:
(1) administering to the subject at least one energy modulation agent and at least one activatable pharmaceutical agent (e.g., a photoactive or photoactivatable drug) that is capable of effecting a predetermined cellular change when activated by different wavelengths of UV or visible or infrared irradiation emitted from the at least one energy modulation agent in the subject; and
(2) applying an initiation energy from an initiation energy source to the subject,
wherein the energy modulation agent downgrades the applied initiation energy to an energy, which then activates the activatable agent in situ,
   thus causing the predetermined cellular change to occur, wherein said predetermined cellular change treats the condition, disorder or disease (preferably but not limited to viral or bacterial infections).

In a further preferred embodiment, the invention provides a method for treating a condition, disorder or disease in a subject, comprising:

(1) administering to the subject an activatable pharmaceutical agent (e.g., a photoactive or photoactivatable drug) that is capable of effecting a predetermined change in said target structure when activated by different wavelengths of UV or visible or infrared irradiation emitted from at least one energy modulation agent in the subject; and

(2) applying an initiation energy from an initiation energy source to the subject,

wherein the initiation energy applied and activatable pharmaceutical agent upon activation produce a controlled amount of singlet oxygen in the subject to produce cell lysis, and wherein the initiation energy activates the activatable pharmaceutical agent in situ,
thus causing the predetermined change to occur to the subject, wherein said predetermined change targets the condition, disorder or disease (preferably but not limited to viral or bacterial infections).

In a different preferred embodiment, the invention provides a method for treating a condition, disorder or disease in a subject, comprising:

(1) administering to the subject an activatable pharmaceutical agent (e.g., a photoactive or photoactivatable drug) that is capable of activation by multi photon absorption and effecting a predetermined change in the subject when activated by different wavelengths of UV or visible or infrared irradiation emitted from at least one energy modulation agent in the subject; and

(2) applying an initiation energy from an initiation energy source to the subject,

wherein the initiation energy applied and activatable pharmaceutical agent upon activation produce insufficient singlet oxygen in the subject to produce cell lysis, and wherein the initiation energy activates the activatable pharmaceutical agent by the multi photon absorption event in situ,
thus causing the predetermined change to occur, wherein said predetermined change targets the condition, disorder or disease.

In a different preferred embodiment, the invention provides a method for treating a condition, disorder or disease in a subject, comprising:

(1) administering to the subject an activatable pharmaceutical agent (e.g., a photoactive or photoactivatable drug) that is capable of activation by multi photon absorption and effecting a predetermined change in said target structure when activated by different wavelengths of UV or visible or infrared irradiation emitted from at least one energy modulation agent in the subject; and

(2) applying an initiation energy from an initiation energy source to the subject,

wherein the initiation energy applied and activatable pharmaceutical agent upon activation produce a controlled amount of singlet oxygen in the subject to produce cell lysis, and wherein the initiation energy activates the activatable pharmaceutical agent by the multi photon absorption event in situ,
thus causing the predetermined change to occur, wherein said predetermined change targets the condition, disorder or disease (preferably but not limited to viral or bacterial infections).

Work in the area of photodynamic therapy has shown that the amount of singlet oxygen required to cause cell lysis, and thus cell death, is 0.32×10⁻³ mol/liter or more, or 10⁹ singlet oxygen molecules/cell or more. In one embodiment, it is preferable to avoid production of an amount of singlet oxygen that would cause cell lysis, due to its indiscriminate nature of attack, lysing both target cells and healthy cells. Accordingly, in one embodiment, the level of singlet oxygen production caused by the initiation energy used or activatable pharmaceutical agent upon activation is less than level needed to cause cell lysis.

One advantage is that multiple wavelengths of emitted radiation may be used to selectively stimulate one or more photoactivatable agents or energy modulation agents capable of stimulating the one or more photoactivatable agents. The energy modulation agent is preferably stimulated at a wavelength and energy that causes little or no damage to healthy cells, with the energy from one or more energy modulation agents being transferred, such as by Foerster Resonance Energy Transfer, to the photoactivatable agents that damage the cell and cause the onset of the desired cellular change, e.g., apoptosis of the cells.

Another advantage is that side effects can be greatly reduced by limiting the production of free radicals, singlet oxygen, hydroxides and other highly reactive groups that are known to damage healthy cells. Furthermore, additional additives, such as antioxidants, may be used to further reduce undesired effects of irradiation.

In a different embodiment, it is preferable to control the amount of singlet oxygen that would cause cell lysis relative to the amount of activated psoralen or alkylating agent produced.

Accordingly, in this embodiment, the level of singlet oxygen production caused by the initiation energy used or activatable pharmaceutical agent upon activation is less than or equal to the amount of activated psoralen or alkylating agent. For example, the amount of singlet oxygen produced can range from 1% to 95% of the activated psoralen produced. In another example, the amount of singlet oxygen produced can range from 10% to 90% of the activated psoralen or alkylating agent produced. In another example, the amount of singlet oxygen produced can range from 20% to 80% of the activated psorlen produced. In another example, the amount of singlet oxygen produced can range from 30% to 70% of the activated psoralen or alkylating agent produced. In another example, the amount of singlet oxygen produced can range from 40% to 60% of the activated psoralen or alkylating agent produced.

In a different embodiment, it is preferable to control the amount of singlet oxygen to be more than or equal to the amount of activated psoralen or alkylating agent. For example, the amount of activated psoralen or alkylating agent produced can range from 1% to 95% of the singlet oxygen produced. In another example, the amount of activated psoralen or alkylating agent produced can range from 10% to 90% of the singlet oxygen produced. In another example, the amount of activated psoralen or alkylating agent produced can range from 20% to 80% of the singlet oxygen produced. In another example, the amount of activated psoralen or alkylating agent n produced can range from 30% to 70% of t or alkylating agent the singlet oxygen produced. In another example, the amount of activated psoralen or alkylating agent produced can range from 40% to 60% of the singlet oxygen produced.

Resonance Energy Transfer (RET) is an energy transfer mechanism between two molecules having overlapping emission and absorption bands. Electromagnetic emitters are capable of converting an arriving wavelength to a longer wavelength. For example, UV-B energy absorbed by a first molecule may be transferred by a dipole-dipole interaction to a UV-A-emitting molecule in close proximity to the UV-B-absorbing molecule. Alternatively, a material absorbing a shorter wavelength may be chosen to provide RET to a non-emitting molecule that has an overlapping absorption band with the transferring molecule's emission band. Alternatively, phosphorescence, chemiluminescence, or bioluminescence may be used to transfer energy to a photoactivatable molecule.

Yet another example is that nanoparticles or nanoclusters of certain atoms may be introduced such that are capable of resonance energy transfer over comparatively large distances, such as greater than one nanometer, more preferably greater than five nanometers, even more preferably at least 10 nanometers. Functionally, resonance energy transfer may have a large enough “Foerster” distance (R₀), such that nanoparticles in one part of a cell are capable of stimulating activation of photoactivatable agents disposed in a distant portion of the cell, so long as the distance does not greatly exceed R₀. For example, gold nanospheres having a size of 5 atoms of gold have been shown to have an emission band in the ultraviolet range, recently.

In one embodiment, an aggressive cell proliferation disorder has a much higher rate of mitosis, which leads to selective destruction of a disproportionate share of the malignant cells during even a systemically administered treatment. Stem cells and healthy cells may be spared from wholesale programmed cell death, even if exposed to photoactivated agents, provided that such photoactivated agents degenerate from the excited state to a lower energy state prior to binding, mitosis or other mechanisms for creating damage to the cells of a substantial fraction of the healthy stem cells. Thus, in this embodiment, an auto-immune response may not be induced.

Alternatively, a blocking agent may be used that prevents or reduces damage to stem cells or healthy cells, selectively, which would otherwise be impaired. The blocking agent is selected or is administered such that the blocking agent does not impart a similar benefit to malignant cells, for example.

Any of the photoactivatable agents described herein may be exposed to an excitation energy source implanted in a subject preferably near a target site. The photoactive or photoactivatable agent may be directed to a receptor site by a carrier having a strong affinity for the receptor site. Within the context of the invention, a “strong affinity” is preferably an affinity having an equilibrium dissociation constant, Kᵢ, at least in the nanomolar, nM, range or higher. Preferably, the carrier may be a polypeptide and may form a covalent bond with a photoactive or photoactivatable agent, for example. The polypeptide may be an insulin, interleukin, thymopoietin or transferrin, for example. Alternatively, a photoactive or photoactivatable agent may have a strong affinity for the target cell without binding to a carrier.

A receptor site may be any of the following: nucleic acids of nucleated blood cells, molecule receptor sites of nucleated blood cells, the antigenic sites on nucleated blood cells, epitopes, or other sites where photoactive or photoactivatable agents are capable of destroying a targeted cell.

In one embodiment, thin fiber optic lines are inserted in the subject and external light is used to photoactivate the agents. In another embodiment, a plurality of sources for supplying electromagnetic radiation energy or energy transfer can be used.

The phenomenon of ultra-weak emission from cellular systems has been a topic of various inquiries since the 1900s. This topic can be traced back to the early investigations of the Russian biologist Gurwitsch Alexander G. Gurwitsch more than seventy years ago, who speculated that ultraweak photon emission transmit information in cells [A. G. Gurwitsch, S. S. Grabje, and S. Salkind, “Die Natur des spezifischen Erregers der Zellteilung,” Arch. Entwicklungsmech. Org. 100, 11-40, 1923].

In the 1970s, this area of research was investigated by a number of investigators. The presence of biological radiation from a variety of cells was later investigated by several research groups in Europe and Japan using low-noise, sensitive photon-counting detection systems [B. Ruth and F.-A. Popp, “Experimentelle Untersuchungen zur ultraschwachen Photonenemission biologischer Systeme,” Z. Naturforsch., A: Phys. Sci. 31c, 741-745, 1976; T. I. Quickenden and S. S. Que-Hee, “The spectral distribution of the luminescence emitted during growth of the yeast Saccharomyces cerevisiae and its relationship to mitogenetic radiation,” Photochem. Photobiol. 23, 201-204, 1976; H. Inaba, Y. Shimizu, Y. Tsuji, and A. Yamagishi, “Photon counting spectral analysing system of extra-weak chemi- and bioluminescence for biochemical applications,” Pholochem. Photobiol. 30, 169-175, 1979]. Popp and coworkers suggested the evidence of some ‘informational character’ associated with the ultra-weak photon emission from biological systems, often referred by Popp as “bio-photons.” Other studies reported ultra-weak photon emission from various species including plant, and animals cells [H. J. Niggli, C. Scaletta, Y. Yan, F.-A. Popp, and L. A. Applegate, “Ultraweak photon emission in assessing bone growth factor efficiency using fibroblastic differentiation,” J. Photochem. Photobiol., B, 64, 62-68, 2001;]. Results of experiments of UV-irradiated skin fibroblasts indicated that repair deficient xeroderma pigmentosum cells show an efficient increase of ultraweak photon emission in contrast to normal cells. [H. J. Niggli, “Artificial sunlight irradiation induces ultraweak photon emission in human skin fibroblasts,” J. Photochem. Photobiol., B 18, 281-285 (1993)].

A delayed luminescence emission was also observed in biological systems [F.-A. Popp and Y. Yan, “Delayed luminescence of biological systems in terms of coherent states,” Phys. Lett. A 293, 93-97 (2002); A. Scordino, A. Triglia, F. Musumeci, F. Grasso, and Z. Rajfur, “Influence of the presence of Atrazine in water on in-vivo delayed luminescence of acetabularium acetabulum,” J. Photochem. Photobiol., B, 32, 11-17 (19%); This delayed luminescence was used in quality control of vegetable products [A. Triglia, G. La Malfa, F. Musumeci, C. Leonardi, and A. Scordino, “Delayed luminescence as an indicator of tomato fruit quality,” J. Food. Sci. 63, 512-515 (1998)] or for assessing the quality or quality changes of biological tissues [Yu Yan, Fritz-Albert Popp *, Sibylle Sigrist, Daniel Schlesinger, Andreas Dolf, Zhongchen Yan, Sophie Cohen, Amodsen Chotia, “Further analysis of delayed luminescence of plants”, Journal of Photochemistry and Photobiology B: Biology 78, 235-244 (2005)].

It has been reported that UV excitation can further enhance the ultra-weak emission and a method for detecting UV-A-laser-induced ultra-weak photon emission was used to evaluate differences between cancer and normal cells. [H. J. Niggli et al, Laser-ultraviolet-A-induced ultraweak photon emission in mammalian cells, Journal of Biomedical Optics 10(2), 024006 (2005)].

Accordingly, in one embodiment of the invention, upon applying an initiation energy from at least one source to a subject in need of treatment, the initiation energy induces a predetermined change in said target structure in situ by exposure of the target structure to different wavelengths of UV or visible or infrared irradiation emitted from at least one energy modulation agent in the subject,

wherein the predetermined change is the enhancement of energy emission, which then mediates, initiates or enhances a biological activity of the subject, or of a second type of target structure (e.g., a different cell type). Here, a first wavelength would induce the predetermined change (e.g., a treatment of a viral or bacterial infection) and a second wavelength would mediates, initiates or enhances a biological activity of other target structures in the subject (e.g., a different cell type).

In a further embodiment, a biocompatible emitting source, such as a fluorescing metal nanoparticle or fluorescing dye molecule, is selected that emits in the UV-A band. The UV-A emitting source is directed to the site of a disease or viral or bacterial condition. The UV-A emitting source may be directed to the site of the disease or viral or bacterial condition by systemically administering the UV-A emitting source. Preferably, the UV-A emitting source is concentrated in the target site, such as by physical insertion or by conjugating the UV-A emitting molecule with a specific carrier that is capable of concentrating the UV-A emitting source in a specific target structure, as is known in the art.

In one embodiment, the UV-A emitting source is a gold nanoparticle comprising a cluster of 5 gold atoms, such as a water soluble quantum dot encapsulated by polyamidoamine dendrimers. The gold atom clusters may be produced (for example according to procedures known in the art) through a slow reduction of gold salts (e.g. HAuCl₄ or AuBr₃) or other encapsulating amines, for example. One advantage of such a gold nanoparticle is the increased Foerster distance (i.e. R₀), which may be greater than 100 angstroms.

In one embodiment of this invention, in addition to the UV-A emitting source, a UV-B or UV-C emitting source is directed to the site of a disease or viral or bacterial condition to act as a germicide. In one embodiment of this invention, in addition to the UV-A emitting source, a NIR emitting source is directed to the site of a disease or condition to act as an anti-inflammatory or to promote cellular proliferation or to reduce pain. A number of commercially available drugs described below could also be activated by the NIR emitting source.

Porfimer sodium (Photofrin; QLT Therapeutics, Vancouver, BC, Canada), is a partially purified preparation of hematoporphyrin derivative (HpD). Photofrin has been approved by the US Food and Drug Administration for the treatment of obstructing esophageal cancer, microinvasive endobronchial non-small cell lung cancer, and obstructing endobronchial non-small cell lung cancer. Photofrin is activated with 630 nm, which has a tissue penetration of approximately 2 to 5 mm. Photofrin has a relatively long duration of skin photosensitivity (approximately 4 to 6 weeks).

Tetra (m-hydroxyphenyl) chlorin (Foscan; Scotia Pharmaceuticals, Stirling, UK), is a synthetic chlorine compound that is activated by 652 nm light. Clinical studies have demonstrated a tissue effect of up to 10 mm with Foscan and 652 nm light. Foscan is more selectively a photosensitizer in tumors than normal tissues, and requires a comparatively short light activation time. A recommended dose of 0.1 mg/kg is comparatively low and comparatively low doses of light may be used. Nevertheless, duration of skin photosensitivity is reasonable (approximately 2 weeks). However, Foscan induces a comparatively high yield of singlet oxygen, which may be the primary mechanism of DNA damage for this molecule.

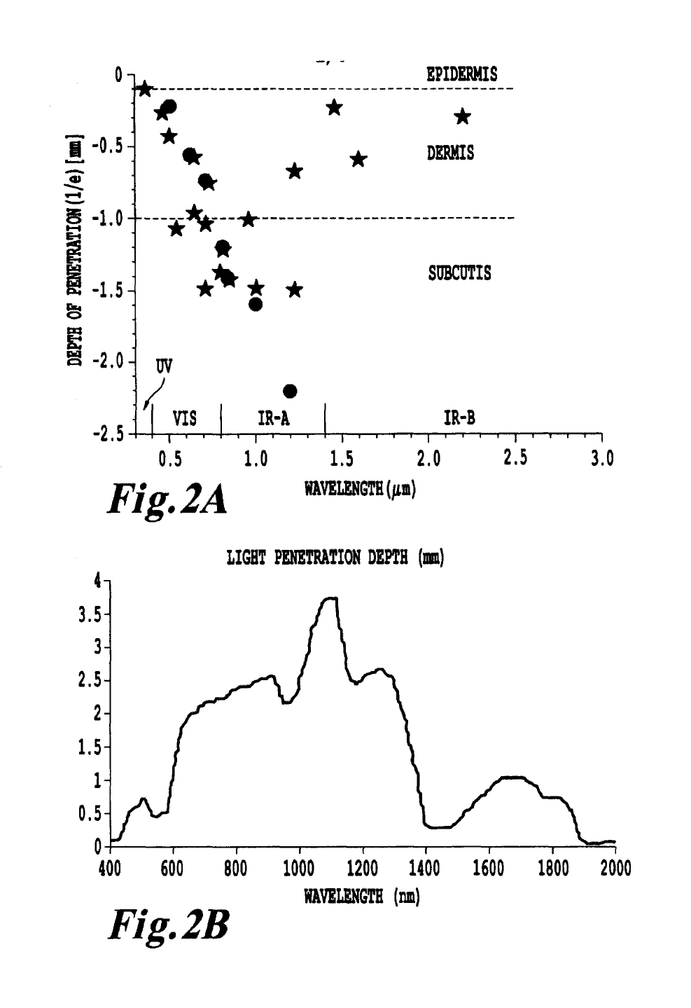

Motexafin lutetium (Lutetium texaphryin) is activated by light in the near infared region (732 nm). Absorption at this wavelength has the advantage of potentially deeper penetration into tissues, compared with the amount of light used to activate other photosensitizers (FIGS. 2A and 2B). Lutetium texaphryin also has one of the greatest reported selectivities for tumors compared to selectivities of normal tissues. Young S W, et al.: Lutetium texaphyrin (PCI-0123) a near-infrared, water-soluble photosensitizer. Photochem Photobiol 1996, 63:892-897. In addition, its clinical use is associated with a shorter duration of skin photosensitivity (24 to 48 hours). Lutetium texaphryin has been evaluated for metastatic skin cancers. It is currently under investigation for treatment of recurrent breast cancer and for locally recurrent prostate cancer. The high selectivity for tumors promises improved results in clinical trials.

While the description of the invention describes specific examples using nanoparticles, the invention in many embodiments is not limited to particles having a size less than one micron. However, in many of the embodiments, the size range of having a size less than one micron, and especially less than 100 nm produces properties of special interest such as for example emission lifetime luminescence quenching, luminescent quantum efficiency, and concentration quenching and such as for example diffusion, penetration, and dispersion into mediums where larger size particles would not migrate.

In one embodiment, similar to that described above, the first wavelength would induce the predetermined change (e.g., treatment of viral or bacterial infection) and the second wavelength would mediates, initiates or enhances neuronal spike firing

For example, a light-sensitive protein (for example, channelrhodopsin-2 (ChR2) and chloride pump halorhodopsin (NpHR)) can be incorporated into the lentiviral vector or other vector providing delivery of the light-sensitive protein encoding gene into a target cell. ChR2 containing a light sensor and a cation channel, provides electrical stimulation of appropriate speed and magnitude to activate neuronal spike firing, when the cells harboring Ch2R are pulsed with light.

In one embodiment for use as either the first or second wavelength, a lanthanide chelate capable of intense luminescence is used. For example, a lanthanide chelator may be covalently joined to a coumarin or coumarin derivative or a quinolone or quinolone-derivative sensitizer. Sensitizers may be a 2- or 4-quinolone, a 2- or 4-coumarin, or derivatives or combinations of these examples. A carbostyril 124 (7-amino-4-methyl-2-quinolone), a coumarin 120 (7-amino-4-methyl-2-coumarin), a coumarin 124 (7-amino-4-(trifluoromethyl)-2-coumarin), aminoinethyltrimethylpsoralen or other similar sensitizer may be used. Chelates may be selected to form high affinity complexes with lanthanides, such as terbium or europium, through chelator groups, such as DTPA. Such chelates may be coupled to any of a wide variety of well-known probes or carriers, and may be used for resonance energy transfer to a psoralen or psoralen-derivative, such as 8-MOP, or other photoactive or photoactivatable molecules capable of binding DNA. In one alternative example, the lanthanide chelate is localized at the site of the disease or viral or bacterial infection using an appropriate carrier molecule, particle or polymer, and a source of electromagnetic energy is introduced by minimally invasive procedures to irradiate the target structure, after exposure to the lanthanide chelate and a photoactive or photoactivatable molecule.

In another embodiment for use as either the first or second wavelength, a biocompatible, endogenous fluorophore emitter is selected to stimulate resonance energy transfer to a photoactivatable molecule. A biocompatible emitter with an emission maxima within the absorption range of the biocompatible, endogenous fluorophore emitter may be selected to stimulate an excited state in fluorophore emitter. One or more halogen atoms may be added to any cyclic ring structure capable of intercalation between the stacked nucleotide bases in a nucleic acid (either DNA or RNA) to confer new photoactive properties to the intercalator. Any intercalating molecule (psoralens, coumarins, or other polycyclic ring structures) may be selectively modified by halogenation or addition of non-hydrogen bonding ionic substituents to impart advantages in its reaction photochemistry and its competitive binding affinity for nucleic acids over cell membranes or charged proteins, as is known in the art.

In general, any source for activation of the pharmaceutical agent, such as electrical, chemical and/or radiation, can be used individually or combined into a system for activating an activatable molecule. The process may be a photopheresis process or may be similar to photophoresis. While photophoresis is generally thought to be limited to photonic excitation, such as by UV-light, other forms of radiation may be used as a part of a system to activate an activatable molecule. Radiation includes ionizing radiation which is high energy radiation, such as an X-ray or a gamma ray, which interacts to produce ion pairs in matter. Radiation also includes high linear energy transfer irradiation, low linear energy transfer irradiation, alpha rays, beta rays, neutron beams, accelerated electron beams, and ultraviolet rays. Radiation also includes proton, photon and fission-spectrum neutrons. Higher energy ionizing radiation may be combined with chemical processes to produce energy states favorable for resonance energy transfer, for example. Other combinations and variations of these sources of excitation energy may be combined as is known in the art, in order to stimulate the activation of an activatable molecule, such as 8-MOP or various alkylating agents. In one example, ionizing radiation is directed at a solid tumor and stimulates, directly or indirectly, activation of 8-MOP, as well as directly damaging the DNA of malignant tumor cells. In this example, either the effect of ionizing radiation or the photophoresis-like activation of 8-MOP or 1,5-iodonophthylazide may be thought of as an adjuvant therapy to the other.

In one embodiment, the invention provides a method for treating a condition, disease or disorder (e.g., a viral or bacterial infection) in a subject, comprising:
(1) administering to the subject an activatable pharmaceutical agent (e.g., photoactive or photoactivatable drug) that is capable of effecting a predetermined change by exposure of the subject to different wavelengths of UV or visible or infrared irradiation emitted from at least one energy modulation agent in a target region of the subject; and
(2) applying an initiation energy from an initiation energy source to the subject,
   wherein the initiation energy source is a source of energy capable of penetrating completely through the subject, and wherein the applying activates the activatable agent in situ,
thus causing the predetermined change to occur, wherein occurrence of the predetermined change causes an increase in rate or decrease in rate of cell division and/or growth to treat the condition, disease or disorder (e.g., viral or bacterial infection).

In a further embodiment, the invention provides a method for treating a condition, disease or disorder (e.g., a viral or bacterial infection) in a subject, comprising:

(1) administering to the subject one or more energy modulation agents and an activatable pharmaceutical agent that is capable of effecting a predetermined change in the subject when activated by exposure of the target structure to different wavelengths of UV or visible or infrared irradiation emitted from at least one energy modulation agent in a target region of subject; and

(2) applying an initiation energy from an initiation energy source to the subject,

wherein the one or more energy modulation agents convert the initiation energy applied to UV-A or visible energy, which then activates the activatable agent in situ,
thus causing the predetermined change to occur, wherein occurrence of the predetermined change causes an increase in rate or decrease in rate of cell division and/or growth to treat the condition, disease or disorder (preferably but not limited to viral or bacterial infections).

In a different embodiment, the activatable pharmaceutical agent can be activated by a single or multiphoton absorption event.

In yet another embodiment, the activatable pharmaceutical agent, preferably a photoactive or photoactivatable agent, is directed to a receptor site by a carrier having a strong affinity for the receptor site. The carrier may be a polypeptide and may form a covalent bond with a photo active agent, for example. The polypeptide may be an insulin, interleukin, thymopoietin or transferrin, for example. Alternatively, a photoactive or photoactivatable pharmaceutical agent may have a strong affinity for the target cell without a binding to a carrier.

For example, a treatment may be applied that acts to slow or pause mitosis. Such a treatment is capable of slowing the division of rapidly dividing healthy cells or stem cells without pausing mitosis of cancerous cells. Thus, the difference in growth rate between the non-target cells and target cells are further differentiated to enhance the effectiveness of the methods of the invention.

In a further embodiment, methods in accordance with the invention may further include adding an additive to alleviate treatment side-effects. Exemplary additives may include, but are not limited to, antioxidants, adjuvant, or combinations thereof. In one exemplary embodiment, psoralen is used as the activatable pharmaceutical agent, UV-A is used as the activating energy, and antioxidants are added to reduce the unwanted side-effects of irradiation.

In another aspect, the invention also provides methods for producing an autovaccine, including: (1) providing a population of targeted cells; (2) treating the cells ex vivo with a psoralen or a derivative thereof or an alkylating agent; (3) activating the psoralen or alkylating agent with an initiation energy source to induce a predetermined change in a target structure in the population of the target cells by exposure of the target structure to at least one wavelength of UV or visible or infrared irradiation emitted from at least one energy modulation agent in a vicinity of or within the target structure; and (4) returning the treated cells back to the host to induce an autovaccine effect against the targeted cell, wherein the treated cells cause an autovaccine effect.

In a different embodiment, a method for generating an autovaccine for a subject, comprises:
(1) providing a population of target cells;
(2) treating the target cells ex vivo in an environment separate and isolated from the subject with an activatable pharmaceutical agent (e.g., a photoactive or photoactivatable drug) capable of activation by a multi photon absorption event;
(3) exposing the treated target cells to an energy source;
(4) activating the activatable pharmaceutical agent with the energy source by the multi photon absorption event to induce a predetermined change in at least one target structure in the target cells by exposure of the target cells to different wavelengths of UV or visible or infrared irradiation emitted from at least one energy modulation agent in a vicinity of or within the target cell; and
(5) returning the thus changed cells back to the subject to induce in the subject an autovaccine effect against the target cells.

In a further embodiment, methods in accordance with the invention may further include a method for modifying a target structure which mediates or is associated with a biological activity, comprising:
(1) contacting said target structure with at least one activatable pharmaceutical agent (e.g., a photoactive or photoactivatable drug) that is capable of effecting a predetermined change in a target structure when activated and at least one plasmonics-active agent; and
(2) applying an initiation energy from an initiation energy source to target structure to expose the target structure to different wavelengths of UV or visible or infrared irradiation emitted from at least one energy modulation agent in a vicinity of or within the target structure,
   wherein the plasmonics-active agent enhances or modulates the applied initiation energy, such that the enhanced initiation energy activates the activatable agent
thus causing the predetermined change to the target structure to occur, wherein said predetermined change modifies the target structure and modulates the biological activity of the target structure.

In a different embodiment, the predetermined change enhances the expression of, promotes the growth of, or increases the quantity of the target structure; enhances, inhibits or stabilizes the usual biological activity of said target structure compared to a similar untreated target structure, and/or alters the immunological or chemical properties of said target structure. In a different embodiment, the target structure is a compound that is modified by said predetermined change to be more or less antigenic or immunogenic

The activatable pharmaceutical agent (e.g., the photoactive or photoactivatable agent) and derivatives thereof as well as the energy modulation agent, can be incorporated into pharmaceutical compositions suitable for administration. Such compositions typically comprise the activatable pharmaceutical agent and a pharmaceutically acceptable carrier. The pharmaceutical composition also comprises at least one additive having a complementary therapeutic or diagnostic effect, wherein the additive is one selected from an antioxidant, an adjuvant, or a combination thereof.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Modifications can be made to the compound of the invention to affect solubility or clearance of the compound. These molecules may also be synthesized with D-amino acids to increase resistance to enzymatic degradation. If necessary, the activatable pharmaceutical agent can be co-administered with a solubilizing agent, such as cyclodextran.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, rectal administration, and direct injection into the affected area, such as direct injection into a tumor. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition should be sterile and should be fluid to the extent that easy syringability exists. It should be stable under the conditions of manufacture and storage and should be preserved against the contaminating action of microorganisms such as bacteria and fungi.

The carrier of the energy modulation agent and/or the photoactivatable agent can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds (i.e., the energy modulation agents and photoactivatable drugs noted above) are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 (the entire contents of which are incorporated herein by reference).

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions containing the photoactivatable agents and/or the energy modulation agents can be included in a container, pack, or dispenser together with instructions for administration.

In one embodiment of the invention, there is provided a pharmaceutical composition for modifying a target structure. The pharmaceutical composition includes at least one agent selected from the group consisting of energy modulation agents, photoactivatable drugs, plasmonics-active agents and combinations thereof. The energy modulation agents include one or more light emitters capable of emitting at least one or more wavelengths of light, each wavelength of light associated with a biological response. The pharmaceutical composition preferably includes a pharmaceutically acceptable carrier.

Methods of administering the various agents of this invention according to the invention are not limited to the conventional means such as injection or oral infusion, but include more advanced and complex forms of energy transfer. For example, genetically engineered cells that carry and express energy modulation agents may be used. Cells from the host may be transfected with genetically engineered vectors that express bioluminescent agents. Transfection may be accomplished via in situ gene therapy techniques such as injection of viral vectors or gene guns, or may be performed c; vivo by removing a sample of the host's cells and then returning to the host upon successful transfection.

Such transfected cells may be inserted or otherwise targeted at the site where diseased cells are located. In this embodiment, the initiation energy source may be a biochemical source as such ATP, in which case the initiation energy source is considered to be directly implanted in the transfected cell. Alternatively, a conventional micro-emitter device capable of acting as an initiation energy source may be transplanted at the site of the diseased cells.

In another embodiment of the invention, a booster treatment is utilized. A “booster treatment” in one embodiment could involve re-injecting one or more photoactivatable drugs and radiating the patient again. A “booster treatment” in another embodiment could involve re-injecting the subject one or more photoactivatable drugs and an energy modulation agent (e.g., a phosphor) and radiating the patient again. A “booster treatment” in another embodiment could involve radiating the subject again, but at a radiation level considered to be at either a palliative or therapeutic level. The purpose of these “booster” treatments is to activate the immune response initially or originally generated within the patient during the initial treatments.

In one embodiment of the booster treatment, the phosphor concentration can be increased 2-10 times, and the amount of photoactivatable drug can be increased 2-4 times, and the treatment frequency can increased to for example five (5) treatments in five (5) consecutive days. Furthermore, the timing between the prime (initial treatment sessions) and the booster treatment can be set to allow for an initial cellular immune response, followed by a period of homeostasis, most typically weeks or months after the initial priming treatment.

The invention can utilize one or more booster treatments in a manner similar to that described by David L. Woodland in their paper in TRENDS in Immunology Vol. 25 No. 2 Feb. 2004, entitled “Jump-Starting the Immune System: Prime-Boosting Comes of Age” (the entire contents of which are incorporated herein by reference). The basic prime-boost strategy involves priming the immune system to a target antigen, or a plurality of antigens created by the drug and/or radiation induced cell kill, and then selectively boosting this immunity by re-exposing the antigen or plurality of antigens in the boost treatment. One key strength of this strategy in the present invention is that greater levels of immunity are established by heterologous prime-boost than can be attained by a single vaccine administration or homologous boost strategies. For example, the initial priming events elicited by a first exposure to an antigen or a plurality of antigens appear to be imprinted on the immune system.

Here, in this invention and without limitation as to the details but rather for the purpose of explanation, the initial treatment protocol develops antibodies or cellular immune responses during the initial treatment. These “initial” responses can then be stimulated by the occurrence of a large number of newly created drug-modified or X-ray modified cells. As such, the patient's immune system would mount a more robust response against the disease than would be realized in a single treatment series.

In one embodiment of the invention, prior to the initial treatment or prior to booster treatments, the immune system of the subject could be further stimulated by injection of a more conventional vaccine such as for example a tetanus vaccine. Prior work by others has shown the efficacy of a tetanus booster to bolster the immune system's attack on a tumor by helping cancer vaccines present in the subject migrate to the lymph nodes, activating an immune response. Here, in this invention, the autovaccines generated internally from the treatments described above could also benefit from this effect.

It will also be understood that the order of administering the different agents is not particularly limited. Thus in some embodiments the activatable pharmaceutical agent may be administered before the energy modulation agent, while in other embodiments the energy modulation agent may be administered prior to the activatable pharmaceutical agent. It will be appreciated that different combinations of ordering may be advantageously employed depending on factors such as the absorption rate of the agents, the localization and molecular trafficking properties of the agents, and other pharmacokinetics or pharmacodynamics considerations.

A further embodiment is the use of the invention for the treatment of a viral or bacterial infection. In this example, a photoactivatable agent, preferably psoralen or an alkylating agent, is given to the patient, and is present in the blood supply. An activation source having limited penetration ability (such as UV or IR) is shined directly on through the skin or external covering such as the mucosa of the tongue. With the use of an IR source, the irradiation would penetrate deeper and generate UV via two single photon events with psoralen. The subcutaneous blood supply is exposed to one or more wavelengths of UV or visible or infrared irradiation emitted from at least one energy modulation agent in the blood supply.

In another aspect, the invention further provides systems and kits for practicing the above described methods to treat the conditions, diseases or disorders (preferably but not limited to viral or bacterial infections).

In a different embodiment, a system in accordance with the invention may include: (1) an initiation energy source; and (2) one or more energy modulation agents. The system may further comprise (3) one or more activatable pharmaceutical agents. In an additional embodiment, the system may comprise only (1) the initiation energy source. In yet another embodiment, the system may comprise (1) an initiation energy source; and (3) one or more activatable pharmaceutical agents. FIG. 3 illustrates a system according to one exemplary embodiment of the invention. Referring to FIG. 3, an exemplary system according to one embodiment of the invention may have an initiation energy source 1 directed at the subject 4. An activatable pharmaceutical agent 2 and an energy modulation agent 3 are administered to the subject 4. The initiation energy source may additionally be controlled by a computer system 5 that is capable of directing the delivery of the initiation energy.

In preferred embodiments, the initiation energy source may be a linear accelerator equipped with image guided computer-control capability to deliver a precisely calibrated beam of radiation to a pre-selected coordinate. One example of such linear accelerators is the SmartBeam™ IMRT (intensity modulated radiation therapy) system from Varian medical systems (Varian Medical Systems, Inc., Palo Alto, Calif.).

In other embodiments, endoscopic or laproscopic devices equipped with appropriate initiation energy emitter may be used as the initiation energy source. In such systems, the initiation energy may be navigated and positioned at the pre-selected coordinate to deliver the desired amount of initiation energy to the site.

In further embodiments, dose calculation and robotic manipulation devices may also be included in the system.

In yet another embodiment, there is also provided a computer implemented system for designing and selecting suitable combinations of initiation energy source, energy transfer agent, and activatable pharmaceutical agent to treat the conditions, diseases or disorders (preferably but not limited to viral or bacterial infections), comprising:

a central processing unit (CPU) having a storage medium on which is provided:
a database of excitable compounds;
a first computation module for identifying and designing an excitable compound that is capable of binding with a target cellular structure or component; and
a second computation module predicting the different wavelengths of UV or visible or infrared irradiation to be emitted from at least one energy modulation agent in a vicinity of or within the target celluar structure,

wherein the system, upon selection of a target cellular structure or component, computes an excitable compound that is capable of binding with the target structure followed by a computation to predict the absorption energy of the excitable compound.

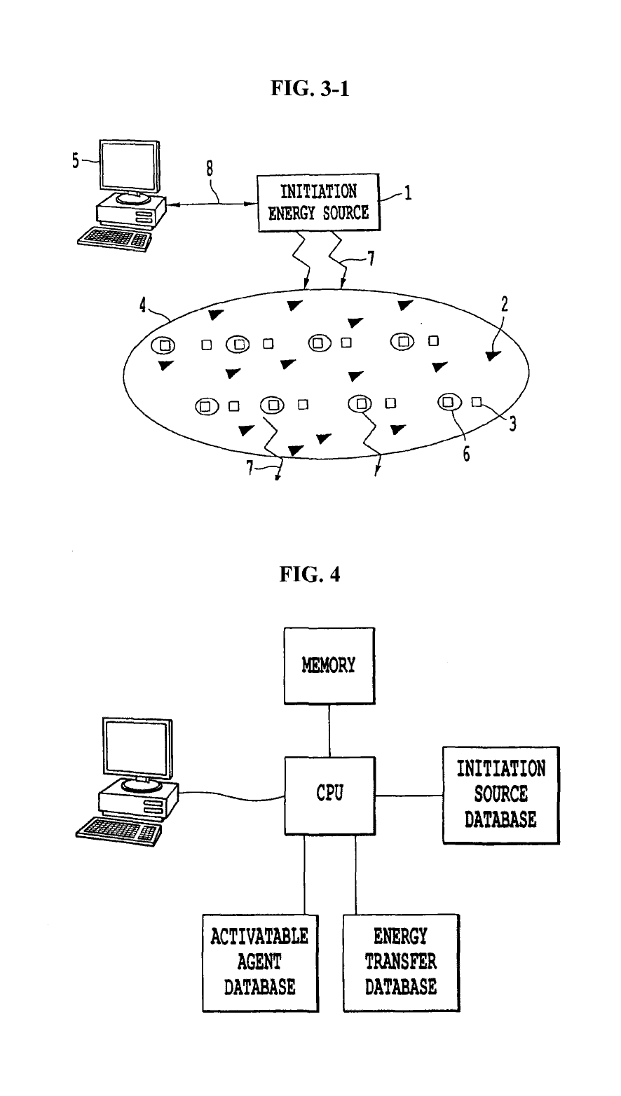

FIG. 4 illustrates an exemplary computer implemented system according to this embodiment of the invention. Referring to FIG. 4, an exemplary computer-implemented system according to one embodiment of the invention may have a central processing unit (CPU) connected to a memory unit, configured such that the CPU is capable of processing user inputs and selecting a combination of initiation source, activatable pharmaceutical agent, and energy transfer agent based on an energy spectrum comparison for use in a method of the invention.

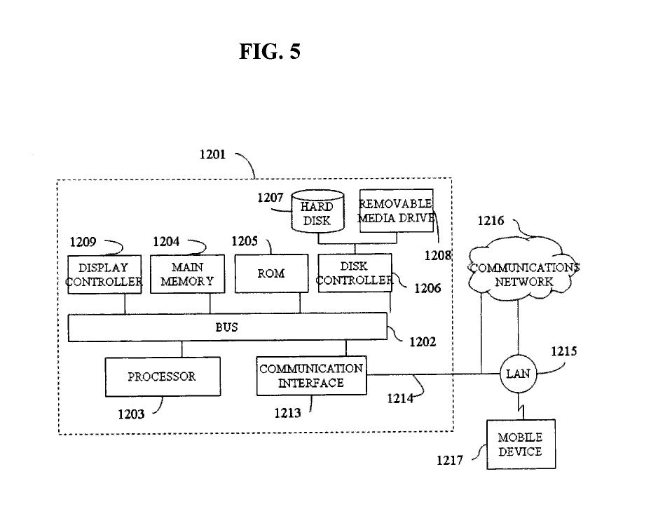

FIG. 5 illustrates a computer system 1201 for implementing various embodiments of the invention. The computer system 1201 may be used as the controller 55 to perform any or all of the functions described above. The computer system 1201 includes a bus 1202 or other communication mechanism for communicating information, and a processor 1203 coupled with the bus 1202 for processing the information. The computer system 1201 also includes a main memory 1204, such as a random access memory (RAM) or other dynamic storage device (e.g., dynamic RAM (DRAM), static RAM (SRAM), and synchronous DRAM (SDRAM)), coupled to the bus 1202 for storing information and instructions to be executed by processor 1203. In addition, the main memory 1204 may be used for storing temporary variables or other intermediate information during the execution of instructions by the processor 1203. The computer system 1201 further includes a read only memory (ROM) 1205 or other static storage device (e.g., programmable ROM (PROM), erasable PROM (EPROM), and electrically erasable PROM (EEPROM)) coupled to the bus 1202 for storing static information and instructions for the processor 1203.

The computer system 1201 also includes a disk controller 1206 coupled to the bus 1202 to control one or more storage devices for storing information and instructions, such as a magnetic hard disk 1207, and a removable media drive 1208 (e.g., floppy disk drive, read-only compact disc drive, read/write compact disc drive, compact disc jukebox, tape drive, and removable magneto-optical drive). The storage devices may be added to the computer system 1201 using an appropriate device interface (e.g., small computer system interface (SCSI), integrated device electronics (IDE), enhanced-IDE (E-IDE), direct memory access (DMA), or ultra-DMA).

The computer system 1201 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system 1201 may also include a display controller 1209 coupled to the bus 1202 to control a display 1210, such as a cathode ray tube (CRT), for displaying information to a computer user. The computer system includes input devices, such as a keyboard 1211 and a pointing device 1212, for interacting with a computer user and providing information to the processor 1203. The pointing device 1212, for example, may be a mouse, a trackball, or a pointing stick for communicating direction information and command selections to the processor 1203 and for controlling cursor movement on the display 1210. In addition, a printer may provide printed listings of data stored and/or generated by the computer system 1201.

The computer system 1201 performs a portion or all of the processing steps of the invention (such as for example those described in relation to FIG. 5) in response to the processor 1203 executing one or more sequences of one or more instructions contained in a memory, such as the main memory 1204. Such instructions may be read into the main memory 1204 from another computer readable medium, such as a hard disk 1207 or a removable media drive 1208. One or more processors in a multi-processing arrangement may also be employed to execute the sequences of instructions contained in main memory 1204. In alternative embodiments, hard-wired circuitry may be used in place of or in combination with software instructions. Thus, embodiments are not limited to any specific combination of hardware circuitry and software.

As stated above, the computer system 1201 includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, or other physical medium, a carrier wave (described below), or any other medium from which a computer can read.

Stored on any one or on a combination of computer readable media, the invention includes software for controlling the computer system 1201, for driving a device or devices for implementing the invention, and for enabling the computer system 1201 to interact with a human user (e.g., print production personnel). Such software may include, but is not limited to, device drivers, operating systems, development tools, and applications software. Such computer readable media further includes the computer program product of the invention for performing all or a portion (if processing is distributed) of the processing performed in implementing the invention.

The computer code devices of the invention may be any interpretable or executable code mechanism, including but not limited to scripts, interpretable programs, dynamic link libraries (DLLs), Java classes, and complete executable programs. Moreover, parts of the processing of the invention may be distributed for better performance, reliability, and/or cost.

The term “computer readable medium” as used herein refers to any medium that participates in providing instructions to the processor 1203 for execution. A computer readable medium may take many forms, including but not limited to, non-volatile media, volatile media, and transmission media. Non-volatile media includes, for example, optical, magnetic disks, and magneto-optical disks, such as the hard disk 1207 or the removable media drive 1208. Volatile media includes dynamic memory, such as the main memory 1204. Transmission media includes coaxial cables, copper wire and fiber optics, including the wires that make up the bus 1202. Transmission media also may also take the form of acoustic or light waves, such as those generated during radio wave and infrared data communications.

Various forms of computer readable media may be involved in carrying out one or more sequences of one or more instructions to processor 1203 for execution. For example, the instructions may initially be encoded on a magnetic disk of a remote computer. The remote computer can load the instructions for implementing all or a portion of the invention remotely into a dynamic memory and send the instructions over a telephone line using a modem. A modem local to the computer system 1201 may receive the data on the telephone line and use an infrared transmitter to convert the data to an infrared signal. An infrared detector coupled to the bus 1202 can receive the data carried in the infrared signal and place the data on the bus 1202. The bus 1202 carries the data to the main memory 1204, from which the processor 1203 retrieves and executes the instructions. The instructions received by the main memory 1204 may optionally be stored on storage device 1207 or 1208 either before or after execution by processor 1203.

The computer system 1201 also includes a communication interface 1213 coupled to the bus 1202. The communication interface 1213 provides a two-way data communication coupling to a network link 1214 that is connected to, for example, a local area network (LAN) 1215, or to another communications network 1216 such as the Internet. For example, the communication interface 1213 may be a network interface card to attach to any packet switched LAN. As another example, the communication interface 1213 may be an asymmetrical digital subscriber line (ADSL) card, an integrated services digital network (ISDN) card or a modem to provide a data communication connection to a corresponding type of communications line. Wireless links may also be implemented. In any such implementation, the communication interface 1213 sends and receives electrical, electromagnetic or optical signals that carry digital data streams representing various types of information.

The network link 1214 typically provides data communication through one or more networks to other data devices. For example, the network link 1214 may provide a connection to another computer through a local network 1215 (e.g., a LAN) or through equipment operated by a service provider, which provides communication services through a communications network 1216. The local network 1214 and the communications network 1216 use, for example, electrical, electromagnetic, or optical signals that carry digital data streams, and the associated physical layer (e.g., CAT 5 cable, coaxial cable, optical fiber, etc). The signals through the various networks and the signals on the network link 1214 and through the communication interface 1213, which carry the digital data to and from the computer system 1201 maybe implemented in baseband signals, or carrier wave based signals. The baseband signals convey the digital data as unmodulated electrical pulses that are descriptive of a stream of digital data bits, where the term “bits” is to be construed broadly to mean symbol, where each symbol conveys at least one or more information bits. The digital data may also be used to modulate a carrier wave, such as with amplitude, phase and/or frequency shift keyed signals that are propagated over a conductive media, or transmitted as electromagnetic waves through a propagation medium. Thus, the digital data may be sent as unmodulated baseband data through a “wired” communication channel and/or sent within a predetermined frequency band, different than baseband, by modulating a carrier wave. The computer system 1201 can transmit and receive data, including program code, through the network(s) 1215 and 1216, the network link 1214, and the communication interface 1213. Moreover, the network link 1214 may provide a connection through a LAN 1215 to a mobile device 1217 such as a personal digital assistant (PDA) laptop computer, or cellular telephone.

The reagents and chemicals useful for methods and systems of the invention may be packaged in kits to facilitate application of the invention to treat the conditions, diseases or disorders (preferably but not limited to viral or bacterial infections). In one exemplary embodiment, a kit includes a psoralen or an alkylating agent. A further embodiment of kit would comprise at least one activatable pharmaceutical agent capable of causing a predetermined cellular change upon exposure of a target structure to one or more wavelengths of UV or visible or infrared irradiation emitted from at least one energy modulation agent in a vicinity of or within the target structure, at least one energy modulation agent capable of activating the at least one activatable agent when energized, and containers suitable for storing the agents in stable form, and preferably further comprising instructions for administering the at least one activatable pharmaceutical agent and at least one energy modulation agent to a subject, and for applying an initiation energy from an initiation energy source to activate the activatable pharmaceutical agent. The instructions could be in any desired form, including but not limited to, printed on a kit insert, printed on one or more containers, as well as electronically stored instructions provided on an electronic storage medium, such as a computer readable storage medium. Also optionally included is a software package on a computer readable storage medium that permits the user to integrate the information and calculate a control dose, to calculate and control intensity of the irradiation source (e.g., one of the x-ray sources noted herein).

In different aspect of the invention, a kit for modifying a target structure which mediates or is associated with a biological activity, comprising:

at least one agent selected from the group consisting of energy modulation agents, photoactivatable agents (such as the psoralen, a coumarin, or an alkylating agent or a derivative thereof), plasmonics-active agents and combinations thereof;
wherein the energy modulation agent, if present, upgrades or downgrades an initiation energy to an activation energy capable of causing, either directly or indirectly, a predetermined change in the target structure upon exposure of the target structure to different wavelengths of UV or visible or infrared irradiation emitted from at least one energy modulation agent in a vicinity of or within the target structure;
wherein the plasmonics-active agent, if present, enhances or modifies the applied initiation energy or the activation energy generated by the energy modulation agent, or both; and

one or more containers suitable for storing the agents in stable forms.

In a different embodiment, a kit for performing a condition, disorder or disease treatment, comprises:

a psoralen, a coumarin, or an alkylating agent or a derivative thereof;

at least one energy modulation agent capable of adsorbing, intensifying or modifying an initiation energy into an energy that is capable of causing a predetermined change in a target structure upon exposure of a target structure to different wavelengths of UV or visible or infrared irradiation emitted from at least one energy modulation agent in a vicinity of or within the target structure; and

containers suitable for storing the above-noted agents in stable form.

In yet another embodiment, the kit may further comprise instructions for administering the at least one energy modulation agent and/or the photoactivatable agent to a subject.

In one embodiment of the invention, electromagnetic enhancements are used. These enhancements are divided into two main classes: a) enhancements that occur only in the presence of a radiation field, and b) enhancements that occur even without a radiation field. The first class of enhancements is further divided into several processes. Plasma resonances on the substrate surfaces, also called surface plasmons, provide a major contribution to electromagnetic enhancement. An effective type of plasmonics-active substrate comprises nanostructured metal particles, protrusions, or rough surfaces of metallic materials. Incident light irradiating these surfaces excites conduction electrons in the metal, and induces excitation of surface plasmons leading to Raman/luminescence enhancement. At the plasmon frequency, the metal nanoparticles (or nanostructured roughness) become polarized, resulting in large field-induced polarizations and thus large local fields on the surface. These local fields increase the luminescence/Raman emission intensity, which is proportional to the square of the applied field at the molecule. As a result, the effective electromagnetic field experienced by the analyte molecule on these surfaces is much larger than the actual applied field. This field decreases as l/r³ away from the surface. Therefore, in the electromagnetic models, the luminescence/Raman-active analyte molecule is not required to be in contact with the metallic surface but can be located anywhere within the range of the enhanced local field, which can polarize this molecule. The dipole oscillating at the wavelength A of Raman or luminescence can, in turn, polarize the metallic nanostructures and, ifl is in resonance with the localized surface plasmons, the nanostructures can enhance the observed emission light (Raman or luminescence).

There are two main sources of electromagnetic enhancement: (1) first, the laser electromagnetic field is enhanced due to the addition of a field caused by the polarization of the metal particle; (2) in addition to the enhancement of the excitation laser field, there is also another enhancement due to the molecule radiating an amplified Raman/luminescence field, which further polarizes the metal particle, thereby acting as an antenna to further amplify the Raman/luminescence signal. Plasmonics-active metal nanoparticles also exhibit strongly enhanced visible and near-infrared light absorption, several orders of magnitude more intense compared to conventional laser phototherapy agents. The use of plasmonic nanoparticles as highly enhanced photoabsorbing agents thus provides a selective and efficient phototherapy strategy.

Experimental evidence suggests that the origin of the 10⁶- to 10¹⁵-fold Raman enhancement primarily arises from two mechanisms: a) an electromagnetic “lightning rod” effect occurring near metal surface structures associated with large local fields caused by electromagnetic resonances, often referred to as “surface plasmons”; and b) a chemical effect associated with direct energy transfer between the molecule and the metal surface.

According to classical electromagnetic theory, electromagnetic fields can be locally amplified when light is incident on metal nanostructures. These field enhancements can be quite large (typically 10⁶- to 10⁷-fold, but up to 10⁵-fold enhancement at “hot spots”). When a nanostructured metallic surface is irradiated by an electromagnetic field (e.g., a laser beam), electrons within the conduction band begin to oscillate at a frequency equal to that of the incident light. These oscillating electrons, called “surface plasmons,” produce a secondary electric field which adds to the incident field. If these oscillating electrons are spatially confined, as is the case for isolated metallic nanospheres or roughened metallic surfaces (nanostructures), there is a characteristic frequency (the plasmon frequency) at which there is a resonant response of the collective oscillations to the incident field. This condition yields intense localized field enhancements that can interact with molecules on or near the metal surface. In an effect analogous to a “lightning rod,” secondary fields are typically most concentrated at points of high curvature on the roughened metal surface.

Other Applications

Referring to FIG. 3-1, an exemplary system according to one embodiment of the invention may have an initiation energy source 1 directed at a generic medium 4 (biological or non-biological). Activatable agents 2 (e.g., photoactivatable agents) and an energy modulation agents 3 (such as the energy modulation agents and/or the other energy modulation agents described above) are dispersed throughout the medium 4. The initiation energy source 1 may additionally be connected via a network 8 to a computer system 5 capable of directing the delivery of the initiation energy. In various embodiments, the energy modulation agents 3 are encapsulated energy modulation agents 6, depicted in FIG. 3-1 as silica encased energy modulation agents. As shown in FIG. 3-1, initiation energy 7 in the form of radiation from the initiation energy source 1 permeated throughout the medium 4. A more thorough discussion of the computer system 5 is provided below in reference to FIGS. 4 and 5. The initiation energy source 1 can be an external energy source or an energy source located at least partially in the medium 4. Activatable agents 2 and/or the energy modulation agents 3 can include plasmonics agents which enhance either the applied energy or the energy emitted from the energy modulation agents 3 so as to directly or indirectly produce a change in the medium.

In various embodiments of this invention regardless of the specific medical or non-medical use, the initiation energy source 1 may be a linear accelerator equipped with image guided computer-control capability to deliver a precisely calibrated beam of radiation to a pre-selected coordinate. One example of such linear accelerators is the SmartBeam™ IMRT (intensity modulated radiation therapy) system from Varian medical systems (Varian Medical Systems, Inc., Palo Alto, Calif.). In other embodiments, the initiation energy source 1 may be commercially available components of X-ray machines or non-medical X-ray machines. X-ray machines that produce from 10 to 150 keV X-rays are readily available in the marketplace. For instance, the General Electric Definium series or the Siemens MULTIX series are but two examples of typical X-ray machines designed for the medical industry, while the Eagle Pack series from Smith Detection is an example of a non-medical X-ray machine. As such, the invention is capable of performing its desired function when used in conjunction with commercial X-ray equipment.

In other embodiments, the initiation energy source 1 can be a radio frequency or microwave source emitting radio waves at a frequency which permeates the medium and which triggers or produces secondary radiant energy emission within the medium by interaction with the energy modulation elements 6 therein. In other embodiments, the initiation energy source 1 can be an ultraviolet, visible, near infrared (NIR) or infrared (IR) emitter emitting at a frequency which permeates the medium 4 and which triggers or produces secondary radiant energy emission within medium 4 by interaction with the energy modulation elements 6 therein.

The energy modulation structures of this invention can be provided on the interior of sealed quartz or glass tubes or can be provided coated on the surface of spheres or tubes, and further encapsulated with a silicate or another passivation layer. It is known that ultraviolet (UV) with a wavelength of 254 nm tends to inactivate most types of microorganisms. The deep UV diamond emission lines make diamond (and DLC) a suitable choice for inclusion as or with the other energy modulation structures described herein or as a sole part of the energy modulation structures of this invention.

A mediums to be sterilized by the present invention can include food products, medical products and cosmetic products.

U.S. Pat. No. 6,087,141 (the entire contents of which are incorporated herein by reference) describes an ultraviolet light activated psoralen process for sterilization of blood transfusion products. The invention can be applied for the neutralization of AIDS and HIV or EBOLA or other viral or pathogenic agents in blood transfusion products. In this embodiment, at least one photoactivatable agent is selected from psoralens, an alkylating agent, INA, pyrene cholesteryloleate, acridine, porphyrin, fluorescein, rhodamine, 16-diazorcortisone, ethidium, transition metal complexes of bleomycin, transition metal complexes of deglycobleomycin organoplatinum complexes, alloxazines, vitamin Ks, vitamin L, vitamin metabolites, vitamin precursors, naphthoquinones, naphthalenes, naphthols and derivatives thereof having planar molecular conformations, porphorinporphyrins, dyes and phenothiazine derivatives, coumarins, quinolones, quinones, anthroquinones, porphycene, rubyrin, rosarin, hexaphyrin, sapphyrin, chlorophyl, chlorin, phthalocynine, porphyrazine, bacteriochlorophyl, pheophytin, texaphyrin macrocyclic-based component, or a metalated derivative thereof. These photoactivatable agents serve as recipients for the secondarily generated light induced by the down conversion or upconversion from the energy modulation agents.

In various embodiments of the invention, the UV or visible light recipients are secondary agents performing other functions. Suitable secondary agents for the invention include secondary emitters, cytotoxic agents, magnetic resonance imaging (MRI) agents, positron emission tomography (PET) agents, radiological imaging agents, or photodynamic therapy (PDT) agents.

These photoactivatable agents (recipients and secondary agents) are introduced into the blood product (or a patient's blood stream). An initiation energy such as x-ray (or other high energy source, deep UV, electrons, gamma rays) is applied to the blood product (or to the patient). The energy modulation structures of this invention (either included in the blood product or in encapsulated structures) generate secondary light such as UV or visible light which activates the photoactivatable agents in the blood products.

In a specific example, the photoactivatable agent is a psoralen, a coumarin, or an alkylating agent or a derivative thereof, and one can sterilize blood products in vivo (i.e., in a patient) or in a container of the blood product (such as for example donated blood). The treatment can be applied to treat disorders such as for example a cancer cell, a tumor cell, an autoimmune deficiency symptom virus, or a blood-borne germicide, or virus.

Generalized Statements

The following numbered statements describe different aspects of this invention and are not intended to limit the invention. Indeed, in the invention, various of these aspects can be combined in any order with other aspects set forth in this section and in the subject matter of the specification.

Statement 1. A method for treating a subject carrying a virus or a bacterium, comprising: providing within the subject 1) one or more light emitters capable of emitting at least one wavelength of light and 2) at least one photoactivatable drug for treatment of the subject carrying the virus or the bacterium; applying initiation energy from at least one source to a target inside the subject to activate the light emitters; from said at least one wavelength of light, activating inside the subject the at least one photoactivatable drug; and inside the subject, reacting the activated photoactivatable drug with the virus or the bacterium to inactivate the virus or the bacterium to thereby treat the subject. In this aspect of the invention, the photoactivatable drug in a preferred embodiment can be a DNA intercalator such as psoralen or an alkylating agent. In this aspect of the invention, x-ray energy can be used as a preferred initiation energy source in conjunction with an energy modulation agent to produce UV or visible light internally in the subject nearby or at a diseased site.

Statement 2. The method of statement 1, further comprising generating in vivo a vaccine against the virus.

Statement 3. The method of statement 1, wherein activating inside the subject the at least one photoactivatable drug comprises bonding the photoactivatable drug to a cellular structure.

Statement 4. The method of statement 2, wherein the bonding comprises at least one of 1) bonding the photoactivatable drug to at least one of nuclear DNA, mRNA, rRNA, ribosome, mitochondrial DNA and 2) bonding the photoactivatable drug to lipid bilayers of a virus.

Statement 5. The method of statement 2, wherein the bonding comprises bonding the photoactivatable drug to lipid bilayers of at least one virus selected from the group consisting of an ebola virus, an encephalitis virus, a West Nile virus, and an HIV virus.

Statement 6. The method of statement 1, further comprising activating inside the subject the at least one photoactivatable drug comprises activating a psoralen.

Statement 7. The method of statement 1, further comprising activating inside the subject the at least one photoactivatable drug comprises activating 8 MOP or AMT.

Statement 8. The method of statement 1, wherein activating inside the subject the at least one photoactivatable drug comprises activating an alkylating agent.

Statement 9. The method of statement 1, wherein activating inside the subject the at least one photoactivatable drug comprises activating 1,5-iodonophthylazide.

Statement 10. The method of statement 1, wherein activating inside the subject the at least one photoactivatable drug comprises activating a drug for treating the virus.

Statement 11. The method of statement 1, wherein activating inside the subject the at least one photoactivatable drug comprises activating a drug for treating the bacterium.

Statement 12. The method of statement 1, wherein said generating said at least one wavelength of light comprises generating at least two different wavelengths of light, each wavelength of light associated with a different biological response.

Statement 13. The method of statement 8, further comprising

activating plural biological responses inside the subject depending on the different wavelengths of light provided internally within the subject, wherein the different wavelengths activate different biological responses

Statement 14. The method of statement 1, wherein activating the at least one photoactivatable drug comprises activating a first biological response with a first wavelength of light and a second biological response with a second wavelength of light.

Statement 15. The method of statement 14, wherein activating a second biological response comprises generating a reactive oxygen species.

Statement 16. The method of statement 14, wherein activating a first biological response comprises at least one of photoactivating a drug, sterilizing the target structure, photoactivating a psoralen, photoactivating iodonophthylazide, generating a reactive oxygen species, inducing an autoimmune response, exciting a DNA strand of a cancer cell, redirecting a metabolic pathway, up-regulating genes, down-regulating genes, secreting cytokines, altering cytokine receptor responses, releasing metabolites, generating a vaccine, or a combination thereof.

Statement 17. The method of statement 14, wherein activating a second biological response comprises at least one of photoactivating a drug, sterilizing the target structure, photoactivating a psoralen, photoactivating iodonophthylazide, generating a reactive oxygen species, inducing an autoimmune response, exciting a DNA strand of a cancer cell, redirecting a metabolic pathway, up-regulating genes, down-regulating genes, secreting cytokines, altering cytokine receptor responses, releasing metabolites, generating a vaccine, or a combination thereof.

Statement 18. The method of statement 14, wherein at least one of said activating a first biological response and said activating a second biological response comprises altering a cellular response or a metabolic rate of the target structure.

Statement 19. The method of statement 14, wherein activating a first biological response comprises emitting ultraviolet light to act as a germicide, and wherein activating a second biological response comprises emitting near infrared light to act as an anti-inflammatory.

Statement 20. The method of statement 14, wherein activating a first biological response comprises emitting ultraviolet light to act as a germicide, and wherein activating a second biological response comprises emitting near infrared light to promote cellular proliferation.

Statement 21. The method of statement 14, wherein activating a first biological response comprises emitting ultraviolet light to act as a germicide, and wherein activating a second biological response comprises emitting near infrared light to reduce pain.

Statement 22. The method of statement 14, wherein activating a first biological response comprises photactivating a pharmaceutical agent, and wherein activating a second biological response comprises heating a local area of the target structure.

Statement 23. The method of statement 1, wherein providing within the subject one or more light emitters comprises administering to said subject as the one or more light emitters at least one energy modulation agent which adsorbs, intensifies or modifies said initiation energy.

Statement 24. The method of statement 23, wherein said energy modulation agent comprises at least one of a biocompatible fluorescing metal nanoparticle, fluorescing metal oxide nanoparticle, fluorescing metal coated metal oxide nanoparticle, fluorescing dye molecule, gold nanoparticle, silver nanoparticle, gold-coated silver nanoparticle, a water soluble quantum dot encapsulated by polyamidoamine dendrimers, a luciferase, a biocompatible phosphorescent molecule, a combined electromagnetic energy harvester molecule, and a lanthanide chelate exhibiting intense luminescence.

Statement 25. The method of statement 23, wherein said energy modulation agent comprises a down-converting agent.

Statement 26. The method of statement 25, wherein said energy modulation agent comprises inorganic materials selected from the group consisting of: metal oxides; metal sulfides; doped metal oxides; and mixed metal chalcogenides.

Statement 27. The method of statement 25, wherein said energy modulation agent comprises at least one of Y₂O₃, Y₂O₂S, NaYF₄, NaYbF₄, YAG, YAP, Nd₂O₃, LaF₃, LaCl₃, La₂O₃, TiO₂, LuPO₄, YVO₄, YbF₃, YF₃, Na-doped YbF₃, ZnS; ZnSe; MgS; CaS, CaWO₄, CaSiO₂:Pb, and alkali lead silicate including compositions of SiO₂, B₂O₃, Na₂O, K₂O, PbO, MgO, or Ag, and combinations or alloys or layers thereof.

Statement 28. The method of statement 25, wherein said energy modulation agent comprises at least one of ZnSeS:Cu, Ag, Ce, Tb; CaS: Ce,Sm; La₂O₂S:Tb; Y₂O₂S:Tb; Gd₂O₂S:Pr, Ce, F; LaPO₄.

Statement 29. The method of statement 25, wherein said energy modulation agent comprises at least one of ZnS:Ag, ZnS:Cu, Pb, and alloys of the ZnSeS.

Statement 30. The method of statement 25, wherein said energy modulation agent comprises at least one of sodium yttrium fluoride (NaYF₄), lanthanum fluoride (LaF₃), lanthanum oxysulfide (La₂O₂S), yttrium oxysulfide (Y₂O₂S), yttrium fluoride (YF₃), yttrium gallate, yttrium aluminum garnet (YAG), gadolinium fluoride (GdF₃), barium yttrium fluoride (BaYF₅, BaY₂F₈), gadolinium oxysulfide (Gd₂O₂S), calcium tungstate (CaWO₄), yttrium oxide:terbium (Yt₂O₃Tb), gadolinium oxysulphide:europium (Gd₂O₂S:Eu), lanthanum oxysulphide:europium (La₂O₂S:Eu), and gadolinium oxysulphide:promethium, cerium, fluorine (Gd₂O₂S:Pr,Ce,F), YPO₄:Nd, LaPO₄:Pr, (Ca,Mg)SO₄:Pb, YBO₃:Pr, Y₂SiO₅:Pr, Y₂Si₂O₇:Pr, SrLi₂SiO₄:Pr,Na, and CaLi₂SiO₄:Pr.

Statement 31. The method of statement 25, wherein said energy modulation agent comprises at least one of KSrPO₄:Eu²⁺, Pr³⁺, NaGdF₄:Eu, Zn,SiO:Tb³⁺, Yb³⁺, β-NaGdF₄ co-doped with Ce³⁺ and Tb³⁺ ions, and Gd₂O₂S:Tm or BaYF₅:Eu³⁺.

Statement 32. The method of statement 23, wherein said energy modulation agent comprises an up converting agent.

Statement 33. The method of statement 32, wherein said energy modulation agent at least one of Y₂O₃, Y₂O₂S, NaYF₄, NaYbF₄, YAG, YAP, Nd₂O₃, LaF₃, LaCl₃, La₂O₃, TiO₂, LuPO₄, YVO₄, YbF₃, YF₃, Na-doped YbF₃, or SiO₂ or alloys or layers thereof.

Statement 34. The method of statement 1, further comprising producing a change which results in destruction, lysis or inactivation of the virus.

Statement 35. The method of statement 1, further comprising producing a change which results in wound healing.

Statement 36. The method of statement 35, further comprising producing a change which results in enhancement of tissue growth, nerve regeneration or sensory regeneration/restoration.

Statement 37. The method of statement 35, further comprising producing a change which results in a treatment of a prion, viral, bacterial, fungal, or parasitic infection.

Statement 38. The method of statement 1, wherein the initiation energy is UV radiation, visible light, IR radiation, x-rays, gamma rays, an electron beam, microwaves or radio waves.

Statement 39. The method of statement 1, wherein the at least one wavelength of the light is provided internally within the subject.

Statement 40. The method of statement 39, wherein the at least one wavelength of the light provided internally within the subject is generated from a distal end of a catheter inserted into the subject.

Statement 41. The method of statement 40, wherein the at least one wavelength of the light provided internally within the subject is generated from energy modulation agents disposed at the distal end of the catheter.

Statement 42. The method of statement 41, wherein the at least one wavelength of the light provided internally within the subject is generated from down converting materials disposed at the distal end of the catheter.

Statement 43. The method of statement 42, wherein the at least one wavelength of the light provided internally within the subject is generated from up converting materials disposed at the distal end of the catheter.

Statement 44. The method of statement 1, further comprising providing a plasmonics-active agent which enhances or modifies the initiation energy or said at least one wavelength of light.

Statement 45. The method of statement 44, wherein the plasmonics-active agent comprises metal nanostructures.

Statement 46. The method of statement 45, wherein the metal nanostructures are nanospheres, nanorods, nanocubes, nanopyramids, nanoshells, multi-layer nanoshells and combinations thereof.

Statement 47. The method of statement 1, wherein the initiation energy comprises at least one or more of x-rays, gamma rays, an electron beam, UV radiation, visible light, or infrared radiation.

Statement 48. The method of statement 1, further comprising treating with said at least one wavelength of light at least one condition selected from the group consisting of cancer, bacterial infection, parasitic infection, prion infection, fungal infection, immune rejection response, autoimmune disorder, and aplastic condition.

Statement 49. The method of statement 1, further comprising treating with said at least one wavelength of light a condition, a disorder, or a disease selected from the group consisting of cardiac ablasion, photoangioplastic condition, intimal hyperplasia, arteriovenous fistula, macular degeneration, psoriasis, acne, hopecia areata, portwine spots, hair removal, autoimmune diseases, rheumatoid and inflammatory arthritis, behavioral and cognitive disorder/condition, joint condition, Parkinson's disease, retinal injury and other ocular diseases, enlarged prostate, varicose veins, reduction or removal of fat deposits (liposuction), nerve regeneration, sensory regeneration/restoration, wound healing, chronic pain, conditions occurring in bone tissue, conditions occurring in a soft tissue and/or cartilage, and lymph node condition.

Statement 50. The method of statement 1, wherein the at least one photoactivatable drug comprise at least one pharmaceutical agent selected from the group consisting of a psoralen, pyrene cholesteryloleate, acridine, porphyrin, fluorescein, rhodamine, 16-diazorcortisone, ethidium, transition metal complexes of bleomycin, transition metal complexes of deglycobleomycin organoplatinum complexes, alloxazines, vitamin Ks, vitamin L, vitamin metabolite, vitamin precursor, naphthoquinone, naphthalene, naphthol and derivatives thereof having planar molecular conformations, porphorinporphyrin, dye and phenothiazine derivative, coumarin, quinolone, quinone, and anthroquinone.

Statement 51. The method of statement 1, wherein the at least one photoactivatable drug comprises one or more of a psoralen, a coumarin, a porphyrin, and iodonophthylazide, or a derivative thereof.

Statement 52. The method of statement 1, wherein the at least one photoactivatable drug comprises at least one pharmaceutical agent selected from the group consisting of 7,8-dimethyl-10-ribityl, isoalloxazine, 7,8,10-trimethylisoalloxazine, 7,8-dimethylalloxazine, isoalloxazine-adenine dinucleotide, alloxazine mononucleotide, aluminum (III) phthalocyanine tetrasulonate, hematophorphyrin, and phthadocyanine.

Statement 53. The method of statement 1, wherein the one or more light emitters comprise at least one of a diamond light emitter and a diamond-like carbon light emitter.

Statement 54. The method of statement 1, wherein the one or more light emitters comprise one or more coated energy modulation agents.

Statement 55. The method of statement 1, wherein the one or more coated energy modulation agents have a biocompatible coating.

Statement 56. The method of statement 65, wherein the biocompatible coating comprises poly(esters) based on polylactide (PLA), polyglycolide (PGA), polycarpolactone (PCL), poly(hydroxyalkanoate) of the PHB-PHV class, poly(ester), natural polymer, modified poly(saccharide), starch, cellulose, chitosan, polyethylene oxide, poly(ether)(ester) block copolymers, ethylene vinyl acetate copolymer or a combination thereof.

Statement 57. The method of statement 1, wherein the biocompatible coating comprises at least one of a silica, a silicate, nano-diamond film, a diamond like carbon coating, and a graphene material.

Statement 58. The method of statement 1, wherein the at least one photoactivatable drug comprises an alkylating agent and psoralen.

Statement 59. The method of statement 1, wherein the at least one photoactivatable drug comprises at least one photoactivatable hydrophobic compound of the following formula I:

X—Ar—Y I,

wherein Ar is a hydrophobic moiety, and X and Y are each independently hydrogen or a reactive group, provided that at least one of X or Y is a reactive group.

Statement 60. A method for treating a subject carrying a virus or a bacterium, the method comprising: providing within the subject at least one photoactivatable drug for treatment of the virus or the bacterium; applying initiation energy from at least one source to a target inside the subject; activating directly or indirectly the at least one photoactivatable drug at the target inside the subject; and inside the subject, reacting the activated photoactivatable drug with the virus or bacterium to inactivate the virus or the bacterium, thereby treating the subject. In this aspect of the invention, the photoactivatable drug in a preferred embodiment can be a DNA intercalator such as psoralen or an alkylating agent. In this aspect of the invention, x-ray energy can be used as a preferred initiation energy source in conjunction with an energy modulation agent to produce UV or visible light internally in the subject nearby or at a diseased site. Furthermore, any of the aspects defined in statements 1-59 can be used here (individually or in combination) in this treatment method defined by statement 60.

Statement 61. A method for treating a subject carrying a virus or a bacterium, the method comprising: providing within a respiratory track of the subject at least one photoactivatable drug for treatment of the virus or the bacterium; applying initiation energy from at least one source to the respiratory track; activating directly or indirectly the at least one photoactivatable drug at the target inside the respiratory track; and inside the respiratory track, reacting the activated photoactivatable drug with the virus or bacterium to inactivate the virus or the bacterium, thereby treating the subject. In this aspect of the invention, the photoactivatable drug in a preferred embodiment can be a DNA intercalator such as psoralen or an alkylating agent. In this aspect of the invention, x-ray energy can be used as a preferred initiation energy source in conjunction with an energy modulation agent to produce UV or visible light internally in the subject nearby or at a diseased site. Furthermore, any of the aspects defined in statements 1-59 can be used here (individually or in combination) in this treatment method defined by statement 61.

Statement 62. A method for treating a subject carrying a virus or a bacterium, the method comprising: providing within lymph nodes of the subject at least one photoactivatable drug for treatment the subject carrying the virus or the bacterium; applying initiation energy from at least one source to the lymph nodes; activating directly or indirectly the at least one photoactivatable drug at the target inside the lymph nodes; and inside the lymph nodes, reacting the activated photoactivatable drug with the virus or bacterium to inactivate the virus or the bacterium, thereby treating the subject. In this aspect of the invention, the photoactivatable drug in a preferred embodiment can be a DNA intercalator such as psoralen or an alkylating agent. In this aspect of the invention, x-ray energy can be used as a preferred initiation energy source in conjunction with an energy modulation agent to produce UV or visible light internally in the subject nearby or at the lymph nodes. Furthermore, any of the aspects defined in statements 1-59 can be used here (individually or in combination) in this treatment method defined by statement 62.

Additional modifications and variations of the invention are possible in light of the above teachings. It is therefore to be understood that within the scope of the appended claims, the invention may be practiced otherwise than as specifically described herein.

1: A method for treating a subject carrying a virus or a bacterium, comprising:
providing within the subject 1) one or more light emitters capable of emitting at least one wavelength of light and 2) at least one photoactivatable drug for treatment of the subject carrying the virus or the bacterium;
applying initiation energy from at least one source to a target inside the subject to activate the light emitters;
from said at least one wavelength of light, activating inside the subject the at least one photoactivatable drug; and
inside the subject, reacting the activated photoactivatable drug with the virus or the bacterium to inactivate the virus or the bacterium to thereby treat the subject.

2: The method of claim 1, further comprising generating in vivo a vaccine against the virus.

3: The method of claim 1, wherein activating inside the subject the at least one photoactivatable drug comprises bonding the photoactivatable drug to a cellular structure.

4: The method of claim 2, wherein the bonding comprises at least one of 1) bonding the photoactivatable drug to at least one of nuclear DNA, mRNA, rRNA, ribosome, mitochondrial DNA and 2) bonding the photoactivatable drug to lipid bilayers of a virus.

5: The method of claim 2, wherein the bonding comprises bonding the photoactivatable drug to lipid bilayers of at least one virus selected from the group consisting of an ebola virus, an encephalitis virus, a West Nile virus, and an HIV virus.

6: The method of claim 1, further comprising activating inside the subject the at least one photoactivatable drug comprises activating a psoralen.

7: The method of claim 1, further comprising activating inside the subject the at least one photoactivatable drug comprises activating 8 MOP or AMT.

8: The method of claim 1, wherein activating inside the subject the at least one photoactivatable drug comprises activating an alkylating agent.

9: The method of claim 1, wherein activating inside the subject the at least one photoactivatable drug comprises activating 1,5-iodonophthylazide.

10: The method of claim 1, wherein activating inside the subject the at least one photoactivatable drug comprises activating a drug for treating the virus.

11: The method of claim 1, wherein activating inside the subject the at least one photoactivatable drug comprises activating a drug for treating the bacterium.

12: The method of claim 1, wherein said generating said at least one wavelength of light comprises generating at least two different wavelengths of light, each wavelength of light associated with a different biological response.

13: The method of claim 8, further comprising
activating plural biological responses inside the subject depending on the different wavelengths of light provided internally within the subject, wherein the different wavelengths activate different biological responses

14: The method of claim 1, wherein activating the at least one photoactivatable drug comprises activating a first biological response with a first wavelength of light and a second biological response with a second wavelength of light.

15: The method of claim 14, wherein activating a second biological response comprises generating a reactive oxygen species.

16: The method of claim 14, wherein activating a first biological response comprises at least one of photoactivating a drug, sterilizing the target structure, photoactivating a psoralen, photoactivating iodonophthylazide, generating a reactive oxygen species, inducing an autoimmune response, exciting a DNA strand of a cancer cell, redirecting a metabolic pathway, up-regulating genes, down-regulating genes, secreting cytokines, altering cytokine receptor responses, releasing metabolites, generating a vaccine, or a combination thereof.

17: The method of claim 14, wherein activating a second biological response comprises at least one of photoactivating a drug, sterilizing the target structure, photoactivating a psoralen, photoactivating iodonophthylazide, generating a reactive oxygen species, inducing an autoimmune response, exciting a DNA strand of a cancer cell, redirecting a metabolic pathway, up-regulating genes, down-regulating genes, secreting cytokines, altering cytokine receptor responses, releasing metabolites, generating a vaccine, or a combination thereof.

18: The method of claim 14, wherein at least one of said activating a first biological response and said activating a second biological response comprises altering a cellular response or a metabolic rate of the target structure.

19: The method of claim 14, wherein activating a first biological response comprises emitting ultraviolet light to act as a germicide, and wherein activating a second biological response comprises emitting near infrared light to act as an anti-inflammatory.

20: The method of claim 14, wherein activating a first biological response comprises emitting ultraviolet light to act as a germicide, and wherein activating a second biological response comprises emitting near infrared light to promote cellular proliferation.

21: The method of claim 14, wherein activating a first biological response comprises emitting ultraviolet light to act as a germicide, and wherein activating a second biological response comprises emitting near infrared light to reduce pain.

22: The method of claim 14, wherein activating a first biological response comprises photactivating a pharmaceutical agent, and wherein activating a second biological response comprises heating a local area of the target structure.

23: The method of claim 1, wherein providing within the subject one or more light emitters comprises administering to said subject as the one or more light emitters at least one energy modulation agent which adsorbs, intensifies or modifies said initiation energy.

24: The method of claim 23, wherein said energy modulation agent comprises at least one of a biocompatible fluorescing metal nanoparticle, fluorescing metal oxide nanoparticle, fluorescing metal coated metal oxide nanoparticle, fluorescing dye molecule, gold nanoparticle, silver nanoparticle, gold-coated silver nanoparticle, a water soluble quantum dot encapsulated by polyamidoamine dendrimers, a luciferase, a biocompatible phosphorescent molecule, a combined electromagnetic energy harvester molecule, and a lanthanide chelate exhibiting intense luminescence.

25: The method of claim 23, wherein said energy modulation agent comprises a down-converting agent.

26: The method of claim 25, wherein said energy modulation agent comprises inorganic materials selected from the group consisting of: metal oxides; metal sulfides; doped metal oxides; and mixed metal chalcogenides.

27: The method of claim 25, wherein said energy modulation agent comprises at least one of Y₂O₃, Y₂O₂S, NaYF₄, NaYbF₄, YAG, YAP, Nd₂O₃, LaF₃, LaCl₃, La₂O₃, TiO₂, LuPO₄, YVO₄, YbF₃, YF₃, Na-doped YbF₃, ZnS; ZnSe; MgS; CaS, CaWO₄, CaSiO₂:Pb, and alkali lead silicate including compositions of SiO₂, B₂O₃, Na₂O, K₂O, PbO, MgO, or Ag, and combinations or alloys or layers thereof.

28: The method of claim 25, wherein said energy modulation agent comprises at least one of ZnSeS:Cu, Ag, Ce, Tb; CaS: Ce,Sm; La₂O₂S:Tb; Y₂O₂S:Tb; Gd₂O₂S:Pr, Ce, F; LaPO₄.

29: The method of claim 25, wherein said energy modulation agent comprises at least one of ZnS:Ag, ZnS:Cu, Pb, and alloys of the ZnSeS.

30: The method of claim 25, wherein said energy modulation agent comprises at least one of sodium yttrium fluoride (NaYF₄), lanthanum fluoride (LaF₃), lanthanum oxysulfide (La₂O₂S), yttrium oxysulfide (Y₂O₂S), yttrium fluoride (YF₃), yttrium gallate, yttrium aluminum garnet (YAG), gadolinium fluoride (GdF₃), barium yttrium fluoride (BaYF₅, BaY₂F₈), gadolinium oxysulfide (Gd₂O₂S), calcium tungstate (CaWO₄), yttrium oxide:terbium (Yt₂O₃Tb), gadolinium oxysulphide:europium (Gd₂O₂S:Eu), lanthanum oxysulphide:europium (La₂O₂S:Eu), and gadolinium oxysulphide:promethium, cerium, fluorine (Gd₂O₂S:Pr,Ce,F), YPO₄:Nd, LaPO₄:Pr, (Ca,Mg)SO₄:Pb, YBO₃:Pr, Y₂SiO₅:Pr, Y₂Si₂O₇:Pr, SrLi₂SiO₄:Pr,Na, and CaLi₂SiO₄:Pr.

31: The method of claim 25, wherein said energy modulation agent comprises at least one of KSrPO₄:Eu²⁺, Pr³⁺, NaGdF₄:Eu, Zn₂SiO₄:Tb³⁺,Yb³⁺, β-NaGdF₄ co-doped with Ce³⁺ and Tb³⁺ ions, and Gd₂O₂S:Tm or BaYF₅:Eu³⁺.

32: The method of claim 23, wherein said energy modulation agent comprises an up converting agent.

33: The method of claim 32, wherein said energy modulation agent at least one of Y₂O₃, Y₂O₂S, NaYF₄, NaYbF₄, YAG, YAP, Nd₂O₃, LaF₃, LaCl₃, La₂O₃, TiO₂, LuPO₄, YVO₄, YbF₃, YF₃, Na-doped YbF₃, or SiO₂ or alloys or layers thereof.

34: The method of claim 1, further comprising producing a change which results in destruction, lysis or inactivation of the virus.

35: The method of claim 1, further comprising producing a change which results in wound healing.

36: The method of claim 35, further comprising producing a change which results in enhancement of tissue growth, nerve regeneration or sensory regeneration/restoration.

37: The method of claim 35, further comprising producing a change which results in a treatment of a prion, viral, bacterial, fungal, or parasitic infection.

38: The method of claim 1, wherein the initiation energy is UV radiation, visible light, IR radiation, x-rays, gamma rays, an electron beam, microwaves or radio waves.

39: The method of claim 1, wherein the at least one wavelength of the light is provided internally within the subject.

40: The method of claim 39, wherein the at least one wavelength of the light provided internally within the subject is generated from a distal end of a catheter inserted into the subject.

41: The method of claim 40, wherein the at least one wavelength of the light provided internally within the subject is generated from energy modulation agents disposed at the distal end of the catheter.

42: The method of claim 41, wherein the at least one wavelength of the light provided internally within the subject is generated from down converting materials disposed at the distal end of the catheter.

43: The method of claim 42, wherein the at least one wavelength of the light provided internally within the subject is generated from up converting materials disposed at the distal end of the catheter.

44: The method of claim 1, further comprising providing a plasmonics-active agent which enhances or modifies the initiation energy or said at least one wavelength of light.

45: The method of claim 44, wherein the plasmonics-active agent comprises metal nanostructures.

46: The method of claim 45, wherein the metal nanostructures are nanospheres, nanorods, nanocubes, nanopyramids, nanoshells, multi-layer nanoshells and combinations thereof.

47: The method of claim 1, wherein the initiation energy comprises at least one or more of x-rays, gamma rays, an electron beam, UV radiation, visible light, or infrared radiation.

48: The method of claim 1, further comprising treating with said at least one wavelength of light at least one condition selected from the group consisting of cancer, bacterial infection, parasitic infection, prion infection, fungal infection, immune rejection response, autoimmune disorder, and aplastic condition.

49: The method of claim 1, further comprising treating with said at least one wavelength of light a condition, a disorder, or a disease selected from the group consisting of cardiac ablasion, photoangioplastic condition, intimal hyperplasia, arteriovenous fistula, macular degeneration, psoriasis, acne, hopecia areata, portwine spots, hair removal, autoimmune diseases, rheumatoid and inflammatory arthritis, behavioral and cognitive disorder/condition, joint condition, Parkinson's disease, retinal injury and other ocular diseases, enlarged prostate, varicose veins, reduction or removal of fat deposits (liposuction), nerve regeneration, sensory regeneration/restoration, wound healing, chronic pain, conditions occurring in bone tissue, conditions occurring in a soft tissue and/or cartilage, and lymph node condition.

50: The method of claim 1, wherein the at least one photoactivatable drug comprise at least one pharmaceutical agent selected from the group consisting of a psoralen, pyrene cholesteryloleate, acridine, porphyrin, fluorescein, rhodamine, 16-diazorcortisone, ethidium, transition metal complexes of bleomycin, transition metal complexes of deglycobleomycin organoplatinum complexes, alloxazines, vitamin Ks, vitamin L, vitamin metabolite, vitamin precursor, naphthoquinone, naphthalene, naphthol and derivatives thereof having planar molecular conformations, porphorinporphyrin, dye and phenothiazine derivative, coumarin, quinolone, quinone, and anthroquinone.

51: The method of claim 1, wherein the at least one photoactivatable drug comprises one or more of a psoralen, a coumarin, a porphyrin, and iodonophthylazide, or a derivative thereof.

52: The method of claim 1, wherein the at least one photoactivatable drug comprises at least one pharmaceutical agent selected from the group consisting of 7,8-dimethyl-10-ribityl, isoalloxazine, 7,8,10-trimethylisoalloxazine, 7,8-dimethylalloxazine, isoalloxazine-adenine dinucleotide, alloxazine mononucleotide, aluminum (III) phthalocyanine tetrasulonate, hematophorphyrin, and phthadocyanine.

53: The method of claim 1, wherein the one or more light emitters comprise at least one of a diamond light emitter and a diamond-like carbon light emitter.

54: The method of claim 1, wherein the one or more light emitters comprise one or more coated energy modulation agents.

55: The method of claim 1, wherein the one or more coated energy modulation agents have a biocompatible coating.

56: The method of claim 55, wherein the biocompatible coating comprises poly(esters) based on polylactide (PLA), polyglycolide (PGA), polycarpolactone (PCL), poly(hydroxyalkanoate) of the PHB-PHV class, poly(ester), natural polymer, modified poly(saccharide), starch, cellulose, chitosan, polyethylene oxide, poly(ether)(ester) block copolymers, ethylene vinyl acetate copolymer or a combination thereof.

57: The method of claim 1, wherein the biocompatible coating comprises at least one of a silica, a silicate, nano-diamond film, a diamond like carbon coating, and a graphene material.

58: The method of claim 1, wherein the at least one photoactivatable drug comprises an alkylating agent and psoralen.

59: The method of claim 1, wherein the at least one photoactivatable drug comprises at least one photoactivatable hydrophobic compound of the following formula I:
X—Ar—Y I,
wherein Ar is a hydrophobic moiety, and X and Y are each independently hydrogen or a reactive group, provided that at least one of X or Y is a reactive group.

60: A method for treating a subject carrying a virus or a bacterium, the method comprising:
providing within the subject at least one photoactivatable drug for treatment of the virus or the bacterium;
applying initiation energy from at least one source to a target inside the subject;
activating directly or indirectly the at least one photoactivatable drug at the target inside the subject; and
inside the subject, reacting the activated photoactivatable drug with the virus or bacterium to inactivate the virus or the bacterium, thereby treating the subject.

61: A method for treating a subject carrying a virus or a bacterium, the method comprising:
providing within a respiratory track of the subject at least one photoactivatable drug for treatment of the virus or the bacterium;
applying initiation energy from at least one source to the respiratory track;
activating directly or indirectly the at least one photoactivatable drug at the target inside the respiratory track; and
inside the respiratory track, reacting the activated photoactivatable drug with the virus or bacterium to inactivate the virus or the bacterium, thereby treating the subject.

62: A method for treating a subject carrying a virus or a bacterium, the method comprising:
providing within lymph nodes of the subject at least one photoactivatable drug for treatment the subject carrying the virus or the bacterium;
applying initiation energy from at least one source to the lymph nodes;
activating directly or indirectly the at least one photoactivatable drug at the target inside the lymph nodes; and
inside the lymph nodes, reacting the activated photoactivatable drug with the virus or bacterium to inactivate the virus or the bacterium, thereby treating the subject.
